(19) United States
(12) Patent Application Publication
(10) Pub. No.: US 2010/0210590 A1
(43) Pub. Date: Aug. 19, 2010

(54) COMPOSITIONS AND TREATMENTS FOR SEIZURE-RELATED DISORDERS

(75) Inventors: Martin D. Watterson, Chicago, IL (US); Linda J. Van Eldik, Chicago, IL (US); Mark S. Wainwright, Evanston, IL (US)

Correspondence Address: MILLEN, WHITE, ZELANO & BRANIGAN, P.C., 2200 CLARENDON BLVD., SUITE 1400, ARLINGTON, VA, 22201, US

(73) Assignee: NORTHWESTERN UNIVERSITY, EVANSTON, IL (US)

(21) Appl. No.: 12/529,357

(22) PCT Filed: Feb. 29, 2008
(86) PCT No.: PCT/US08/55495

§ 371 Date: Jan. 27, 2010

Related U.S. Application Data
(60) Provisional application No. 60/904,606, filed on Mar. 2, 2007.
(60) Provisional application No. 60/004,385, filed on Sep. 27, 1995.

(51) Int. Cl. A61K 31/695; A61K 31/50; A61K 31/502; A61K 31/501; A61K 31/5377; A61K 31/551; A61K 31/683; A61P 25/08

(52) U.S. Cl. 514/63; 514/247; 514/248; 514/252.03; 514/236.5; 514/252.04; 514/252.02; 514/252.05; 514/218; 514/85

(57) ABSTRACT

The present invention relates to therapeutic and/or prophylactic uses of pyridazine compounds and to pharmaceutical compositions containing one or more of these compounds as an active component for treating a disorder characterized by conduction disturbances, electroconvulsions and/or seizures, in particular epilepsy, more particularly pediatric epilepsy. In an aspect of the invention, the pyridnsine compound has the Formula (Ia) or (Ib) wherein R¹, R², R³, R⁴, R⁵, R⁶ and R⁷ are as defined in the description.

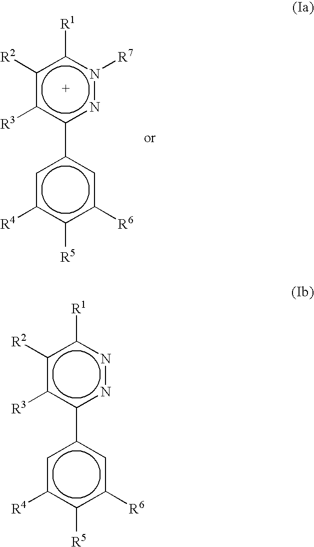

This invention was made with government support under Grant No. AG028561 and Grant No. NS044998 awarded by the “National Institute of Health.” The U.S. government has certain rights in the invention.

### FIELD OF INVENTION

The invention relates to compositions and methods for treatment of patients with seizure-related disorders, especially epilepsy.

### BACKGROUND OF INVENTION

Epilepsy is a common but devastating disorder that affects millions of people worldwide. Epilepsy is a chronic neurological condition characterized by transient but recurring excessive or abnormal disruptions to neural activity. These disruptions can be manifested as motor, convulsion, sensory or psychic symptoms, particularly in the form of seizures and loss of consciousness.

Epilepsy syndromes have been classified into more than 40 distinct types based upon characteristic symptoms, types of seizure, cause, age of onset and EEG patterns. These include, but are not limited to, absence epilepsy, psychomotor epilepsy, temporal lobe epilepsy, frontal lobe epilepsy, occipital lobe epilepsy, parietal lobe epilepsy, Lennox-Gastaut syndrome, Rasmussen's encephalitis, childhood absence epilepsy, Ramsay Hunt Syndrome type II, benign epilepsy syndrome, benign infantile encephalopathy, benign neonatal convulsions, early myoclonic encephalopathy, progressive epilepsy and infantile epilepsy.

Seizure sufferers are frequently limited in the kinds of activities they may participate in. Seizures can prevent people from driving, working or otherwise participating in much of what society has to offer. Some sufferers have serious seizures so frequently that they are effectively incapacitated. Furthermore, these are often progressive and can be associated with degenerative disorders and conditions. Over time, seizures often become more frequent and more serious, and in particularly severe cases, are likely to lead to deterioration of other brain functions as well as physical impairments.

Although a number of anti-convulsive therapies have been developed for the control of epilepsy and other disorders involving seizures, seizures remain uncontrolled in approximately one-third of patients with epilepsy, for example, and treatment failures are common (Loscher, W. and Schmidt, D., 2002, Epilepsy Res. 50:3-16). Of even greater importance is that patients often become refractory to a drug over time. In addition, many anti-seizure agents cause unwanted side effects, neurotoxicities, and drug interactions. Accordingly, a continuing need exists for pharmaceutical compositions that treat or prevent conditions involving seizures such as epilepsy, and its associated symptoms with minimal side effects.

### SUMMARY OF INVENTION

The present invention relates to therapeutic and/or prophylactic uses of pyridazine compounds and to pharmaceutical compositions containing one or more of these compounds as an active component for treating Seizure-Related Disorders, including Epilepsy.

The invention provides a composition comprising a pyridazine compound in a therapeutically effective amount for treating a Seizure-Related Disorder in a subject. In particular, the invention provides a composition comprising a pyridazine compound in a therapeutically effective amount for treating Epilepsy in a subject. The compositions of the invention generally comprise a pyridazine compound in a pharmaceutically acceptable carrier, excipient, or vehicle.

In aspects, a pharmaceutical composition of the invention comprises a therapeutically effective amount of a pyridazine compound to provide a beneficial effect, in particular a sustained beneficial effect following treatment.

In other aspects, a pharmaceutical composition comprises a pyridazine compound with a favorable pharmacological profile which makes the compounds particularly suitable in patients with enhanced need of safety and tolerability such as pediatric patients and/or patients subject to long term treatment.

The invention further provides methods for preparing a composition of the invention. In an aspect, the invention provides a method of preparing a pharmaceutical composition comprising a pyridazine compound adapted for use in a disorder disclosed herein. A method can comprise mixing one or more pyridazine compound and optionally a pharmaceutically acceptable carrier, excipient, or vehicle. A pharmaceutically acceptable carrier, excipient, or vehicle may be selected that is effective to physically stabilize the pyridazine compound(s). After compositions have been prepared, they can be placed in an appropriate container and labeled for treatment of an indicated condition. For administration of a composition of the invention, such labeling would include amount, frequency, and method of administration.

In some aspects, the invention provides methods to make commercially available pills, tablets, caplets, soft and hard gelatin capsules, lozenges, sachets, cachets, vegicaps, liquid drops, elixirs, suspensions, emulsions, solutions, syrups, aerosols (as a solid or in a liquid medium) suppositories, sterile injectable solutions, and/or sterile packaged powders, which contain a pyridazine compound adapted for use in a disorder disclosed herein.

The invention also contemplates the use of one or more pyridazine compound or method of the invention to prevent and/or ameliorate disorder severity, disorder symptoms, and/or reduce periodicity of recurrence of a disorder disclosed herein.

Therefore, the invention contemplates the prevention and treatment, in a subject, of a disorder disclosed herein, using a pyridazine compound or a composition of the invention. In particular, the invention provides a method for treating a disorder disclosed herein in a subject comprising administering to the subject a therapeutically effective amount of one or more pyridazine compound or a composition of the invention. A method of the invention can be used therapeutically or prophylactically in a subject susceptible to or having a predisposition to a disorder disclosed herein.

In an aspect, the invention provides a method for the prevention and/or intervention of a disorder disclosed herein in a subject comprising administration of at least one pyridazine compound or composition of the invention to the subject.

The invention provides a method of treating a disorder disclosed herein comprising administering at least one pyridazine compound or a composition of the invention to a subject in need thereof to thereby produce beneficial effects, in particular sustained beneficial effects following treatment. In an embodiment, the compound or composition is administered orally or systemically.

In an aspect, the invention provides a method for ameliorating progression of a disorder or obtaining a less severe stage of a disorder disclosed herein in a subject suffering from such disorder comprising administering a therapeutically effective amount of one or more pyridazine compound or a composition of the invention.

The invention relates to a method of delaying the progression of a disorder disclosed herein comprising administering a therapeutically effective amount of one or more pyridazine compound or a composition of the invention.

The invention also relates to a method of increasing survival of a subject suffering from a disorder disclosed herein comprising administering a therapeutically effective amount of one or more pyridazine compound or a composition of the invention.

In an embodiment, the invention relates to a method of improving the lifespan of a subject suffering from a disorder disclosed herein comprising administering a therapeutically effective amount of one or more pyridazine compound or a composition of the invention.

In particular embodiments of the invention, the compositions and methods are useful in the prevention, palliation, and/or treatment of Seizure-Related Disorders (e.g. seizures, conduction disturbances, and electroconvulsive disorders) and their manifestations irrespective of the origin of the condition in a subject.

The invention provides methods for the prevention, palliation and/or treatment of epilepsy in a pediatric patient comprising inhibiting glial activation. In a particular aspect, the invention provides methods for the prevention, palliation and/or treatment of epilepsy in a pediatric patient comprising reducing pro-inflammatory cytokines (e.g. IL-β and S100B) following early-life seizures.

A treatment method of the invention may be sustained over several days, weeks, months or years thereby having a major beneficial impact on the severity of a disorder or and its complications.

The invention also contemplates the use of one or more pyridazine compound as a medicament or for the preparation of a medicament for preventing and/or treating a disorder disclosed herein.

The invention additionally provides uses of a pharmaceutical composition of the invention as a medicament or in the preparation of medicaments for the prevention and/or treatment of a disorder disclosed herein. In aspects of the invention, the medicaments provide beneficial effects, preferably sustained beneficial effects following treatment. A medicament may be in a form suitable for consumption by a subject, for example, a pill, tablet, caplet, soft and hard gelatin capsule, lozenge, sachet, cachet, vegicap, liquid drop, elixir, suspension, emulsion, solution, syrup, aerosol (as a solid or in a liquid medium) suppository, sterile injectable solution, and/or sterile packaged powder.

A composition or method of the invention may be administered to a healthy subject or a subject suffering from a disorder disclosed herein. Accordingly, in an embodiment, a pyridazine compound or a composition of the invention is to be administered before or after the onset of symptoms in a subject.

The invention also provides a kit comprising a pyridazine compound or a pharmaceutical composition of the invention in kit form. In an aspect, the invention provides a kit comprising one or more pyridazine compound or composition of the invention, a container, and instructions for use in treating and/or preventing a disorder disclosed herein.

These and other aspects, features, and advantages of the present invention should be apparent to those skilled in the art from the following detailed description.

### DESCRIPTION OF THE FIGURES

FIG. 1. (A) Latency to seizure onset; (B), survival and; (C, D), quantification of neuronal injury following kainic acid (KA)-induced seizures in the immature and adult rat. (A) Latency of seizure onset (minutes±SEM) on postnatal day (P)15 (KA-PBS) is reduced compared to adult (P45) seizures (PBS-KA). Early-life seizures increase susceptibility to seizures in adulthood shown by reduced latency of seizures in the ‘two hit’ (P15 and P45) seizure group (KA-KA) compared to the adult seizure (PBS-KA) group. (B) Kaplan-Meier survival curves over 40-day recovery following administration of KA or vehicle (PBS) on P15 are significantly different (log Rank Test, p<0.05). Survival is significantly decreased in the KA-KA group following the second hit of KA. (C) Representative photomicrograph demonstrating Fluoro-Jade B (FJB) fluorescent dead and dying neurons (green) counterstained with DAPI in the CA1 field of the hippocampus of a ‘two-hit’ (KA-KA) animal. (D) Quantification of FJB-fluorescence after 40-day recovery in the hippocampus of control (PBS-PBS) and KA-PBS groups shows no evidence of injury. ‘Two-hit’ (KA-KA) animals show greater injury compared to adult animals first exposed to KA (PBS-KA). (E) Regions of interest for cell counts in axial sections of the hippocampus. Digital images of CA1, CA2, CA3, dentate gyrus (DG), and polymorph dentate gyrus (PoDG) were obtained at low power (10×). Each image was analyzed for positive staining, and results added to represent total hippocampal staining. ***p<0.001; ** p<0.01 vs PBSPBS by ANOVA. Scale bars, (C) 100 μm; (E) 200 μm.

FIG. 2. KA-induced seizures increase hippocampal proinflammatory cytokine protein levels after 24-hour recovery. (A) Significant increases in IL-1β were present in both newborns (P15 KA) and adults (P45 KA). No intergroup differences were found for IL-6 or TNF-α (B, C). (D) S100B levels were significantly increased compared to controls following seizures both in newborns and adults. P values determined by ANOVA.

FIG. 3. (A) KA-induced seizures in newborns result in impairment in hippocampal-linked-task. Serial measurement of spontaneous alternation of rats in Y-maze after early-life seizures (KA-PBS) shows reduction compared to controls (PBS-PBS) over 40-day recovery. (B-F) KA-induced seizures on P15 result in prolonged increase in hippocampal expression of GFAP on P55. Representative photomicrographs illustrating GFAP immunostaining in the CA1 field of the hippocampus of controls (B, PBS-PBS), early-life seizures (C, KA-PBS), adult seizures (D, PBS-KA), and ‘two-hit’ (E, KA-KA) animals. Morphology of GFAP-positive astrocytes in the CA1 of KA-PBS, PBS-KA and KA-KA groups resembles activated glia compared controls (PBS-PBS). (F) Quantification of GFAP-immunoreactive cells in combined regions of the hippocampi in the four groups. All three KA-treated groups showed a significant increase in GFAP expression compared to controls. (G-K) KA-induced seizures on P15 result in prolonged increase in hippocampal expression of S100B over 40-day recovery. (G-J) Representative photomicrographs illustrating S100B immunostaining in the CA1 field of the hippocampus of PBS-PBS (G), KA-PBS (H), PBS-KA (I), and KA-KA (J) animals. (K) Quantification of S100B-immunoreactive cells in combined regions of the hippocampi in the four groups. All three KA-treated groups showed a significant increase in S100B expression compared to controls. Counterstain is hematoxylin. Insets (B-E; G-J) represent higher magnification photomicrographs of CA1. ***p<0.001 vs PBS-PBS; **p<0.01 vs PBS-PBS by ANOVA. Scale bars, (B) 200 μm; Inset (B) 100 μm.

FIG. 4. KA-induced microglial activation in adult animals is enhanced by early-life seizures. (A-D) Representative photomicrographs illustrating Ibal immunostaining in the CA1 field of the hippocampus of PBS-PBS (A), KA-PBS (B), PBS-KA (C) and KA-KA (D) animals. Insets (A-D) are higher magnification photomicrographs of CA1. (E) Quantification of Iba1-immunoreactive cells in combined regions of the hippocampi in the four groups shows no long-term increase in microglial activation following early-life seizures (KA-PBS). The ‘two-hit’ (KA-KA) group showed significantly greater microglial activation compared to the adult seizure group (PBS-KA). Values in both groups were increased compared to controls (PBS-PBS). ***p<0.001 vs PBSPBS; **p<0.01 vs PBS-PBS by ANOVA. Scale bars (A) 200 μm; Insets (A-D) 100 μm.

FIG. 5. Hippocampal factor H (FH) and clusterin levels following KA exposure. (A) Western blot micrographs and relative quantification of FH blot density demonstrate no significant changes in FH protein levels 24-hr following KA-induced seizures in newborns and adults. (B) Representative photomicrograph of FH immunostaining in the CA1 field of the hippocampus of ‘two-hit’ (KA-KA) animals on P55. (B inset) Photomicrograph showing a FH-immunoreactive astrocyte. (C) Quantification of FH-immunoreactive cells on P55 in combined regions of the hippocampi in the long-term recovery groups shows no significant intergroup differences. (D) Western blot micrographs and relative quantification of clusterin blot density show a significant increase in hippocampal clusterin protein levels 24-hr following seizures on P15 and P45. (E-G) Representative photomicrographs demonstrating clusterin immunostaining in the CA1 field of the hippocampus of long-term recovery groups, controls (E inset, PBS-PBS), early-life seizures (E, KA-PBS), adult seizures (F, PBS-KA; Inset, higher power view of clusterin-immunoreactive cells in CA1), and ‘two-hit’ (G, KA-KA) animals. (H) Quantification of clusterin-immunoreactive cells on P55 in combined regions of the hippocampi in the long-term recovery groups shows a significant increase in the ‘two-hit’ (KA-KA) group compared to the adult seizure (PBS-KA) animals. (I, J) Representative photomicrographs demonstrating that NeuN-positive cells do not co-stain with clusterin. (I) NeuN (gray) and clusterin, (red) do not co-label. (J) Immunofluorescent detection shows a similar pattern of NeuN (green) and clusterin (red). (K) Immunofluorescent double-labeling with GFAP (green) and clusterin (red) shows that clusterin-positive cells are not astrocytes. Clusterin-positive cells are Ibal-immunoreactive (L), suggesting that these cells are microglia. (H) ***p<0.001 vs PBS-PBS by ANOVA. (B) Scale bars (B,F,G) 200 μm; (E, J) 100 μm; (I,L) 50 μm.

FIG. 6. KA-induced seizures in P45 animals result in an increase in glial-specific glutamate transporters, GLAST and GLT-1. (A-C) Representative photomicrographs illustrating GLAST immunostaining in the field CA1 of the hippocampus of PBS-PBS (A inset), KA-PBS (A), PBSKA (B) and KA-KA (C) animals. Inset (C) shows higher power view of GLAST-immunoreactive cells in CA1. (D) Quantification of GLAST-immunoreactive cells on P55 in combined regions of the hippocampi in the long-term recovery groups demonstrates increased expression in PBS-KA and KA-KA compared to controls (PBS-PBS). PBS-KA and KA-KA are not significantly different from each other. GLT-1 results were similar to GLAST (data not shown). **p<0.01 vs PBS-PBS; *p<0.05 vs PBS-PBS by ANOVA. Scale bars 200 μm; (Inset C) 100 μm.

FIG. 7. (A) Timeline of Minozac (Mzc) administration and Y-maze testing. Following intraperitoneal (ip) administration of either PBS or kainic acid (KA) on P15, animals were treated with Mzc (5 mg/kg, ip) or Saline diluent (Sal), at 3 hr and 9 hr recovery. One cohort of animals from each group (PBS-Sal, KA-Sal and KA-Mzc) was sacrificed at 12 hr for assessment of changes in hippocampal pro-inflammatory cytokines. Remaining animals were allowed to recover for 30 days (P45). In these groups, Y-maze testing for hippocampal-dependent behavior was initiated at weaning age (P21, 6 day recovery) and continued until sacrifice. (B) Serial measurement of spontaneous alternation in Y-maze after early-life seizures shows protection by Mzc over 30 day recovery. ***p<0.001 vs KA-Sal by ANOVA.

FIG. 8. Effect of Minozac (Mzc) on acute increase in proinflammatory cytokines following KA-induced seizures on P15 (A-D) and glial activation after 30 day recovery (E-L). (A) Administration of Mzc following early-life seizures suppressed the acute increase in IL-1β seen in untreated animals (KA-Sal). Similar results were seen for IL-6 (B), TNF-α (C), and S100B (D). Tissues for brain homogenates were collected at 12 hr following KA injection. (E-L) Minozac (Mzc) suppresses long-term glial activation response to KA-induced early-life seizures after 30 day recovery from KA exposure on P15. (E-G) Representative photomicrographs of GFAP immunostaining in the CA1 field of control (E, PBS-Sal), early-life seizure (F, KA-Sal) and Mzc-treated (G, KA-Mzc) animals. (H) Quantification of GFAP-positive cells in the hippocampus shows Mzc-treated animals (KA-Mzc) have significantly less glial activation compared to untreated animals (KA-Sal). (I-L) Representative photomicrographs of S100B immunostaining in the CA1 field of PBS-Sal (I), KA-Sal (J) and KA-Mzc (K) animals. (L) Quantification of S100B-immunopositve cells show that Mzc suppressed the increased S100B expression seen in KA-Sal. Data (D) expressed as % Control±SEM. Data (H, L) are % immuno-positive cells±SEM. *p<0.05 vs KA-Sal, **p<0.01 vs KA-Sal; ***p<0.001 vs KA-Sal by ANOVA. Scale bars (E) 200 μm; (Insets F,G,J,K) 100 μm.

FIG. 9. Proposed schema for the role of astrocyte and microglial activation in enhanced seizure susceptibility. Summary of effects of kainate administration on postnatal (P) day 15 (A), and P45 (B) including ‘two-hits’ (P15 and 45) (B). On P15, a transient increase in proinflammatory cytokines is followed by microglial activation and sustained (40 day) astrocyte activation. A similar pattern of cytokine and microglial activation occurs on P45 when astrocyte glutamate transporters also increase. Importantly, the ‘two-hit’ group exposed to kainate on P15 and P45 show enhanced microglial activation (B, green dashed line).

FIG. 10 depicts a production scheme for synthesis of 2-(4-(4-methyl-6-phenylpyridazin-3-yl)piperazin-1-yl)pyrimidine dihydrochloride salt (MW01-9-034WH, also referred to herein as “Minozac”). Reagents and conditions: (a) N₂H₄, EtOH, reflux, (b) CuCl₂, CH₃CN, reflux, (c) POCl₃, CH₃CN, reflux, (d) 1-(2-pyrimidyl)piperazine, water, reflux, (e) HCl, isopropanol.

### DETAILED DESCRIPTION OF EMBODIMENTS OF THE INVENTION

For convenience, certain terms employed in the specification, examples, and appended claims are collected here.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition comprising “a compound” includes a mixture of two or more compounds.

As used herein the terms “administering” and “administration” refer to a process by which a therapeutically effective amount of a compound or composition contemplated herein is delivered to a subject for prevention and/or treatment purposes. Compositions are administered in accordance with good medical practices taking into account the subject's clinical condition, the site and method of administration, dosage, patient age, sex, body weight, and other factors known to physicians.

The term “treating” refers to reversing, alleviating, or inhibiting the progress of a disorder, or one or more symptoms of such disorder, to which such term applies. Depending on the condition of the subject, the term also refers to preventing a disorder, and includes preventing the onset of a disorder, or preventing the symptoms associated with a disorder. A treatment may be either performed in an acute or chronic way. The term also refers to reducing the severity of a disorder or symptoms associated with such disorder prior to affliction with the disorder. Such prevention or reduction of the severity of a disorder prior to affliction refers to administration of a compound or composition of the present invention to a subject that is not at the time of administration afflicted with the disorder. “Preventing” also refers to preventing the recurrence of a disorder or of one or more symptoms associated with such disorder. “Treatment” and “therapeutically,” refer to the act of treating, as “treating” is defined above. The purpose of prevention and intervention is to combat the disorder and includes the administration of an active compound to prevent or delay the onset of the symptoms or complications, or alleviating the symptoms or complications, or eliminating the disorder.

In aspects of the invention where the disorder is epileptogenesis, in particular epilepsy, treatment may comprise (i) partial or complete reversal of epileptogenesis, (ii) prevention of, or decrease or slowing of the rate of epileptogenesis, (iii) inhibition or slowing of the rate of biochemical processes which take place during epileptogenesis; and/or (iv) prevention, slowing, halting and/or reversing the process of epileptogenesis.

The terms “subject”, “individual”, or “patient” are used interchangeably herein and refer to an animal preferably a warm-blooded animal such as a mammal. Mammal includes without limitation any members of the Mammalia. A mammal, as a subject or patient in the present disclosure, can be from the family of Primates, Carnivora, Proboscidea, Perissodactyla, Artiodactyla, Rodentia, and Lagomorpha. In a particular embodiment, the mammal is a human. In other embodiments, animals can be treated; the animals can be vertebrates, including both birds and mammals. In aspects of the invention, the terms include domestic animals bred for food or as pets, including equines, bovines, sheep, poultry, fish, porcines, canines, felines, and zoo animals, goats, apes (e.g. gorilla or chimpanzee), and rodents such as rats and mice.

In aspects of the invention, the terms refer to organisms to be treated by the methods of the present invention. In the context of particular aspects of the invention, the term “subject” generally refers to an individual who will receive or who has received treatment (e.g., administration of a pyridazine compound(s) or compositions) for a disorder disclosed herein.

Typical subjects for treatment include persons afflicted with or suspected of having or being pre-disposed to a disorder disclosed herein, or persons susceptible to, suffering from or that have suffered a disorder disclosed herein. A subject may or may not have a genetic predisposition for a disorder disclosed herein, such as Epilepsy. In certain aspects of the invention the subject is a pediatric patient. In certain aspects, a subject may be a healthy subject.

As utilized herein, the term “healthy subject” means a subject, in particular a mammal, having no diagnosed disorder, infirmity, or ailment disclosed herein.

As used herein, the terms “co-administration”, “combination treatment”, and “administering in combination” refer to the administration of one or more pyridazine compound and additional therapeutic agent or therapies to a subject. In aspects, the administration of two or more agents/therapies is concurrent. In other aspects, a first agent/therapy is administered prior to a second agent/therapy. In this aspect, each component may be administered separately, but sufficiently close in time to provide the desired effect, in particular a beneficial, additive, or synergistic effect. The formulations, routes of administration and the appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

A “beneficial effect” refers to an effect of a pyridazine compound or a composition of the invention, including favorable pharmacological and/or therapeutic effects, and improved biological activity. In aspects of the invention, the beneficial effects include without limitation enhanced stability, a longer half life, and/or enhanced uptake. In other aspects the beneficial effects include one or more of the following: (a) reduction or amelioration of neuronal dysfunction or injury following early-life seizures; (b) reduction or suppression of pro-inflammatory cytokines; (c) reduction in astrocyte activation; (d) reduction in GFAP immunoreactive cells; (e) reduction in microglial activation and (f) reduction in glial activation and impairment of hippocampal-dependent behavior. A beneficial effect can be a statistically significant effect in terms of statistical analysis of an effect of a pyridazine compound or a composition of the invention, versus the effects without the compound or composition that is not within the scope of the invention. Statistically significant” or “significantly different” effects or levels may represent levels that are higher or lower than a standard. In aspects of the invention, the difference may be 1.5, 2, 3, 4, 5, 6, 7, 8, 9, 10, 15, 20, 25 or 50 times higher or lower compared with the effect obtained without a pyridazine compound or a composition of the invention.

In aspects, the beneficial effect is a “sustained beneficial effect” where the beneficial effect is sustained for a prolonged period of time after termination of treatment. A treatment can be sustained over several days, weeks, months or years thereby having a major beneficial impact on the severity of the disorder and its complications. In aspects of the invention, a beneficial effect may be sustained for a prolonged period of at least about 1 to 3 days, 2 to 4 weeks, 2 to 5 weeks, 3 to 5 weeks, 2 to 6 weeks, 2 to 8 weeks, 2 to 10 weeks, 2 to 12 weeks, 2 to 14 weeks, 2 to 16 weeks, 2 to 20 weeks, 2 to 24 weeks, 2 weeks to 12 months, 2 weeks to 18 months, 2 weeks to 24 months, or several years following treatment. The period of time a beneficial effect is sustained may correlate with the duration and timing of the treatment. A subject may be treated continuously for about or at least about 1 to 3 days, 1 week, 2 to 4 weeks, 2 to 6 weeks, 2 to 8 weeks, 2 to 10 weeks, 2 to 12 weeks, 2 to 14 weeks, 2 to 16 weeks, 2 weeks to 6 months, 2 weeks to 12 months, 2 weeks to 18 months, or several years, periodically or continuously.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers etc. include but are not limited to saline, buffered saline, dextrose, water, glycerol, ethanol, and combinations thereof. The use of such media and agents for an active substance is well known in the art.

“Therapeutically effective amount” relates to the amount or dose of an active pyridazine compound or composition of the invention that will lead to one or more desired effects, in particular, one or more beneficial effects, more particularly therapeutic effects. A therapeutically effective amount of a substance can vary according to factors such as the disorder state, age, sex, and weight of the subject, and the ability of the substance to elicit a desired response in the subject. A dosage regimen may be adjusted to provide the optimum therapeutic response (e.g. sustained beneficial effects). For example, several divided doses may be administered daily or the dose may be proportionally reduced as indicated by the exigencies of the therapeutic situation. Generally, when treating a CNS disorder, an effective amount of compound or composition is that amount sufficient to pass across the blood-brain barrier of the subject to interact with relevant receptor sites in the brain of the subject.

A “pyridazine compound” refers to a compound of the formula I, II, III, IV, or V, or a compound depicted in Table 1, 2, 3, 4, or 5, in particular Table 2, 3, 4, or 5. In aspects of the invention a pyridazine compound refers to a pyridazinyl radical pendant with an aryl or substituted aryl, a heteroaryl or substituted heteroaryl. In some aspects the term includes the structures disclosed in US Patent Application Serial Numbers 20030176437 and 20060073472.

In aspects, a pyridazine compound that demonstrates beneficial effects, in particular statistically significant beneficial effects is selected for use in the present invention.

In aspects of the invention, a compound of the following formula Ia or Ib is employed.

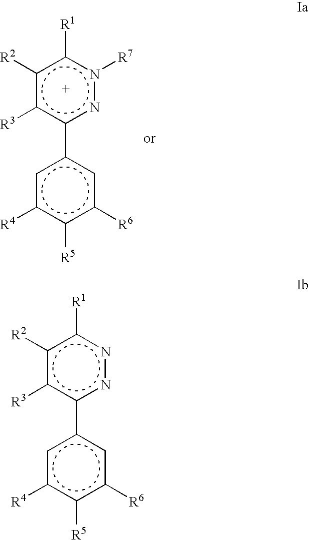

wherein R¹, R², and R³ are independently substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide; R⁷ is substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide or R⁷ may be absent and there is a double bond between N at position 1 and C at position 6; R⁴, R⁵, and R⁶ are independently hydrogen, alkyl, alkoxy, halo, or nitro; or R¹ and R², R¹ and R⁷, or R² and R³ may form a heteroaryl or heterocyclic ring; or an isomer or a pharmaceutically acceptable salt thereof.

In an aspect, a compound of the Formula Ia or Ib is employed wherein: (a) R¹ is optionally substituted halo, hydroxyl, alkyl, alkenyl, alkoxy, cyano, amino, cycloalkyl, sulfonyl, sulfinyl, sulfenyl, thioaryl, thioalkyl, carbonyl, silyl, piperazinyl, piperidinyl, pyrrolidinyl, morpholinyl, —SR²⁰ wherein R²⁰ is optionally substituted alkyl, carbonyl, carboxyl, carbamoyl, aryl, heterocylic, or heteroaryl; (b) R² is optionally substituted halo, hydroxyl, alkyl, alkenyl, alkoxy, carbonyl, carboxyl, phenyl, benzyl, amino, aryl, cyano, —COH, piperazinyl, alcohol, piperidinyl, morpholinyl, or naphthyl;(c) R³ is optionally substituted hydrogen, halo, hydroxyl, alkyl, alkenyl, alkoxy, phenyl, piperazinyl, piperidinyl, pyrrolidinyl, morpholinyl, thiol, sulfenyl, sulfonyl, sulfinyl, or nitro; (d) R⁴ is hydrogen, halo, or nitro; (e) R⁵ is optionally substituted hydrogen, halo, alkoxy, or amido;(f) R⁷ is substituted or unsubstituted hydrogen halo, hydroxyl, alkyl, alkenyl, alkoxy, carboxy, morpholino, imidazolyl, piperazinyl, piperidinyl, pyrrolidinyl, morpholinyl or R⁷ is absent and there is a double bond between N at position 1 and C at position 6; and/or (g) R¹ and R², R¹ and R⁷ or R² and R³ may form a substituted or unsubstituted heteroaryl or heterocyclic ring.

In another aspect of the invention a compound of the Formula Ia or Ib is employed wherein R¹ is C¹ or Br, —NH₂, alkyl, —CN, ═S, silyl, sulfonyl, thioalkyl, thioaryl, piperazinyl, piperidinyl, morpholinyl, pyrrolyl, or pyrrolidinyl, which may be optionally substituted with halo, ═O, alkoxy, alkenyl, alkyl, substituted alkyl, —CN, —SR²¹ wherein R²¹ is optionally substituted methyl, ethyl, phenyl, heterocylic, or heteroaryl, or —CO substituted with phenyl or substituted phenyl.

In another aspect of the invention a compound of the Formula Ia or Ib is employed wherein R² is carbonyl, piperazinyl, morpholinyl, sulfonyl, sulfinyl, sulfenyl, or phenyl, —CN, —COH, —CH₂OH, —OCH₂CH₃, or alkyl which may be optionally substituted with alkyl, alkoxy, amino, halo, phenyl, substituted phenyl, benzyl, hydroxyl, amino, piperidinyl, or morpholinyl.

In another aspect of the invention a compound of the Formula Ia or Ib is employed wherein R³ is piperazinyl; substituted piperzinyl; alkyl which may optionally be substituted with amino; phenyl; substituted phenyl; amino which may be optionally substituted with alkyl or alkylamine (e.g., NHCOOC(CH₃)₃), carboxyl, or substituted carboxyl; hydroxyl; or nitro.

In another aspect of the invention a compound of the Formula Ia or Ib is employed wherein R⁴ is nitro or hydrogen.

In another aspect of the invention a compound of the Formula Ia or Ib is employed wherein R⁵ is hydrogen, halo, —OCH₂CH₂CH₂NHCOOC(CH₃)₃, or —OCH₃.

In another aspect of the invention a compound of the Formula Ia or Ib is employed wherein R⁷ is alkyl, morpholinyl, benzyl, imidazolyl, —CH₂COOCH₂CH₃, CH₂C═COOCH₂CH₃, CH₂CH₂CH₂SO₂OH, CH₂CH₂CH₂SO₃—, CH₂CH₂CH₂CH₂PO(OH)₂, or CH₂CH₂CH₂PO(OH)₂.

In another aspect of the invention a compound of the Formula Ia or Ib is employed wherein R⁷ is absent and there is a double bond between N at position 1 and C at position 6.

In a further aspect, a compound of the Formula Ia is employed wherein R¹, R², R³, and R⁷ are independently substituted aliphatic, lower alkyl substituted amino, lower alkyl substituted halogen, cycloaliphatic, or substituted cycloaliphatic.

In a still further aspect of the invention a compound of the Formula Ia or Ib is employed wherein R¹ is a piperazinyl which may be substituted (e.g., with a pyrimidinyl moiety); halo; amino which may be substituted; cyano; —SR²² wherein R²² is alkyl or aryl (e.g. phenyl) which may be substituted (e.g., halo); substituted alkyl [e.g., alkyl substituted with halogen, such as CH(Br)₂]; morpholinyl; pyrrolyl which may be substituted; hydroxyl; —OR²⁸ wherein R²⁸ is alkyl; —C═CHR³⁰ wherein R³⁰ is alkyl; or pyrrolidinyl.

In a still further aspect of the invention a compound of the Formula Ia or Ib is employed wherein R² is hydrogen; morpholinyl; piperazinyl which may be substituted (e.g., with a pyrimidinyl moiety); phenyl; alkyl; alkoxy (e.g. CH(OCH₃)₂); substituted alkyl; substituted aryl (e.g., phenyl); cyano; or hydroxyl.

In another aspect of the invention a compound of the Formula Ib is employed wherein R¹ is pyridinyl, and R² is an N-substituted piperzinyl.

In another embodiment a compound of the Formula Ib is employed wherein R¹ is amino substituted with alkyl or cycloalkyl and R² is pyridinyl.

In a still further aspect of the invention a compound of the Formula Ia or Ib is employed wherein R³ is hydrogen; hydroxyl; alkyl which may be substituted (e.g., halo); amino which may be substituted; —COR³¹ wherein R³¹ is hydrogen, hydroxyl, alkoxy (e.g. —OCH₃); or, aryl (e.g. phenyl) which may be substituted (e.g., alkyl).

In a still further aspect of the invention a compound of the Formula Ia or Ib is employed wherein R⁴ is hydrogen or halo; R⁵ is hydrogen or halo; R⁶ is hydrogen or halo.

In a still further aspect of the invention a compound of the Formula Ia is employed wherein R⁷ is hydrogen; alkyl which may be substituted (e.g. with phenyl); —CH₂CH₂COOR³² wherein R³² is alkyl, —CH₂C═COOR³³ wherein R³³ is alkyl, CH₂CH₂CH₂S(O)₂OH, morpholinyl, benzyl, imidazolyl, or [CH₂]nPO(OH)₂ wherein n is 1 to 6, in particular 3 or 4.

In a still further aspect of the invention a compound of the Formula Ia or Ib is employed wherein R¹ and R² form a piperidinyl ring which may optionally be substituted with a carboxyl.

In a still further aspect of the invention a compound of the Formula Ia is employed wherein R¹ and R⁷ form a pyrimidinyl ring which may optionally be substituted with alkyl, aryl, halo, or hydroxyl.

In a particular aspect, a compound of the formula Ia or Ib is employed wherein R¹ is —NR³⁴R³⁵ wherein R³⁴ is hydrogen or alkyl, and R³⁵ is hydrogen, alkyl, carbonyl, aryl, amino, cycloalkane, heterocylic, or heteroaryl which may be substituted. In embodiments R³⁵ may comprise or be selected from the group consisting of hydrogen, C₁-C₆ alkyl (e.g. methyl or ethyl) which may be substituted with optionally substituted hydroxyl, alkyl, amino, carbonyl, carboxyl, morpholinyl, isoquinolinyl, or an amino which may be substituted with one or more of optionally substituted alkyl, benzyl, carboxyl, alcohol group, heteroaryl or heterocyclic, a propanol group, phenyl which may be optionally substituted with halo, benzyl which may be substituted with alkoxy, cyclohexyl, piperidinyl which may be substituted with optionally substituted phenyl, pyrrolidinyl or pyrrolidinylalkyl which may be substituted with alkyl, —COOR^{S} wherein R⁸ is alkyl which may be substituted, or [CH₂]ₘ-piperidinyl wherein m is 1 to 4, in particular 1 to 3 and the piperidinyl is optionally substituted with optionally substituted alkyl, phenyl, or benzyl.

In embodiments, R³⁵ is —R⁴⁴R⁴⁵ wherein R⁴⁴ is —NH[CH₂]_{w}NH wherein w is 1 to 4, in particular 2 or 3, and R⁴⁵ is piperazinyl substituted with pyrimidinyl which may be substituted, in particular substituted with alkyl.

In embodiments, R³⁵ is —R⁴⁶R⁴⁷ wherein R⁴⁶ is —[CH₂]_{w}N(CH₃) wherein w is 1 to 4, in particular 2 or 3, and R⁴⁷ is piperazinyl substituted with pyrimidinyl which may be substituted, in particular substituted with alkyl.

In an aspect of the invention, a compound of the Formula Ia or Ib is employed wherein R¹ is halo, especially chloro or bromo, R² is alkyl which may be substituted, in particular substituted with alkoxy (e.g., methoxy, dimethoxy), substituted aryl which may be substituted with alkyl, alkoxy, (e.g., benzyl, methoxy phenyl), halo (e.g. bromo or chloro), or carbonyl, a substituted or unsubstituted saturated 3 to 6-membered heteromonocylic group containing 1 to 4 nitrogen atoms [e.g., piperidinyl, and piperazinyl] or a saturated 3 to 6-membered heteromonocyclic group containing 1 to 2 oxygen atoms and 1 to 3 nitrogen atoms [e.g. morpholinyl; sydnonyl], in particular a substituted morpholinyl, piperazinyl,or piperazinyl substituted with a heteroaryl in particular an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, or pyridazinyl, especially pyrimidinyl, and optionally R³, R⁴, R⁵, R⁶, and R⁷ are hydrogen.

In another aspect of the invention, a compound of the Formula Ia is employed wherein R¹ is halo especially chloro or bromo, and R³ is a substituted or unsubstituted saturated 3 to 6-membered heteromonocylic group containing 1 to 4 nitrogen atoms [e.g., piperidinyl, and piperazinyl] or a saturated 3 to 6-membered heteromonocyclic group containing 1 to 2 oxygen atoms and 1 to 3 nitrogen atoms [e.g. morpholinyl; sydnonyl], in particular a substituted morpholinyl, piperazinyl,or piperazinyl substituted with alkyl or a heteroaryl in particular an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, or pyridazinyl, especially pyrimidinyl, or R² is a substituted amino, in particular amino substituted with alkyl or substituted alkyl, in particular alkyl substituted with alkoxy carbonyl, and optionally R², R⁴, R⁵, R⁶, and R⁷ are hydrogen.

In further aspect R¹ is halo, especially bromo or chloro, and R² and R³ form an unsaturated ring, in particular phenyl, R⁵ is a heteroaryl, in particular a substituted or unsubstituted unsaturated 5 to 6 membered heteromonocyclyl group containing 1 to 4 nitrogen atoms, in particular, pyrrolyl, pyrrolinyl, imidazolyl, pyrazolyl, especially imidazolyl, and optionally R⁴, R⁶ and R⁷ are hydrogen.

In a further aspect, R¹ is halo, especially bromo or chloro, and R⁴ is nitro, and optionally R², R³, R⁵, R⁶, and R⁷ are hydrogen.

In a further aspect, the invention employs a compound of the Formula Ia wherein R¹ is a thiol substituted with alkyl(thioalkyl); substituted alkyl, in particular alkyl substituted with a substituted or unsubstituted saturated 3 to 6-membered heteromonocylic group containing 1 to 4 nitrogen atoms [e.g. pyrrolidinyl, imidazolidinyl, piperidinyl, and piperazinyl] or a saturated 3 to 6-membered heteromonocyclic group containing 1 to 2 oxygen atoms and 1 to 3 nitrogen atoms [e.g. morpholinyl; sydnonyl], especially a substituted morpholinyl or piperidinyl; aryl; substituted aryl; carboxyl which may be substituted with substituted or unsubstituted aryl; optionally R² is alkyl, in particular lower alkyl; optionally R³ is alkyl, in particular lower alkyl or nitro; optionally R⁵ is alkoxy; optionally R⁷ is alkyl; and optionally R⁴, R⁵, and R⁶, are hydrogen.

In a further aspect of the invention, a compound of the Formula Ia is employed wherein R¹ is ═S, and optionally R² is alkyl, in particular lower alkyl, R⁵ is alkoxy, and R³,R⁴, R⁶ and R⁷ are hydrogen.

In a further aspect of the invention, a compound of the Formula Ia is employed wherein R¹ is sulfonyl which may be substituted with substituted or unsubstituted aryl, in particular substituted phenyl, and optionally R² is alkyl and R³, R⁴, R⁵, R⁶, and R⁷ are hydrogen.

In a further aspect of the invention, a compound of the Formula Ia is employed wherein R¹ is substituted or unsubstituted alkyl or alkynyl, in particular alkyl substituted with aryl, substituted aryl, halo, cyano, or alkynyl substituted with alkyl; and optionally R² is alkyl, R⁷ is alkyl, and R³, R⁴, R⁵, and R⁶ are hydrogen.

In a further aspect of the invention, a compound of the Formula Ia is employed wherein R¹ is cyano and R² is aryl or alkyl, and optionally R³, R⁴, R⁵, R⁶, and R⁷ are hydrogen.

In a further aspect of the invention, a compound of the Formula Ia is employed wherein one or both of R¹ and R² are a saturated 3 to 6-membered heteromonocyclic group containing 1 to 2 oxygen atoms and 1 to 3 nitrogen atoms [e.g. morpholinyl; sydnonyl], especially a substituted morpholinyl, and optionally R³, R⁴, R⁵, R⁶, and R⁷ are hydrogen.

In a further aspect of the invention, a compound of the Formula Ia is employed wherein R¹ is a saturated 3 to 6-membered heteromonocylic group containing 1 to 4 nitrogen atoms [e.g. pyrrolidinyl], which may be substituted with substituted or unsubstituted carboxyl; R² is alkyl or halo, and optionally R³, R⁴, R⁵, R⁶, and R⁷ are hydrogen.

In a further aspect of the invention, a compound of the Formula Ia is employed wherein R¹ is hydroxyl; R² is alkyl or substituted alkyl or R³ is a saturated 3 to 6-membered heteromonocylic group containing 1 to 4 nitrogen atoms [e.g. piperidinyl, and piperazinyl] which may optionally be substituted with a heteroaryl [e.g., pyrimidinyl], and the other of R² or R³ is hydrogen, and optionally R⁴, R⁵, R⁶, and R⁷ are hydrogen.

In a further aspect of the invention, a compound of the Formula Ia is employed wherein R¹ is a saturated 3 to 6-membered heteromonocylic group containing 1 to 4 nitrogen atoms [e.g., piperidinyl and piperazinyl] which may be substituted with carboxyl or carboxyl substituted with alkyl or alkoxy or with purinyl or substituted purinyl; R² is alkyl or substituted alkyl, in particular alkylaryl, and optionally R³, R⁴, R⁵, R⁶, and R⁷ are hydrogen.

In a further aspect of the invention, a compound of the Formula Ia is employed wherein R¹ is ═O, and R² is alkyl, alkylaryl, cyano, alkoxy, or substituted alkoxy, and optionally R³, R⁴, R⁵, R⁶, and R⁷ are hydrogen.

In a further aspect of the invention, a compound of the Formula Ia is employed wherein R¹ is alkoxy, R² is alkyl, substituted alkyl, or alkoxy, and optionally R³, R⁴, R⁵, R⁶, and R⁷ are hydrogen.

In a further aspect of the invention, a compound of the Formula Ia is employed wherein R¹ and R² form a heterocyclic, in particular a saturated 3 to 6-membered heteromonocylic group containing 1 to 4 nitrogen atoms, in particular a 6-membered ring comprising 1 or 2 nitrogen atoms [e.g., piperidinyl and piperazinyl] which may be substituted for example with alkyl, halo, carboxyl, or alkoxy carbonyl, and optionally R³, R⁴, R⁵, R⁶, and R⁷ are hydrogen.

In a further aspect of the invention, a compound of the Formula Ia is employed wherein R¹ and R⁷ form a heteroaryl, in particular an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, or pyridazinyl, R² is hydrogen or alkyl, and R³, R⁴, R⁵, R⁶, and R⁷ are hydrogen.

In a further aspect of the invention, a compound of the Formula Ia is employed wherein R¹ is silyl which may be substituted, in particular substituted with alkyl, R² is alkyl, and R³, R⁴, R⁵, R⁶, and R⁷ are hydrogen.

In some aspects, one or more of the following compounds are not within the scope of a pyridazine compound of the formula Ia or Ib employed in the present invention:
a) a compound wherein when R¹ is ═O, R³ is —COOCH₃, CH═CHCOOCH₃, —CH═CHC(═O)-phenyl, —CH═CH(C(═O)OCH₃)₂, —S-phenyl, —CH═CH(COCH₃)(COOCH₃), CH═CH(COOCH₂CH₃)₂, -phenyl-COOCH₃, —CH═CHCO-phenyl, —CH₂CH(Cl)(CH₂OH), -methylphenyl, R⁷ is hydrogen or —CH₂OCH₃, and R¹, R², R⁴, R⁵ and R⁶ are hydrogen;
b) a compound wherein when R¹ is ═O, R² is cyano, R³ is —C(═O)OCH₃, and R³, R⁴, R⁵, and R⁶ are hydrogen;
c) a compound wherein when R¹ is ═O, R² is methylthiophene or benzyl, R³, R⁴, R⁵, R⁶, and R⁷ are hydrogen;
d) a compound wherein when R¹ is ═O, R² is methyl, R⁵ is hydrogen, hydroxyl, chloro, or bromo, R⁷ is hydrogen or ethylmorpholinyl, and R³, R⁴, and R⁶ are hydrogen;
e) a compound wherein when R² is methyl, R⁵ is chloro, bromo, or hydrogen, R⁷ is hydrogen or —CH₂CH₂-morpholinyl, and R¹, R³, R⁴, and R⁶ are hydrogen;
f) a compound wherein when R¹ is piperazinyl, piperazinyl substituted with pyridinyl, phenyl, or methyl, R² is hydrogen or methyl, and R³, R⁴, R⁵, and R⁶ are hydrogen;
g) a compound wherein when R¹ is chloro or bromo, R² is C₁-C₃ alkyl, phenyl, amino, benzyl, morpholinyl, chloro, —C(═O)NH₂, —NH₂, C₁-C₃ alkylphenyl, —CH(CH₃)₂, —CH₂CH(CH₃)₂, -benzylchloro, and R³, R⁴, R⁵ and R⁶ are hydrogen;
h) a compound wherein when R¹ is chloro or bromo, R³ is hydroxyl, chloro, bromo, C₁-C₃ alkyl, phenyl, or —N(CH₃)₂, and R², R⁴, R⁵ and R⁶ are hydrogen;
i) a compound wherein when R¹ is chloro, R² is methyl, R⁵ is hydroxyl, and

R³, R⁴, and R⁶ are hydrogen;
j) a compound wherein when R¹ is chloro, R², R³, R⁴, R⁵ and R⁶ are hydrogen;
k) a compound wherein when R¹ is hydroxyl, R² is C₁-C₄ alkyl, and R³, R⁴, R⁵ and R⁶ are hydrogen;
l) a compound wherein when R¹ is —C₁-C₄ alkoxy, or C₁-C₄ alkoxy substituted with —N(CH₃)₂, morpholinyl, or piperidinyl substituted with benzyl, R² is hydrogen or methyl, R³, R⁴, R⁵ and R⁶ are hydrogen, R⁷ is absent, hydrogen, or methyl;
m) a compound wherein when R¹ is —SH, —SCH₃, or —SCH₂C(═O)CH₃, R² is hydrogen or methyl, and R³, R⁴, R⁵ and R⁶ are hydrogen;
n) a compound wherein when R¹ is ═S, R² is hydrogen or methyl, R⁷ is methyl or benzyl, and R³, R⁴, and R⁶ are hydrogen;
o) a compound wherein when R¹ is ═S, R² is methyl and R⁵ is chloro or R⁷ is methyl, and R³, R⁴, and R⁶ are hydrogen;
p) a compound wherein when R¹ is hydroxyl, R² is hydrogen, methyl, or butyl, and R³, R⁴, R⁵ and R⁶ are hydrogen;
q) a compound wherein when R¹ is methoxy, R², R³, R⁴, R⁵ and R⁶ are hydrogen;
r) a compound wherein when R¹ is C₁-C₂ alkoxy or C₁-C₄ alkoxy substituted with morpholinyl, —N(CH₃)₂, or piperidinyl substituted with benzyl, R² is methyl, and R³, R⁴, R⁵ and R⁶ are hydrogen;
s) a compound wherein R², R³, R⁴, R⁵ and R⁶ are hydrogen;
t) a compound wherein R¹ is cyano or cyano substituted with —C(OCH₂CH₃)₂, —CH(OH)(CH₃), —Si(CH₂CH₃)₂, cyclohexol, —CH₂O-trimethyldiphenylsilyl or cyclohexyl substituted with hydroxyl, and R³, R⁴, R⁵ and R⁶ are hydrogen;
u) a compound wherein R¹ is cyano substituted with —CH(OH)(CH₃)₂, —Si(CH₂CH₃)₂, morpholinyl, trimethyldiphenylsilyl, or —CH(OCH₂CH₃)₂, R² is methyl, and R³, R⁴, R⁵ and R⁶ are hydrogen;
v) a compound wherein R⁷ is oxy, and R² is hydrogen or methyl, and R³, R⁴, R⁵ and R⁶ are hydrogen;
w) a compound wherein R¹ is methyl, and R², R³, R⁴, R⁵ and R⁶ are hydrogen;
x) a compound wherein R² is methyl, and R¹, R³, R⁴, R⁵ and R⁶ are hydrogen;
y) a compound wherein R¹ is methoxycarbonyl, R³ is hydrogen, and R², R⁴, R⁵ and R⁶ are hydrogen;
z) a compound wherein R¹ is —NH₂, R² is methyl, chlorophenyl, methoxyphenyl, ethylphenyl, ethylmethoxyphenyl, propylphenyl, or —CH(CH₃)₂, R⁴, R⁵ and R⁶ are hydrogen, and R⁷ is absent or —CH₂CH₂CH₂COOH;
aa) a compound wherein R¹ is —OR²⁹ wherein R²⁹ is ethylmorpholinyl or —CH₂CH₂N(CH₃)₂ and R², R³, R⁴, R⁵ and R⁶ are hydrogen;
bb) a compound wherein R¹ is —NH₂, R³ is —NH₂, and R³, R⁴, R⁵ and R⁶ are hydrogen;
cc) a compound wherein R¹ is —NH₂, R⁵ and R⁶ are methoxy, and R³ and R⁴ are hydrogen;

dd) a compound wherein R¹ is —NH₂, R³ is methyl and R⁴, R⁵ and R⁶ are hydrogen;
ee) a compound wherein R¹ is —NH₂ , R⁵ is chloro, and R³, R⁴ and R⁶ are hydrogen;
ff) a compound wherein R¹ is —NH-chlorophenyl, and R² and R³ form a phenyl group, and R⁴, R⁵ and R⁶ are hydrogen;
gg) a compound wherein R¹ is —NH₂, R⁴ and R⁵ is methoxy, and R², R³ and R⁶ are hydrogen;
hh) a compound wherein R¹ is —NH₂, R² is ethylmethoxyphenyl, R⁷ is carboxyethyl or carboxypropyl, and R³, R⁴ and R⁶ are hydrogen;
ii) a compound wherein R¹ is —NHR⁴⁸ wherein R⁴⁸ is ethylmorpholinyl, ethylmorpholinyl substituted with ═O, —CH₂CH₂OCH₃, —CH₂CH₂CH₂CH₂CH₂CH₂CH₂CH₃, —CH₂CH₂CH₂CH₂OH, —CH₂CH₂OH, or —CH₂CH₂OCH₃, R² is hydrogen, methyl, ethyl, —CHO, —CH₂OH, —COOH, chloro, —CH₂CH₂NH₂, —NO₂, —C≡N, —C(═O)OCH₂CH₃, or —C(═O)NH₂, and R³, R⁴, R⁵ and R⁶ are hydrogen;
jj) a compound wherein R¹ is —NHR⁴⁹ wherein R⁴⁹ is ethanol, methylpiperidinylbenzyl, ethylpiperidinyl, ethylpiperidinylbenzyl, or butylpiperidinylbenzyl, R² is hydrogen, methyl, or —C(CH₃)₂, and R³, R⁴, R⁵ and R⁶ are hydrogen;
kk) a compound wherein R¹ is —NHR⁵⁵ wherein R⁵⁵ is hydrogen, and R³, R⁴, R⁵ and R⁶ are hydrogen;
ll) a compound wherein R¹ is —NHR⁵⁶ wherein R⁵⁶ is —CH₂CH₂N(CH₂CH₃)₂ or ethylmorpholinyl, R³ is ethyl, and R⁴, R⁵ and R⁶ are hydrogen;
mm) a compound wherein R¹ is —NHNH₂, R³ is hydrogen, alkyl, or phenyl, and R³, R⁴, R⁵ and R⁶ are hydrogen;
nn) a compound wherein R¹ is —NHR⁵⁷ wherein R⁵⁷ is NH₂, —CH₂CH₂OH, CH₂CH(OH)(CH₃), ethylmorpholinyl, ethylmorpholinyl substituted with ═O, ethylphenyl, —CH₂CH₂NHCH₃, —CH₂CH₂N(—CH₂CH₂CH₃)₂, ethylpiperidinyl, or ethylpiperidinylbenzyl, R² is methyl, and R³, R⁴, R⁵ and R⁶ are hydrogen;
oo) a compound wherein R¹ is morpholinyl, R² is —C(F)₃, —C(═O), —CH₂OH, —C(═O)H, —COOH, chloro, —NO₂, or cyano, and R³, R⁴, R⁵ and R⁶ are hydrogen;
pp) a compound wherein R¹ is —NHR⁵⁸ wherein R⁵⁸ is heptyl, phenyl, benzyl, or ethylphenyl, R² is hydrogen, methyl, or chlorophenyl, R⁴, R⁵ and R⁶ are hydrogen;
qq) a compound wherein R¹ is —NR⁹ wherein R⁹ is phenyl and R², R³, R⁴, R⁵ and R⁶ are hydrogen;
rr) a compound wherein R¹ is morpholinyl and R², R³, R⁴, R⁵ and R⁶ are hydrogen;
ss) a compound wherein R¹ is methylpiperazinyl and R², R³, R⁴, R⁵ and R⁶ are hydrogen;
tt) a compound wherein R¹ is —NHCH₂CH₂OH or NHCH₂CH₂OCH₃, R² is phenyl and R³, R⁴, R⁵ and R⁶ are hydrogen;
uu) a compound wherein R¹ is —NHR⁵⁹ wherein R⁵⁹ is ethylamino, butylamino, ethylaminomethyl, and R² is hydrogen, methyl, or —C(═O)NH₂, and R³, R⁴, R⁵ and R⁶ are hydrogen;
vv) a compound wherein R¹ is —NHR⁶⁰ wherein R⁶⁰ is ethylpiperidinyl, methylpiperidinylbenzyl, piperidinylbenzyl, ethylpiperidinylbenzyl, methylpyrrolidinylmethyl, ethylpiperazinylbenzyl, —CH₂C(═O)-piperazinylbenzyl, —C(═O)-methylnaphthyl, —CH₂CH₂CH₂CH₂CH₂N(CH₃)(C₇H₇), —CH₂C(═O)-piperidinylbenzyl, —C(═O)-methylpiperidinylbenzyl, or —CH(CH₃)₂, and R³, R⁴, R⁵ and R⁶ are hydrogen;
ww) a compound wherein R¹ is —CHCH₂CH₂-isoquinolinyl, —NHCH₂CH₂N(CH₂CH₂CH₃)₂, propyl substituted with piperidinyl fused to phenyl, —NHCH₂CH₂, or —NHCH₂CH₂CH₂CH₂CH₂ substituted with a piperidinyl fused to two adjacent carbon atoms of a phenyl moiety;
xx) a compound wherein R¹ is —NH substituted with two pyrrolidinyl groups; R³ is methyl, and R², R⁴, R⁵ and R⁶ are hydrogen;
yy) a compound wherein R¹ is —COOCH₃, R³ is methyl, and R², R⁴, R⁵ and R⁶ are hydrogen;
zz) a compound wherein R¹ is hydrogen, R² is methyl, R⁷ is oxygen;
aaa) a compound wherein R⁷ is methyl or oxygen, and R¹,R², R⁴, R⁵ and R⁶ are hydrogen;
bbb) a compound wherein R¹ is —NHCH₂CH₂N(CH₂CH₃)₂, R³ is ethyl, and R², R⁴, R⁵ and R⁶ are hydrogen; and
ccc) a compound wherein R¹ is —NHCH₂CH(OH)(CH₃) or —NHCH₂CH₂NHCH₂CH₂OH, R² is methyl, and R³, R⁴, R⁵ and R⁶ are hydrogen.

In aspects of the invention a compound of the formula Ia or Ib is employed wherein R¹ is a piperazinyl or substituted piperazinyl, in particular a piperazinyl substituted with a pyrimidinyl of Formula A below.

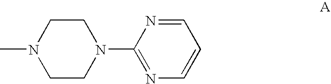

Thus, a pyridazine compound for use in the present invention includes compounds of the Formula II:

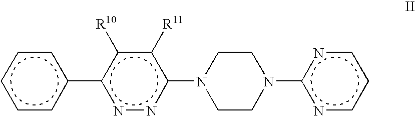

wherein R¹⁰ and R¹¹ are independently substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, phosphonate, cyano, halo, silyl, silyloxy, silylalkyl, silylthio, ═O, ═S, carboxyl, carbonyl, carbamoyl, or carboxamide; or an isomer or a pharmaceutically acceptable salt thereof.

In an aspect of the invention, a compound of the Formula II is employed wherein R¹⁰ is hydrogen; hydroxyl; alkyl; aryl [e.g. phenyl which is optionally substituted (e.g., halide)]; piperazinyl which may be substituted (e.g. substituted with a pyrimidinyl); —NR³⁶R³⁷ wherein R³⁶ is hydrogen or alkyl, and R³⁷ is phenyl which may be substituted or alkyl which may be substituted (e.g. amino, in particular —CH₂CH₂NH₂; CH₂CH₂NHCOOC(CH₃)₃); morpholinyl which may be substituted; or —SR²³ wherein R²³ is phenyl which may be substituted; and R¹¹ is hydrogen, or aryl (e.g. phenyl) which may be substituted.

In a particular aspect of the invention a compound of the Formula II is employed wherein R¹⁰ is hydrogen, halo, optionally substituted hydroxyl, alkyl, pyridinyl, phenyl, benzyl, piperazinyl, amino, morpholinyl, or —SR²⁴ wherein R²⁴ is alkyl or aryl. In an embodiment, R¹⁰ is —NH[CH₂]ₘNR⁶¹R⁶² wherein m is 1 to 6, in particular 2 to 4, R⁶¹ is hydrogen, R⁶² is a carboxyl, in particular —COOC(CH₃)₃.

In an aspect of the invention, a compound of the Formula II is employed wherein R¹¹ is hydrogen, halo, optionally substituted alkyl, pyridinyl, piperidinyl, morpholinyl, piperazinyl, or phenyl.

In another aspect of the invention, a compound of the Formula II is employed wherein both of R¹⁰ and R¹¹ are not hydrogen.

In particular embodiments of the invention, one or more of R¹⁰ and R¹¹ are alkyl, in particular C₁-C₆ alkyl and the other of R¹⁰ and R¹¹ is hydrogen.

In particular embodiments of the invention, one or more of R¹⁰ and R¹¹ are aryl in particular phenyl or benzyl and the other of R¹⁰ and R¹¹ is hydrogen.

In particular embodiments of the invention, a compound of the Formula II is a compound in Table 3, more particularly a compound designated MW01-2-065LKM, MW01-2-069SRM, MW01-2-151SRM, MW01-5-188WH, MW01-6-127WH, MW01-6-189WH, or MW01-7-107WH, and pharmaceutically acceptable salts, and derivatives thereof.

In aspects, the invention employs a compound of the Formula III:

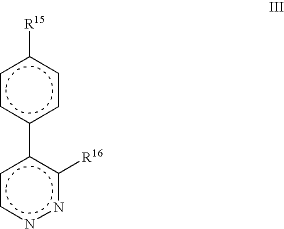

wherein R¹⁵ and R¹⁶ are independently substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide; or an isomer or a pharmaceutically acceptable salt thereof.

In other aspects, the invention employs a compound of the Formula IV:

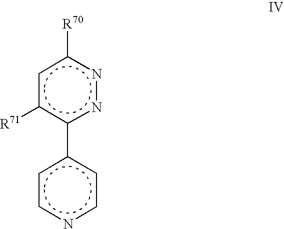

wherein R⁷⁰ and R⁷¹ are independently substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silyalkyl, silylthio, ═O, ═S, phosphonate, carboxyl, carbonyl, or carbamoyl, or an isomer or pharmaceutically acceptable salt thereof.

In other aspects, a compound of the formula IV is employed wherein R⁷⁰ is substituted or unsubstituted hydrogen, hydroxyl, alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, halo, silyl, silyloxy, silylalkyl, silylthio, ═O, ═S, carboxyl, carbonyl, carbamoyl, or carboxamide, especially heterocyclic, heteroaryl, amino, and substituted amino and R⁷¹ is aryl or substituted aryl; or an isomer or a pharmaceutically acceptable salt thereof.

In another aspect, a compound of the Formula IV is employed wherein R⁷⁰ is a heterocylic, in particular a saturated 3 to 6-membered heteromonocylic group containing 1 to 4 nitrogen atoms more particularly, pyrrolidinyl, imidazolidinyl, piperidinyl, and piperazinyl, especially piperazinyl or piperidinyl, which may be substituted with alkyl especially methyl, dimethyl, cycloalkyl especially cyclohexyl, aryl especially phenyl, a substituted or unsubstituted unsaturated condensed heterocyclic group containing 1 to 5 nitrogen atoms, in particular, indolyl, isoindolyl, indolizinyl, indazolyl, quinazolinyl, pteridinyl, quinolizidinyl, phthalazinyl, naphthyridinyl, quinoxalinyl, cinnolinyl, phenanthridinyl, acridinyl, phenanthrolinyl, phenazinyl, carbazolyl, purinyl, benzimidazolyl, quinolyl, isoquinolyl, quinolinyl, isoquinolinyl, or indazolyl, especially benzimidazolyl substituted with oxy.

In other aspects, a compound of the Formula IV is employed wherein R⁷⁰ is amino or substituted amino, and optionally R⁷¹ is aryl, in particular phenyl. In an aspect R⁷⁰ is —N—R⁶³ wherein R⁶³ is hydrogen or alkyl, in particular C₁-C₆ alkyl, more particularly methyl or dimethyl, or —N—R⁴⁰R⁴¹ wherein R⁴⁰ is hydrogen or alkyl, in particular C₁-C₆ alkyl, more particularly methyl and R⁴¹ is alkyl substituted with amino or substituted amino, heterocyclic, substituted heterocylic, or cycloalkyl. In an embodiment, R⁷⁰ is —N—R⁴²R⁴³ wherein R⁴² is hydrogen or alkyl, in particular C₁-C₆ alkyl, more particularly methyl and R⁴³ is C₁-C₆ alkyl, especially methyl or ethyl substituted with a cycloalkyl especially cyclopropyl, a heterocyclic especially piperidinyl, pyrrolidinyl, or morpholinyl which may be substituted in particular substituted with aryl, especially benzyl.

A compound of the Formula IV may comprise a structure designated as compound 124, 125, 126, 127, 128, 129, 130, 131, 132, 133, 134, 135, 136, 137, 138, or 139 in Table 5 or pharmaceutically acceptable salts, isomers, or derivatives thereof.

In further aspects, the invention employs a compound of the Formula V:

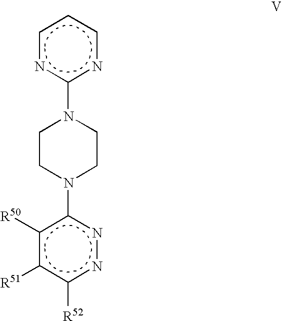

wherein R⁵⁰, R⁵¹, and R⁵² are independently substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, carboxyl, carbonyl, carbamoyl, or carboxamide; or an isomer or a pharmaceutically acceptable salt thereof.

In aspects of the invention a compound of the Formula V is employed wherein R⁵⁰ is substituted or unsubstituted hydrogen, alkyl, aryl, or heterocyclic; R⁵¹ is substituted or unsubstituted hydrogen or alkyl, and R⁵² is substituted or unsubstituted hydrogen, alkyl, cycloalkyl, heteroaryl or halo. In an aspect, a compound of the Formula V is employed wherein R⁵⁰ is hydrogen, C₁-C₆ alkyl which may be substituted with alkyl, especially methyl or trimethyl, phenyl, or a 3 to 6-membered heteromonocylic group containing 1 to 4 nitrogen atoms more particularly, piperidinyl or morpholinyl, R⁵¹ is hydrogen or alkyl especially methyl, and R⁵² is hydrogen, alkyl especially methyl, dimethyl, ethyl, or propyl, cyclohexyl, chloro, or an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, or pyridazinyl, especially pyridinyl. In an embodiment, R⁵⁰ is aryl, R⁵¹ is hydrogen, and R⁵² is C₁-C₆ alkyl.

A compound of the Formula V may comprise compound MW01-7-057WH, or structure 32, 34, 36, 38, 39, 40, 41, 42, 43, 44, 46, 47, 48, 49, 63, 69, 70, 71, 75, 76, 77, 78, 79, 80, 81, or 82 in Table 5 or pharmaceutically acceptable salts, isomers or derivatives thereof.

In aspects of the invention the pyridazine compound is an isolated and pure, in particular, substantially pure, compound of the Formula I, II, III, IV, or V, or an isomer or a pharmaceutically acceptable salt thereof. As used herein, the term “pure” in general means better than 90%, 92%, 93%, 94%, 95%, 96%, 97%, 98%, or 99% pure, and “substantially pure” means a compound synthesized such that the compound, as made or as available for consideration into a composition or dosage form described herein, has only those impurities that can not readily nor reasonably be removed by conventional purification processes.

A pyridazine compound employed in the invention includes derivatives, in particular derivatives of a compound of the Formula I, II, III, IV, or V. The term “derivative” of a compound, as used herein, refers to a chemically modified compound wherein the chemical modification takes place either at a functional group of the compound or on the aromatic ring. Non-limiting examples of derivatives of compounds of the Formula I, II, III, IV, or V (e.g., pyridazine derivatives of the present invention) may include N-acetyl, N-methyl, N-hydroxy groups at any of the available nitrogens in the compound. Derivative groups that may be used to modify the compounds of the Formula I, II, III, IV, or V can be found in U.S. Patent Application No. 20030176437 (herein incorporated by reference in its entirety for all purposes).

In some embodiments, the organic compounds, and/or heterocyclic derivatives thereof depicted in Tables 1, 2, 3, 4 or 5 are employed, in particular Tables 2, 3, 4, or 5.

In particular aspects the invention employs a compound of the Formula I, II, III, IV, or V as defined herein, with the proviso that compounds depicted in Table 1 are excluded.

In other particular aspects the invention employs a compound of the Formula II with the proviso that the compounds depicted in Table 1 are excluded.

In further particular aspects the invention employs a compound of the Formula III with the proviso that compounds depicted in Table 1 are excluded.

In further particular aspects the invention employs compounds of the Formula IV with the proviso that compounds depicted in Table 1 are excluded.

In still further particular aspects the invention employs compounds of the Formula V with the proviso that compounds depicted in Table 1 are excluded.

In accordance with aspects of the invention pyridazine compounds and/or related heterocyclic derivatives thereof (see, for example, the Tables herein, in particular Table 2, 3, 4 and/or 5 or heterocyclic derivatives thereof), are employed in the treatment or prevention of disorders disclosed herein. In some embodiments, the compounds employed are those depicted in Table 2, 3, 4, and/or 5 or derivatives thereof. In some embodiments, the invention employs one or more of the compounds designated herein as MW01-3-183WH, MW01-5-188WH, MW01-2-065LKM, MW01-2-184WH, MW01-2-189WH and MW01-2-151SRM, or isomers or pharmaceutically acceptable salts thereof.

In some embodiments, the invention employs one or more of the compounds designated herein as MW01-3-183WH, MW01-5-188WH, MW01-2-065LKM, MW01-2-184WH, MW01-2-189WH and MW01-2-151SRM, or isomers or pharmaceutically acceptable salts thereof.

In some embodiments, the invention employs one or more of the compounds designated MW01-3-183WH, MW01-5-188WH, MW01-2-065LKM, MW01-2-184WH, MW01-2-151SRM, MW01-2-189WH, and MW01-1-01-L-D07, and/or related derivatives, in particular, heterocyclic derivatives, of these compounds. In another particular embodiment of the invention, MW01-2-151SRM, an isomer, a pharmaceutically acceptable salt, or derivative thereof is employed in the invention. In a particular embodiment of the invention, MW01-5-188WH, an isomer, a pharmaceutically acceptable salt, or derivative thereof is employed in the invention.

A pyridazine compound also includes “pharmaceutically acceptable salt(s)”. By pharmaceutically acceptable salts is meant those salts which are suitable for use in contact with the tissues of a subject or patient without undue toxicity, irritation, allergic response and the like, and are commensurate with a reasonable benefit/risk ratio. Pharmaceutically acceptable salts are described for example, in S. M. Berge, et al., J. Pharmaceutical Sciences, 1977, 66:1. Examples of salts include the compounds designated herein as MW01-1-01-L-D10, MW01-1-01-L-E02, MW01-1-01-L-E08, MW01-1-03-L-A05, MW01-1-16-L-D09, and MW01-1-17-L-G04.

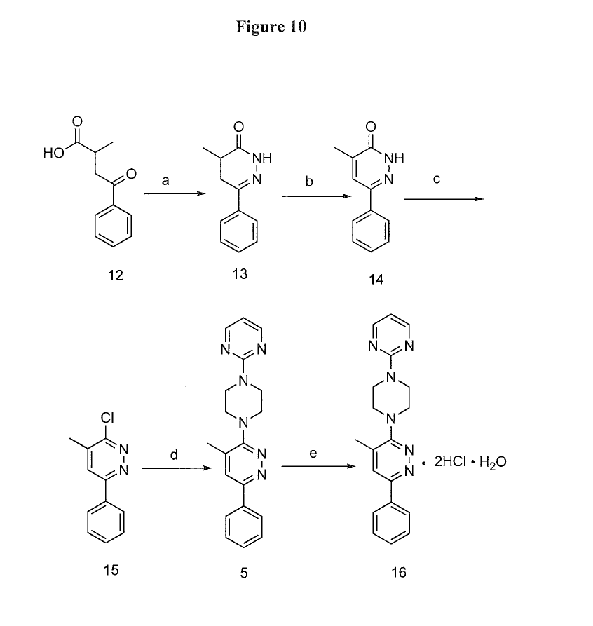

In aspects of the invention, an acid addition salt, in particular a halide salt, more particularly a chloride salt, most particularly a hydrochloride salt of a compound of the formula II is employed. In a particular embodiment, a pharmaceutically acceptable halide salt of the pyridazine compound 4-methyl-6-phenyl-3-(4-pyrimidin-2-ylpiperazin-1-yl)pyridazine(5) shown in FIG. 10 is employed.

In an embodiment, a pharmaceutically acceptable salt employed in the invention is a chloride salt of 4-methyl-6-phenyl-3-(4-pyrimidin-2-ylpiperazin-1-yl)pyridazine(5) shown in FIG. 10. In a particular embodiment, a pharmaceutically acceptable salt is a hydrochloride salt of 4-methyl-6-phenyl-3-(4-pyrimidin-2-ylpiperazin-1-yl)pyridazine (5) shown in FIG. 10, more particularly the di-hydrochloride hydrate salt shown below (i.e., MW01-9-034WH)(16)(i.e., 2-(4-(4-methyl-6-phenylpyridazin-3-yl)piperazin-1-yl)pyrimidine dihydrochloride salt)(6).

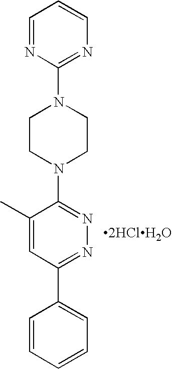

In aspects of the invention a di-hydrochloride hydrate salt of a compound of the Formula V, in particular 2-(4-(4-methyl-6-phenylpyridazin-3-yl)piperazin-1-yl)pyrimidine dihydrochloride salt (6) shown in FIG. 10, characterized by enhanced solubility is utilized in the present invention. The di-hydrochloride hydrate salt may be further characterized by one or more of the following: a yellow powder, having a purity of greater than about 90%, 92%, 93%, 94%, 95%, 96%, 97%, 98%, or 99%, molecular weight of 423.3395, soluble in DMSO, melting point of greater than 488K, acid dissociation constants (pKa) of 2.55, 1.46, 0.84 and −4.31 (calculated), logP of 2.29 determined from octanol/water partition coefficient, logS of 2.5 (experimental)/4.08 (calculated), and/or having an aqueous solubility at 37° C. of about 100 to 400 mg/ml, about 100 to 350 mg/ml, about 150 to 350 mg/ml, about 200 to 350 mg/ml, or about 300 to 350 mg/ml, in particular a solubility of greater than 322 mg/ml in water (2HCL.H₂O) salt).

In aspects, a compound of the Formula V in amorphous or crystalline form that has an enhanced resorption rate is utilized. In particular aspects the resorption rate is increased by a factor of at least 2, 3, 4 or 5.

A pyridazine compound, in particular a compound of the Formula I, II, III, IV, or V, may contain one or more asymmetric centers and may give rise to enantiomers, diasteriomers, and other stereoisomeric forms which may be defined in terms of absolute stereochemistry as (R)- or (S)-. Thus, pyridazine compounds include all possible diasteriomers and enantiomers as well as their racemic and optically pure forms. Optically active (R)- and (S)-isomers may be prepared using chiral synthons or chiral reagents, or resolved using conventional techniques. When a pyridazine compound contains centers of geometric asymmetry, and unless specified otherwise, it is intended that the compounds include both E and A geometric isomers. All tautomeric forms are also included within the scope of a pyridazine compound employed in the present invention.

A compound of the formula I, II, III, IV or V includes crystalline forms which may exist as polymorphs. Solvates of the compounds formed with water or common organic solvents are also intended to be encompassed within the term. Thus, a pyridazine compound, in particular a compound of the Formula I, II, III, IV, or V, can exist in unsolvated as well as solvated forms with pharmaceutically acceptable solvents such as water, ethanol, and the like. The solvated forms may be considered equivalent to the unsolvated forms for the purposes of the present invention. In addition, hydrate forms of the compounds and their salts are encompassed within this invention. Further prodrugs of compounds of the formula I, II, III, IV or V are encompassed within the term.

The term “solvate” means a physical association of a compound with one or more solvent molecules or a complex of variable stoichiometry formed by a solute (for example, a compound of the invention) and a solvent, for example, water, ethanol, or acetic acid. This physical association may involve varying degrees of ionic and covalent bonding, including hydrogen bonding. In certain instances, the solvate will be capable of isolation, for example, when one or more solvent molecules are incorporated in the crystal lattice of the crystalline solid. In general, the solvents selected do not interfere with the biological activity of the solute. Solvates encompass both solution-phase and isolatable solvates. Representative solvates include hydrates, ethanolates, methanolates, and the like.

Dehydrate, co-crystals, anhydrous, or amorphous forms of the compounds of the invention are also included. The term “hydrate” means a solvate wherein the solvent molecule(s) is/are H₂O, including, mono-, di-, and various poly-hydrates thereof. Solvates can be formed using various methods known in the art.

Crystalline compounds of the formula I, II, III, IV or V can be in the form of a free base, a salt, or a co-crystal. Free base compounds can be crystallized in the presence of an appropriate solvent in order to form a solvate. Acid salt compounds of the formula I, II, III, IV or V (e.g. HC1, HBr, benzoic acid) can also be used in the preparation of solvates. For example, solvates can be formed by the use of acetic acid or ethyl acetate. The solvate molecules can form crystal structures via hydrogen bonding, van der Waals forces, or dispersion forces, or a combination of any two or all three forces.

The amount of solvent used to make solvates can be determined by routine testing. For example, a monohydrate of a compound of the formula I, II, III, IV or V would have about 1 equivalent of solvent (H₂O) for each equivalent of a compound of the invention. However, more or less solvent may be used depending on the choice of solvate desired.

Compounds of the formula I, II, III, IV or V may be amorphous or may have different crystalline polymorphs, possibly existing in different solvation or hydration states. By varying the form of a drug, it is possible to vary the physical properties thereof. For example, crystalline polymorphs typically have different solubilities from one another, such that a more thermodynamically stable polymorph is less soluble than a less thermodynamically stable polymorph. Pharmaceutical polymorphs can also differ in properties such as shelf-life, bioavailability, morphology, vapor pressure, density, color, and compressibility.

A compound of the Formula I, II, III, IV, or V may be in the form of a prodrug that is converted in vivo to an active compound. In a compound of the Formula I one or more of R¹, R², R³, R⁴, R⁵, R⁶, and R⁷ may comprise a cleavable group that is cleaved after administration to a subject to provide an active (e.g., therapeutically active) compound, or an intermediate compound that subsequently yields the active compound. A cleavable group can be an ester that is removed either enzymatically or non-enzymatically.

The term “prodrug” means a covalently-bonded derivative or carrier of the parent compound or active drug substance which undergoes at least some biotransformation prior to exhibiting its pharmacological effect(s). In general, such prodrugs have metabolically cleavable groups and are rapidly transformed in vivo to yield the parent compound, for example, by hydrolysis in blood, and generally include esters and amide analogs of the parent compounds. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al. (eds.), Vol. 42, Academic Press, 1985, particularly pp. 309 396; Burger's Medicinal Chemistry and Drug Discovery, 5th Ed., M. Wolff (ed.), John Wiley & Sons, 1995, particularly Vol. 1 and pp. 172 178 and pp. 949 982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987.

Examples of prodrugs include, but are not limited to esters (e.g., acetate, formate, and benzoate derivatives), carbamates (e.g. N,N-dimethylaminocarbonyl) of hydroxy functional groups on compounds of the formula I, II, III, IV or V, and the like.

A compound of the formula I, II, III, IV or V compound can include a pharmaceutically acceptable co-crystal or a co-crystal salt. A pharmaceutically acceptable co-crystal includes a co-crystal that is suitable for use in contact with the tissues of a subject or patient without undue toxicity, irritation, allergic response and has the desired pharmacokinetic properties.

The term “co-crystal” as used herein means a crystalline material comprised of two or more unique solids at room temperature, each containing distinctive physical characteristics, such as structure, melting point, and heats of fusion. Co-crystals can be formed by an active pharmaceutical ingredient (API) and a co-crystal former either by hydrogen bonding or other non-covalent interactions, such as pi stacking and van der Waals interactions. An aspect of the invention provides for a co-crystal wherein the co-crystal former is a second API. In another aspect, the co-crystal former is not an API. In another aspect, the co-crystal comprises more than one co-crystal former. For example, two, three, four, five, or more co-crystal formers can be incorporated in a co-crystal with an API. Pharmaceutically acceptable co-crystals are described, for example, in “Pharmaceutical co-crystals,” Journal of Pharmaceutical Sciences, Volume 95 (3) Pages 499-516, 2006. Methods for producing co-crystals are discussed in the United States Patent Application 20070026078.

A co-crystal former which is generally a pharmaceutically acceptable compound, may be, for example, benzoquinone, terephthalaldehyde, saccharin, nicotinamide, acetic acid, formic acid, butyric acid, trimesic acid, 5-nitroisophthalic acid, adamantane-1,3,5,7-tetracarboxylic acid, formamide, succinic acid, fumaric acid, tartaric acid, malic acid, malonic acid, benzamide, mandelic acid, glycolic acid, fumaric acid, maleic acid, urea, nicotinic acid, piperazine, p-phthalaldehyde, 2,6-pyridinecarboxylic acid, 5-nitroisophthalic acid, citric acid, and the alkane- and arene-sulfonic acids such as methanesulfonic acid and benezenesulfonic acid.

In general, all physical forms of compounds of the formula I, II, III, IV or V are intended to be within the scope of the present invention.

A pyridazine compound, in particular a compound of the Formula I, II, III, IV, or V, may optionally comprise a carrier interacting with one or more radicals in the compound, for example R¹, R², R³, R⁴, R⁵, R⁶ or R⁷ in Formula I. A carrier may be a polymer, carbohydrate, or peptide, or derivatives or combinations thereof, and it may be optionally substituted, for example, with one or more alkyl, halo, hydroxyl, halo, or amino. A carrier may be directly or indirectly covalently attached to a pyridazine compound. A carrier may be substituted with substituents described herein including without limitation one or more alkyl, amino, nitro, halogen, thiol, thioalkyl, sulfate, sulfonyl, sulfinyl, sulfoxide and hydroxyl groups. In aspects of the invention the carrier is an amino acid including alanine, glycine, praline, methionine, serine, threonine, asparagine, alanyl-alanyl, prolyl-methionyl, or glycyl-glycyl. A carrier can also include a molecule that targets a pyridazine compound, in particular a compound of the Formula I, II, III, IV, or V, to a particular tissue or organ. Thus, a carrier may facilitate or enhance transport of a pyridazine compound, in particular a compound of the Formula I, II, III, IV or V, to a target therapeutic site, for example the brain.

A “polymer” refers to molecules comprising two or more monomer subunits that may be identical repeating subunits or different repeating subunits. A monomer generally comprises a simple structure, low-molecular weight molecule containing carbon. Polymers may optionally be substituted. Polymers that can be used in the present invention include without limitation vinyl, acryl, styrene, carbohydrate derived polymers, polyethylene glycol (PEG), polyoxyethylene, polymethylene glycol, poly-trimethylene glycols, polyvinylpyrrolidone, polyoxyethylene-polyoxypropylene block polymers, and copolymers, salts, and derivatives thereof. In aspects of the invention, the polymer is poly(2-acrylamido-2-methyl-1-propanesulfonic acid); poly(2-acrylamido-2-methyl,-1-propanesulfonic acid-coacrylonitrile, poly(2-acrylamido-2-methyl-1-propanesulfonic acid-co-styrene), poly(vinylsulfonic acid); poly(sodium 4-styrenesulfonic acid); and sulfates and sulfonates derived therefrom; poly(acrylic acid), poly(methylacrylate), poly(methyl methacrylate), and poly(vinyl alcohol).

A “carbohydrate” as used herein refers to a polyhydroxyaldehyde, or polyhydroxyketone and derivatives thereof. The term includes monosaccharides such as erythrose, arabinose, allose, altrose, glucose, mannose, threose, xylose, gulose, idose, galactose, talose, aldohexose, fructose, ketohexose, ribose, and aldopentose. The term also includes carbohydrates composed of monosaccharide units, including disaccharides, ogliosaccharides, or polysaccharides. Examples of disaccharides are sucrose, lactose, and maltose. Oligosaccharides generally contain between 3 and 9 monosaccharide units and polysaccharides contain greater than 10 monosaccharide units. A carbohydrate group may be substituted at one two, three or four positions, other than the position of linkage to a pyridazine compound. For example, a carbohydrate may be substituted with one or more alkyl, amino, nitro, halo, thiol, carboxyl, or hydroxyl groups, which are optionally substituted. Illustrative substituted carbohydrates are glucosamine, or galactosamine. In aspects of the invention, the carbohydrate is a sugar, in particular a hexose or pentose and may be an aldose or a ketose. A sugar may be a member of the D or L series and can include amino sugars, deoxy sugars, and their uronic acid derivatives. In embodiments of the invention where the carbohydrate is a hexose, the hexose is glucose, galactose, or mannose, or substituted hexose sugar residues such as an amino sugar residue such as hexosamine, galactosamine, glucosamine, in particular D-glucosamine (2-amino-2-doexy-D-glucose) or D-galactosamine (2-amino-2-deoxy-D-galactose). Illustrative pentose sugars include arabinose, fucose, and ribose.

A sugar residue may be linked to a pyridazine compound from a 1,1 linkage, 1,2 linkage, 1,3 linkage, 1,4 linkage, 1,5 linkage, or 1,6 linkage. A linkage may be via an oxygen atom of a pyridazine compound. An oxygen atom can be replaced one or more times by —CH₂— or —S— groups.

The term “carbohydrate” also includes glycoproteins such as lectins (e.g. concanavalin A, wheat germ agglutinin, peanutagglutinin, seromucoid, and orosomucoid) and glycolipids such as cerebroside and ganglioside.

A “peptide” carrier includes one, two, three, four, or five or more amino acids covalently linked through a peptide bond. A peptide can comprise one or more naturally occurring amino acids, and analogs, derivatives, and congeners thereof. A peptide can be modified to increase its stability, bioavailability, solubility, etc. “Peptide analogue” and “peptide derivative” as used herein include molecules which mimic the chemical structure of a peptide and retain the functional properties of the peptide. A carrier can be an amino acid such as alanine, glycine, proline, methionine, serine, threonine, histidine, asparagine, alanyl-alanyl, prolyl-methionyl, or glycyl-glycyl. A carrier can be a polypeptide such as albumin, antitrypsin, macroglobulin, haptoglobin, caeruloplasm, transferring, α- or β-lipoprotein, β- or γ-globulin or fibrinogen. A peptide can be attached to a pyridazine compound through a functional group on the side chain of certain amino acids (e.g. serine) or other suitable functional groups. A carrier may comprise four or more amino acids with groups attached to three or more of the amino acids through functional groups on side chains. In an aspect, the carrier is one amino acid, in particular a sulfonate derivative of an amino acid, for example cysteic acid.

Approaches to designing peptide analogues, derivatives and mimetics are known in the art. For example, see Farmer, P. S. in Drug Design (E. J. Ariens, ed.) Academic Press, New York, 1980, vol. 10, pp. 119-143; Ball. J. B. and Alewood, P. F. (1990) J Mol. Recognition 3:55; Morgan, B. A. and Gainor, J. A. (1989) Ann. Rep. Med. Chem. 24:243; and Freidinger, R. M. (1989) Trends Pharmacol. Sci. 10:270. See also Sawyer, T. K. (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith, A. B. 3rd, et al. (1995) J. Am. Chem. Soc. 117:11113-11123; Smith, A. B. 3rd, et al. (1994) J. Am. Chem. Soc. 116:9947-9962; and Hirschman, R., et al. (1993) J. Am. Chem. Soc. 115:12550-12568.

The term “alkyl”, either alone or within other terms such as “thioalkyl” and “arylalkyl”, means a monovalent, saturated hydrocarbon radical which may be a straight chain (i.e. linear) or a branched chain. An alkyl radical for use in the present invention generally comprises from about 1 to 20 carbon atoms, particularly from about 1 to 10, 1 to 8 or 1 to 7, more particularly about 1 to 6 carbon atoms, or 3 to 6 carbon atoms. Illustrative alkyl radicals include methyl, ethyl, n-propyl, n-butyl, n-pentyl, n-hexyl, isopropyl, isobutyl, isopentyl, amyl, sec-butyl, tert-butyl, tert-pentyl, n-heptyl, n-octyl, n-nonyl, n-decyl, undecyl, n-dodecyl, n-tetradecyl, pentadecyl, n-hexadecyl, heptadecyl, n-octadecyl, nonadecyl, eicosyl, dosyl, n-tetracosyl, and the like, along with branched variations thereof. In certain aspects of the invention an alkyl radical is a C₁-C₆ lower alkyl comprising or selected from the group consisting of methyl, ethyl, n-propyl, n-butyl, n-pentyl, n-hexyl, isopropyl, isobutyl, isopentyl, amyl, tributyl, sec-butyl, tert-butyl, tert-pentyl, and n-hexyl. An alkyl radical may be optionally substituted with substituents as defined herein at positions that do not significantly interfere with the preparation of compounds of the Formula I, II, III, IV, or V and do not significantly reduce the efficacy of the compounds. In certain aspects of the invention, an alkyl radical is substituted with substituents, in particular one to five substituents, including halo, lower alkoxy, lower aliphatic, a substituted lower aliphatic, hydroxy, cyano, nitro, thio, amino, keto, aldehyde, ester, amide, substituted amino, carboxyl, sulfonyl, sulfinyl, sulfenyl, sulfate, sulfoxide, substituted carboxyl, halogenated lower alkyl (e.g. CF₃), halogenated lower alkoxy, hydroxycarbonyl, lower alkoxycarbonyl, lower alkylcarbonyloxy, lower alkylcarbonylamino, cycloaliphatic, substituted cycloaliphatic, or aryl (e.g., phenylmethyl (i.e. benzyl)). Substituents on an alkyl group may themselves be substituted.

As used herein in respect to certain aspects of the invention, the term “substituted aliphatic” refers to an alkyl or an alkane possessing less than 10 carbons where at least one of the aliphatic hydrogen atoms has been replaced by a halogen, an amino, a hydroxy, a nitro, a thio, a ketone, an aldehyde, an ester, an amide, a lower aliphatic, a substituted lower aliphatic, or a ring (aryl, substituted aryl, cycloaliphatic, or substituted cycloaliphatic, etc.). Examples of such groups include, but are not limited to, 1-chloroethyl and the like.

As used herein in respect to certain aspects of the invention, the term “lower-alkyl-substituted-amino” refers to any alkyl unit containing up to and including eight carbon atoms where one of the aliphatic hydrogen atoms is replaced by an amino group. Examples of such groups include, but are not limited to, ethylamino and the like.

As used herein in respect to certain aspects of the invention, the term “lower-alkyl-substituted-halogen” refers to any alkyl chain containing up to and including eight carbon atoms where one of the aliphatic hydrogen atoms is replaced by a halogen. Examples of such groups include, but are not limited to, chlorethyl and the like.

As used herein, the term “acetylamino” shall mean any primary or secondary amino that is acetylated. Examples of such groups include, but are not limited to, acetamide and the like.

As used herein the term “alkenyl” refers to an unsaturated, acyclic branched or straight-chain hydrocarbon radical comprising at least one double bond. An alkenyl radical may contain from about 2 to 24, 2 to 15, or 2 to 10 carbon atoms, in particular from about 3 to 8 carbon atoms and more particularly about 3 to 6 or 2 to 6 carbon atoms. Suitable alkenyl radicals include without limitation ethenyl, propenyl (e.g., prop-1-en-1-yl, prop-1-en-2-yl, prop-2-en-1-yl (allyl), and prop-2-en-2-yl), buten-1-yl, but-1-en-2-yl, 2-methyl-prop-1-en-1-yl, but-2-en-1-yl, but-2-en-2-yl, buta-1,3-dien-1-yl, buta-1,3-dien-2-yl, hexen-1-yl, 3-hydroxyhexen-1-yl, hepten-1-yl, and octen-1-yl, and the like. An alkenyl radical may be optionally substituted similar to alkyl.

In aspects of the invention, “substituted alkenyl” includes an alkenyl group substituted by, for example, one to three substituents, preferably one to two substituents, such as alkyl, alkoxy, haloalkoxy, alkylalkoxy, haloalkoxyalkyl, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

As used herein, the term “alkynyl” refers to an unsaturated, branched or straight-chain hydrocarbon radical comprising one or more triple bonds. An alkynyl radical may contain about 1 to 20, 1 to 15, or 2-10 carbon atoms, particularly about 3 to 8 carbon atoms and more particularly about 3 to 6 carbon atoms. Suitable alkynyl radicals include without limitation ethynyl, such as prop-1-yn-1-yl and prop-2-yn-1-yl, butynyls such as but-1-yn-1-yl, but-1-yn-3-yl, and but-3-yn-1-yl, pentynyls such as pentyn-1-yl, pentyn-2-yl, 4-methoxypentyn-2-yl, and 3-methylbutyn-1-yl, hexynyls such as hexyn-1-yl, hexyn-2-yl, hexyn-3-yl, and 3,3-dimethylbutyn-1-yl radicals and the like. An alkynyl may be optionally substituted similar to alkyl. The term “cycloalkynyl” refers to cyclic alkynyl groups.

In aspects of the invention, “substituted alkynyl” includes an alkynyl group substituted by, for example, a substituent, such as, alkyl, alkoxy, alkanoyl, alkanoyloxy, cycloalkyl, cycloalkoxy, acyl, acylamino, acyloxy, amino, alkylamino, alkanoylamino, aminoacyl, aminoacyloxy, cyano, halogen, hydroxyl, carboxyl, carboxylalkyl, carbamyl, keto, thioketo, thiol, alkylthio, sulfonyl, sulfonamido, thioalkoxy, aryl, nitro, and the like.

As used herein the term “alkylene” refers to a linear or branched radical having from about 1 to 10, 1 to 8, 1 to 6, or 2 to 6 carbon atoms and having attachment points for two or more covalent bonds. Examples of such radicals are methylene, ethylene, propylene, butylene, pentylene, hexylene, ethylidene, methylethylene, and isopropylidene. When an alkenylene radical is present as a substituent on another radical it is typically considered to be a single substituent rather than a radical formed by two substituents.

As used herein the term “alkenylene” refers to a linear or branched radical having from about 2 to 10, 2 to 8, or 2 to 6 carbon atoms, at least one double bond, and having attachment points for two or more covalent bonds. Examples of alkenylene radicals include 1,1-vinylidene (—CH₂═C—), 1,2-vinylidene (—CH═CH—), and 1,4-butadienyl (—CH═CH—CH═CH—).

As used herein the term “halo” refers to a halogen such as fluorine, chlorine, bromine or iodine atoms.

As used herein the term “hydroxyl” or “hydroxy” refers to an —OH group.

As used herein the term “cyano” refers to a carbon radical having three of four covalent bonds shared by a nitrogen atom, in particular —C≡N. A cyano group may be substituted with substituents described herein.

As used herein the term “alkoxy” refers to a linear or branched oxy-containing radical having an alkyl portion of one to about ten carbon atoms, such as a methoxy radical, which may be substituted. In aspects of the invention an alkoxy radical may comprise about 1-10, 1-8, 1-6, or 1-3 carbon atoms. In embodiments of the invention, an alkoxy radical comprises about 1-6 carbon atoms and includes a C₁-C₆ alkyl-O-radical wherein C₁-C₆ alkyl has the meaning set out herein. Examples of alkoxy radicals include without limitation methoxy, ethoxy, propoxy, butoxy, isopropoxy and tert-butoxy alkyls. An “alkoxy” radical may optionally be substituted with one or more substitutents disclosed herein including alkyl atoms to provide “alkylalkoxy” radicals; halo atoms, such as fluoro, chloro or bromo, to provide “haloalkoxy” radicals (e.g. fluoromethoxy, chloromethoxy, trifluoromethoxy, difluoromethoxy, trifluoroethoxy, fluoroethoxy, tetrafluoroethoxy, pentafluoroethoxy, and fluoropropox) and “haloalkoxyalkyl” radicals (e.g. fluoromethoxymethyl, chloromethoxyethyl, trifluoromethoxymethyl, difluoromethoxyethyl, and trifluoroethoxymethyl).

As used herein the term “alkenyloxy” refers to linear or branched oxy-containing radicals having an alkenyl portion of about 2 to 10 carbon atoms, such as an ethenyloxy or propenyloxy radical. An alkenyloxy radical may be a “lower alkenyloxy” radical having about 2 to 6 carbon atoms. Examples of alkenyloxy radicals include without limitation ethenyloxy, propenyloxy, butenyloxy, and isopropenyloxy alkyls. An “alkenyloxy” radical may be substituted with one or more substitutents disclosed herein including halo atoms, such as fluoro, chloro or bromo, to provide “haloalkenyloxy” radicals (e.g. trifluoroethenyloxy, fluoroethenyloxy, difluoroethenyloxy, and fluoropropenyloxy).

A “carbocylic” includes radicals derived from a saturated or unstaturated, substituted or unsubstituted 5 to 14, 5 to 12, or 5 to 10 member organic nucleus whose ring forming atoms (other than hydrogen) are solely carbon. Examples of carbocyclic radicals are cycloalkyl, cycloalkenyl, aryl, in particular phenyl, naphthyl, norbornanyl, bicycloheptadienyl, tolulyl, xylenyl, indenyl, stilbenyl, terphenylyl, diphenylethylenyl, phenylcyclohexyl, acenapththylenyl, anthracenyl, biphenyl, bibenzylyl, and related bibenzylyl homologs, octahydronaphthyl, tetrahydronaphthyl, octahydroquinolinyl, dimethoxytetrahydronaphthyl and the like.

As used herein, the term “cycloalkyl” refers to radicals having from about 3 to 15, 3 to 10, 3 to 8, or 3 to 6 carbon atoms and containing one, two, three, or four rings wherein such rings may be attached in a pendant manner or may be fused. In aspects of the invention, “cycloalkyl” refers to an optionally substituted, saturated hydrocarbon ring system containing 1 to 2 rings and 3 to 7 carbons per ring which may be further fused with an unsaturated C₃—C₇ carbocylic ring. Examples of cycloalkyl groups include single ring structures such as cyclopropyl, cyclobutyl, cyclopentyl, cyclohexyl, cycloheptyl, cyclooctyl, cyclononyl, cyclodecyl, cyclododecyl, and the like, or multiple ring structures such as adamantanyl, and the like. In certain aspects of the invention the cycloalkyl radicals are “lower cycloalkyl” radicals having from about 3 to 10, 3 to 8, 3 to 6, or 3 to 4 carbon atoms, in particular cyclopropyl, cyclobutyl, cyclopentyl, cyclohexyl and cycloheptyl. The term “cycloalkyl” also embraces radicals where cycloalkyl radicals are fused with aryl radicals or heterocyclyl radicals. A cycloalkyl radical may be optionally substituted with groups as disclosed herein.

In aspects of the invention, “substituted cycloalkyl” includes cycloalkyl groups having from 1 to 5 (in particular 1 to 3) substituents including without limitation alkyl, alkenyl, alkoxy, cycloalkyl, substituted cycloalkyl, acyl, acylamino, acyloxy, amino, aminoacyl, aminoacyloxy, oxyacylamino, cyano, halogen, hydroxyl, carboxyl, carboxylalkyl, keto, thioketo, thiol, thioalkoxy, aryl, aryloxy, heteroaryl, heteroaryloxy, hydroxyamino, alkoxyamino, and nitro.

As used herein in respect to certain aspects of the invention, the term “cycloaliphatic” refers to a cycloalkane possessing less than 8 carbons or a fused ring system consisting of no more than three fused cycloaliphatic rings. Examples of such groups include, but are not limited to, decalin and the like.

As used herein in respect to certain aspects of the invention, the term “substituted cycloaliphatic” refers to a cycloalkane possessing less than 8 carbons or a fused ring system consisting of no more than three fused rings, and where at least one of the aliphatic hydrogen atoms has been replaced by a halogen, a nitro, a thio, an amino, a hydroxy, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to, 1-chlorodecalyl and the like.

A used herein, the term “cycloalkenyl” refers to radicals comprising about 4 to 16, 2 to 15, 2 to 10, 2 to 8, 4 to 10, 3 to 8, 3 to 7, 3 to 6, or 4 to 6 carbon atoms, one or more carbon-carbon double bonds, and one, two, three, or four rings wherein such rings may be attached in a pendant manner or may be fused. In certain aspects of the invention the cycloalkenyl radicals are “lower cycloalkenyl” radicals having three to seven carbon atoms. Examples of cycloalkenyl radicals include without limitation cyclobutenyl, cyclopentenyl, cyclohexenyl and cycloheptenyl. A cycloalkenyl radical may be optionally substituted with groups as disclosed herein, in particular 1, 2, or 3 substituents which may be the same or different.

As used herein the term “cycloalkoxy” refers to cycloalkyl radicals (in particular, cycloalkyl radicals having 3 to 15, 3 to 8 or 3 to 6 carbon atoms) attached to an oxy radical. Examples of cycloalkoxy radicals include cyclohexoxy and cyclopentoxy. A cycloalkoxy radical may be optionally substituted with groups as disclosed herein.

As used herein, the term “aryl”, alone or in combination, refers to a carbocyclic aromatic system containing one, two or three rings wherein such rings may be attached together in a pendant manner or may be fused. In aspects of the invention an aryl radical comprises 4 to 24 carbon atoms, in particular 4 to 10, 4 to 8, or 4 to 6 carbon atoms. Illustrative “aryl” radicals includes without limitation aromatic radicals such as phenyl, benzyl, naphthyl, indenyl, benzocyclooctenyl, benzocycloheptenyl, pentalenyl, azulenyl, tetrahydronaphthyl, indanyl, biphenyl, acephthylenyl, fluorenyl, phenalenyl, phenanthrenyl, and anthracenyl. An aryl radical may be optionally subsitituted with groups as disclosed herein, in particular hydroxyl, alkyl (“arylalkyl”), carbonyl, carboxyl, thiol (“thioalkyl”), amino, and/or halo, in particular a substituted aryl includes without limitation arylamine and arylalkylamine.

As used herein in respect to certain aspects of the invention, the term “substituted aryl” includes an aromatic ring, or fused aromatic ring system consisting of no more than three fused rings at least one of which is aromatic, and where at least one of the hydrogen atoms on a ring carbon has been replaced by a halogen, an amino, a hydroxy, a nitro, a thio, an alkyl, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to, hydroxyphenyl, chlorophenyl and the like.

In aspects of the invention, an aryl radical may be optionally subsitituted with one to four substituents such as alkyl, substituted alkyl, alkenyl, substituted alkenyl, alkynyl, substituted alkynyl, aryl, substituted aryl, aralkyl, halo, trifluoromethoxy, trifluoromethyl, hydroxy, alkoxy, alkanoyl, alkanoyloxy, aryloxy, aralkyloxy, amino, alkylamino, arylamino, aralkylamino, dialkylamino, alkanoylamino, thiol, alkylthio, ureido, nitro, cyano, carboxy, carboxyalkyl, carbamyl, alkoxycarbonyl, alkylthiono, arylthiono, arylsulfonylamine, sulfonic acid, alkysulfonyl, sulfonamido, aryloxy and the like. A substituent may be further substituted by hydroxy, halo, alkyl, alkoxy, alkenyl, alkynyl, aryl or aralkyl. In aspects of the invention an aryl radical is substituted with hydroxyl, alkyl, carbonyl, carboxyl, thiol, amino, and/or halo. The term “aralkyl” refers to an aryl or a substituted aryl group bonded directly through an alkyl group, such as benzyl. Other particular examples of substituted aryl radicals include chlorobenyzl, and amino benzyl.

As used herein, the term “aryloxy” refers to aryl radicals, as defined above, attached to an oxygen atom. Exemplary aryloxy groups include napthyloxy, quinolyloxy, isoquinolizinyloxy, and the like.

As used herein the term “arylalkoxy,” refers to an aryl group attached to an alkoxy group. Representative examples of arylalkoxy groups include, but are not limited to, 2-phenylethoxy, 3-naphth-2-ylpropoxy, and 5-phenylpentyloxy.

As used herein, the term “aroyl” refers to aryl radicals, as defined above, attached to a carbonyl radical as defined herein, including without limitation benzoyl and toluoyl. An aroyl radical may be optionally substituted with groups as disclosed herein.

As used herein the term “heteroaryl” refers to fully unsaturated heteroatom-containing ring-shaped aromatic radicals having at least one heteroatom selected from carbon, nitrogen, sulfur and oxygen. A heteroaryl radical may contain one, two or three rings and the rings may be attached in a pendant manner or may be fused. In aspects of the invention the term refers to fully unsaturated heteroatom-containing ring-shaped aromatic radicals having from 3 to 15, 3 to 10, 3 to 8, 5 to 15, 5 to 10, or 5 to 8 ring members selected from carbon, nitrogen, sulfur and oxygen, wherein at least one ring atom is a heteroatom. Examples of “heteroaryl” radicals, include without limitation, an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like; an unsaturated condensed heterocyclic group containing 1 to 5 nitrogen atoms, in particular, indolyl, isoindolyl, indolizinyl, indazolyl, quinazolinyl, pteridinyl, quinolizidinyl, phthalazinyl, naphthyridinyl, quinoxalinyl, cinnolinyl, phenanthridinyl, acridinyl, phenanthrolinyl, phenazinyl, carbazolyl, purinyl, benzimidazolyl, quinolyl, isoquinolyl, quinolinyl, isoquinolinyl, indazolyl, benzotriazolyl, tetrazolopyridazinyl and the like; an unsaturated 3 to 6-membered heteromonocyclic group containing an oxygen atom, in particular, 2-furyl, 3-furyl, pyranyl, and the like; an unsaturated 5 to 6-membered heteromonocyclic group containing a sulfur atom, in particular, thienyl, 2-thienyl, 3-thienyl, and the like; unsaturated 5 to 6-membered heteromonocyclic group containing 1 to 2 oxygen atoms and 1 to 3 nitrogen atoms, in particular, furazanyl, benzofurazanyl, oxazolyl, isoxazolyl, and oxadiazolyl; an unsaturated condensed heterocyclic group containing 1 to 2 oxygen atoms and 1 to 3 nitrogen atoms, in particular benzoxazolyl, benzoxadiazolyl and the like; an unsaturated 5 to 6-membered heteromonocyclic group containing 1 to 2 sulfur atoms and 1 to 3 nitrogen atoms, for example, thiazolyl, isothiazolyl, thiadiazolyl and the like; an unsaturated condensed heterocyclic group containing 1 to 2 sulfur atoms and 1 to 3 nitrogen atoms such as benzothiazolyl, benzothiadiazolyl and the like. The term also includes radicals where heterocyclic radicals are fused with aryl radicals, in particular bicyclic radicals such as benzofuranyl, benzothiophenyl, phthalazinyl, chromenyl, xanthenyl, and the like. A heteroaryl radical may be optionally substituted with groups as disclosed herein, for example with an alkyl, amino, halogen, etc., in particular a heteroarylamine.

In aspects of the invention, the term refers to an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, tetrazolyl and the like.

A heteroaryl radical may be optionally substituted with groups disclosed herein, for example with an alkyl, amino, halogen, etc., in particular a substituted heteroaryl radical is a heteroarylamine.

The term “heterocyclic” refers to saturated and partially saturated heteroatom-containing ring-shaped radicals having at least one heteroatom selected from carbon, nitrogen, sulfur and oxygen. A heterocylic radical may contain one, two or three rings wherein such rings may be attached in a pendant manner or may be fused. In an aspect, the term refers to a saturated and partially saturated heteroatom-containing ring-shaped radicals having from about 3 to 15, 3 to 10, 5 to 15, 5 to 10, or 3 to 8 ring members selected from carbon, nitrogen, sulfur and oxygen, wherein at least one ring atom is a heteroatom. Examplary saturated heterocyclic radicals include without limitation a saturated 3 to 6-membered heteromonocylic group containing 1 to 4 nitrogen atoms [e.g. pyrrolidinyl, imidazolidinyl, piperidinyl, and piperazinyl]; a saturated 3 to 6-membered heteromonocyclic group containing 1 to 2 oxygen atoms and 1 to 3 nitrogen atoms [e.g. morpholinyl; sydnonyl]; and, a saturated 3 to 6-membered heteromonocyclic group containing 1 to 2 sulfur atoms and 1 to 3 nitrogen atoms [e.g., thiazolidinyl] etc. Examples of partially saturated heterocyclyl radicals include without limitation dihydrothiophene, dihydropyranyl, dihydrofuranyl and dihydrothiazolyl. Illustrative heterocyclic radicals include without limitation aziridinyl, azetidinyl, 2-pyrrolinyl, 3-pyrrolinyl, pyrrolidinyl, azepinyl, 1,3-dioxolanyl, 2H-pyranyl, 4H-pyranyl, piperidinyl, 1,4-dioxanyl, morpholinyl, pyrazolinyl, 1,4-dithianyl, thiomorpholinyl, 1,2,3,6-tetrahydropyridinyl, oxiranyl, oxetanyl, tetrahydrofuranyl, tetrahydropyranyl, tetrahydropyridinyl, tetrahydrothiopyranyl, thioxanyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3H-indolyl, quinuclidinyl, quinolizinyl, and the like.

As used herein in respect to certain aspects of the invention, the term “heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur. Examples of such groups include, but are not limited to, morpholino and the like.

As used herein in respect to certain aspects of the invention, the term “substituted heterocyclic” refers to a cycloalkane and/or an aryl ring system, possessing less than 8 carbons, or a fused ring system consisting of no more than three fused rings, where at least one of the ring carbon atoms is replaced by oxygen, nitrogen or sulfur, and where at least one of the aliphatic hydrogen atoms has been replaced by a halogen, hydroxy, a thio, nitro, an amino, a ketone, an aldehyde, an ester, an amide, a lower aliphatic, a substituted lower aliphatic, or a ring (aryl, substituted aryl, cycloaliphatic, or substituted cycloaliphatic). Examples of such groups include, but are not limited to 2-chloropyranyl.

The foregoing heteroaryl and heterocyclic groups may be C-attached or N-attached (where such is possible).

As used herein the term “sulfonyl”, used alone or linked to other terms such as alkylsulfonyl or arylsulfonyl, refers to the divalent radicals —SO₂—. In aspects of the invention, the sulfonyl group may be attached to a substituted or unsubstituted hydroxyl, alkyl group, ether group, alkenyl group, alkynyl group, aryl group, cycloalkyl group, cycloalkenyl group, cycloalkynyl group, heterocyclic group, carbohydrate, peptide, or peptide derivative.

The term “sulfinyl”, used alone or linked to other terms such as alkylsulfinyl (i.e. —S(O)-alkyl) or arylsulfinyl, refers to the divalent radicals —S(O)—.

The term “sulfonate” is art recognized and includes a group represented by the formula:

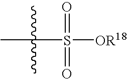

wherein R¹⁸ is an electron pair, hydrogen, alkyl, cycloalkyl, aryl, alkenyl, alkynyl, cycloalkenyl, cycloalkynyl, heterocyclic, carbohydrate, peptide, or peptide derivative.

The term “sulfate”, used alone or linked to other terms, is art recognized and includes a group that can be represented by the formula:

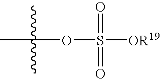

wherein R¹⁹ is an electron pair, hydrogen, alkyl, alkenyl, alkynyl, cycloalkyl, cycloalkenyl, cycloalkynyl, aryl, heterocyclic, carbohydrate, peptide or peptide derivative.

The term “sulfoxide” refers to the radical —S═O.

As used herein the term “amino”, alone or in combination, refers to a radical where a nitrogen atom (N) is bonded to three substituents being any combination of hydrogen, hydroxyl, alkyl, cycloalkyl, alkenyl, alkynyl, aryl, silyl, heterocyclic, or heteroaryl with the general chemical formula —NR³⁸R³⁹ where R³⁸ and R³⁹ can be any combination of hydrogen, hydroxyl, alkyl, cycloalkyl, alkoxy, alkenyl, alkynyl, aryl, carbonyl carboxyl, amino, silyl, heteroaryl, or heterocyclic which may or may not be substituted. Optionally one substituent on the nitrogen atom may be a hydroxyl group (—OH) to provide an amine known as a hydroxylamine. Illustrative examples of amino groups are amino (—NH₂), alkylamino, acylamino, cycloamino, acycloalkylamino, arylamino, arylalkylamino, and lower alkylsilylamino, in particular methylamino, ethylamino, dimethylamino, 2-propylamino, butylamino, isobutylamino, cyclopropylamino, benzylamino, allylamino, hydroxylamino, cyclohexylamino, piperidinyl, hydrazinyl, benzylamino, diphenylmethylamino, tritylamino, trimethylsilylamino, and dimethyl-tert.-butylsilylamino, which may or may not be substituted.

As used herein the term “thiol” means —SH. A thiol may be substituted with a substituent disclosed herein, in particular alkyl(thioalkyl), aryl(thioaryl), alkoxy(thioalkoxy) or carboxyl.

The term “sulfenyl” used alone or linked to other terms such as alkylsulfenyl, refers to the radical —SR²⁵ wherein R²⁵ is not hydrogen. In aspects of the invention R²⁵ is substituted or unsubstituted alkyl, cycloalkyl, alkenyl, alkynyl, aryl, silyl, silylalkyl, heterocyclic, heteroaryl, carbonyl, carbamoyl, alkoxy, or carboxyl.

As used herein, the term “thioalkyl”, alone or in combination, refers to a chemical functional group where a sulfur atom (S) is bonded to an alkyl, which may be substituted. Examples of thioalkyl groups are thiomethyl, thioethyl, and thiopropyl. A thioalkyl may be substituted with a substituted or unsubstitute carboxyl, aryl, heterocylic, carbonyl, or heterocyclic.

As used herein the term “thioaryl”, alone or in combination, refers to a chemical functional group where a sulfur atom (S) is bonded to an aryl group with the general chemical formula —SR²⁶ where R²⁶ is aryl which may be substituted. Illustrative examples of thioaryl groups and substituted thioaryl groups are thiophenyl, chlorothiophenyl, para-chlorothiophenyl, thiobenzyl, 4-methoxy-thiophenyl, 4-nitro-thiophenyl, and para-nitrothiobenzyl.

As used herein the term “thioalkoxy”, alone or in combination, refers to a chemical functional group where a sulfur atom (S) is bonded to an alkoxy group with the general chemical formula —SR²⁷ where R²⁷ is an alkoxy group which may be substituted. A “thioalkoxy group” may have 1-6 carbon atoms i.e. a —S—(O)—C₁-C₆ alkyl group wherein C₁-C₆ alkyl have the meaning as defined above. Illustrative examples of a straight or branched thioalkoxy group or radical having from 1 to 6 carbon atoms, also known as a C₁-C₆ thioalkoxy, include thiomethoxy and thioethoxy.

A thiol may be substituted with a substituted or unsubstituted heteroaryl or heterocyclic, in particular a substituted or unsubstituted saturated 3 to 6-membered heteromonocylic group containing 1 to 4 nitrogen atoms [e.g. pyrrolidinyl, imidazolidinyl, piperidinyl, and piperazinyl] or a saturated 3 to 6-membered heteromonocyclic group containing 1 to 2 oxygen atoms and 1 to 3 nitrogen atoms [e.g. morpholinyl; sydnonyl], especially a substituted morpholinyl or piperidinyl.

As used herein, the term “carbonyl” refers to a carbon radical having two of the four covalent bonds shared with an oxygen atom.

As used herein, the term “carboxyl”, alone or in combination, refers to —C(O)OR¹⁴— or —C(═O)OR¹⁴ wherein R¹⁴ is hydrogen, alkyl, alkenyl, alkynyl, cycloalkyl, cycloalkenyl, amino, thiol, aryl, heteroaryl, thioalkyl, thioaryl, thioalkoxy, a heteroaryl, or a heterocyclic, which may optionally be substituted. In particular aspects of the invention, —C(O)OR¹⁴ provides an ester or an amino acid derivative. An esterified form is also particularly referred to herein as a “carboxylic ester”. In aspects of the invention a “carboxyl” may be substituted, in particular substituted with alkyl which is optionally substituted with one or more of amino, amino, halo, alkylamino, aryl, carboxyl or a heterocyclic. Examples of carboxyl groups are methoxycarbonyl, butoxycarbonyl, tertalkoxycarbonyl such as tert.butoxycarbonyl, arylmethyoxycarbonyl having one or two aryl radicals including without limitation phenyl optionally substituted by for example lower alkyl, lower alkoxy, hydroxyl, halo, and/or nitro, such as benzyloxycarbonyl, methoxybenxyloxycarbonyl, diphenylmethoxycarbonyl, 2-bromoethoxycarbonyl, 2-iodoethoxycarbonyltert.butylcarbonyl, 4-nitrobenzyloxycarbonyl, diphenylmethoxycarbonyl, benzhydroxycarbonyl, di-(4-methoxyphenyl-methoxycarbonyl, 2-bromoethoxycarbonyl, 2-iodoethoxycarbonyl, 2-trimethylsilylethoxycarbonyl, or 2-triphenylsilylethoxycarbonyl. Additional carboxyl groups in esterified form are silyloxycarbonyl groups including organic silyloxycarbonyl. The silicon substituent in such compounds may be substituted with lower alkyl (e.g. methyl), alkoxy (e.g. methoxy), and/or halo (e.g. chlorine). Examples of silicon substituents include trimethylsilyl and dimethyltert.butylsilyl. In aspects of the invention, the carboxyl group may be an alkoxy carbonyl, in particular methoxy carbonyl, ethoxy carbonyl, isopropoxy carbonyl, t-butoxycarbonyl, t-pentyloxycarbonyl, or heptyloxy carbonyl, especially methoxy carbonyl or ethoxy carbonyl.

As used herein, the term “carbamoyl”, alone or in combination, refers to amino, monoalkylamino, dialkylamino, monocycloalkylamino, alkylcycloalkylamino, and dicycloalkylamino radicals, attached to one of two unshared bonds in a carbonyl group.

As used herein, the term “carboxamide” refers to the group —CONH—.

As used herein, the term “nitro” means —NO₂—.

As used herein, the term “acyl”, alone or in combination, means a carbonyl or thiocarbonyl group bonded to a radical selected from, for example, optionally substituted, hydrido, alkyl (e.g. haloalkyl), alkenyl, alkynyl, alkoxy (“acyloxy” including acetyloxy, butyryloxy, iso-valeryloxy, phenylacetyloxy, benzoyloxy, p-methoxybenzoyloxy, and substituted acyloxy such as alkoxyalkyl and haloalkoxy), aryl, halo, heterocyclyl, heteroaryl, sulfinyl (e.g. alkylsulfinylalkyl), sulfonyl (e.g. alkylsulfonylalkyl), cycloalkyl, cycloalkenyl, thioalkyl, thioaryl, amino (e.g alkylamino or dialkylamino), and aralkoxy. Illustrative examples of “acyl” radicals are formyl, acetyl, 2-chloroacetyl, 2-bromacetyl, benzoyl, trifluoroacetyl, phthaloyl, malonyl, nicotinyl, and the like.

In aspects of the invention, “acyl” refers to a group —C(O)R⁶⁴, where R⁶⁴ is hydrogen, alkyl, cycloalkyl, cycloheteroalkyl, aryl, arylalkyl, heteroalkyl, heteroaryl, and heteroarylalkyl. Examples include, but are not limited to formyl, acetyl, cyclohexylcarbonyl, cyclohexylmethylcarbonyl, benzoyl, benzylcarbonyl and the like.

As used herein the term “phosphonate” refers to a C—PO(OH)₂ or C—PO(OR⁶⁵)₂ group wherein R⁶⁵ is alkyl or aryl which may be substituted.

As used herein, “ureido” refers to the group “—NHCONH—”. A ureido radical includes an alkylureido comprising a ureido substituted with an alkyl, in particular a lower alkyl attached to the terminal nitrogen of the ureido group. Examples of an alkylureido include without limitation N′-methylureido, N′-ethylureido, N′-n-propylureido, N′-i-propylureido and the like. A ureido radical also includes a N′,N′-dialkylureido group containing a radical —NHCON where the terminal nitrogen is attached to two optionally substituted radicals including alkyl, aryl, heterocylic, and heteroaryl.

The terms used herein for radicals including “alkyl”, “alkoxy”, “alkenyl”, “alkynyl”, “hydroxyl” etc. refer to both unsubstituted and substituted radicals. The term “substituted,” as used herein, means that any one or more moiety on a designated atom (e.g., hydrogen) is replaced with a selection from a group disclosed herein, provided that the designated atom's normal valency is not exceeded, and that the substitution results in a stable compound. Combinations of substituents and/or radicals are permissible only if such combinations result in stable compounds. “Stable compound” refers to a compound that is sufficiently robust to survive isolation to a useful degree of purity from a reaction mixture, and formulation into an efficacious therapeutic agent.

A radical in a pyridazine compound may be substituted with one or more substituents apparent to a person skilled in the art including without limitation alkyl, alkoxy, alkenyl, alkynyl, alkanoyl, alkylene, alkenylene, hydroxyalkyl, haloalkyl, haloalkylene, haloalkenyl, alkoxy, alkenyloxy, alkenyloxyalkyl, alkoxyalkyl, aryl, alkylaryl, haloalkoxy, haloalkenyloxy, heterocyclic, heteroaryl, alkylsulfonyl, sulfinyl, sulfonyl, sulfenyl, alkylsulfinyl, aralkyl, heteroaralkyl, cycloalkyl, cycloalkenyl, cycloalkoxy, cycloalkenyloxy, amino, oxy, halo, azido, thio, ═O, ═S, cyano, hydroxyl, phosphonato, phosphinato, thioalkyl, alkylamino, arylamino, arylsulfonyl, alkylcarbonyl, arylcarbonyl, heteroarylcarbonyl, heteroarylsulfinyl, heteroarylsulfony, heteroarylamino, heteroaryloxy, heteroaryloxylalkyl, arylacetamidoyl, aryloxy, aroyl, aralkanoyl, aralkoxy, aryloxyalkyl, haloaryloxyalkyl, heteroaroyl, heteroaralkanoyl, heteroaralkoxy, heteroaralkoxyalkyl, thioaryl, arylthioalkyl, alkoxyalkyl, and acyl groups. These substitutents may themselves be substituted.

A chemical substituent is “pendant” from a radical if it is bound to an atom of the radical. In this context, the substituent can be pending from a carbon atom of a radical, a carbon atom connected to a carbon atom of the radical by a chain extender, or a heteroatom of the radical. The term “fused” means that a second ring is present (i.e, attached or formed) by having two adjacent atoms in common or shared with the first ring.

Pyridazine compounds, in particular compounds of the Formula I, II, III, IV, or V can be prepared using reactions and methods generally known to the person of ordinary skill in the art, having regard to that knowledge and the disclosure of this application including the Examples. The reactions are performed in a solvent appropriate to the reagents and materials used and suitable for the reactions being effected. It will be understood by those skilled in the art of organic synthesis that the functionality present on the compounds should be consistent with the proposed reaction steps. This will sometimes require modification of the order of the synthetic steps or selection of one particular process scheme over another in order to obtain a desired compound of the invention. It will also be recognized that another major consideration in the development of a synthetic route is the selection of the protecting group used for protection of the reactive functional groups present in the compounds. An authoritative account describing the many alternatives to the skilled artisan is Greene and Wuts (Protective Groups In Organic Synthesis, Wiley and Sons, 1991).

The starting materials and reagents used in preparing the pyridazine compounds are either available from commercial suppliers or are prepared by methods well known to a person of ordinary skill in the art, following procedures described in such references as Fieser and Fieser's Reagents for Organic Synthesis, vols. 1-17, John Wiley and Sons, New York, N.Y., 1991; Rodd's Chemistry of Carbon Compounds, vols. 1-5 and supps., Elsevier Science Publishers, 1989; Organic Reactions, vols. 1-40, John Wiley and Sons, New York, N.Y., 1991; March J.: Advanced Organic Chemistry, 4th ed., John Wiley and Sons, New York, N.Y.; and Larock: Comprehensive Organic Transformations, VCH Publishers, New York, 1989.

The starting materials, intermediates, and pyridazine compounds may be isolated and purified using conventional techniques, such as precipitation, filtration, distillation, crystallization, chromatography, and the like. The pyridazine compounds may be characterized using conventional methods, including physical constants and spectroscopic methods, in particular HPLC.

Pyridazine compounds which are basic in nature can form a wide variety of different salts with various inorganic and organic acids. In practice is it desirable to first isolate a pyridazine compound from the reaction mixture as a pharmaceutically unacceptable salt and then convert the latter to the free base compound by treatment with an alkaline reagent and subsequently convert the free base to a pharmaceutically acceptable acid addition salt. The acid addition salts of the base compounds of the pyridazine compounds are readily prepared by treating the base compound with a substantially equivalent amount of the chosen mineral or organic acid in an aqueous solvent medium or in a suitable organic solvent such as methanol or ethanol. Upon careful evaporation of the solvent, the desired solid salt is obtained.

Pyridazine compounds which are acidic in nature are capable of forming base salts with various pharmacologically acceptable cations. These salts may be prepared by conventional techniques by treating the corresponding acidic compounds with an aqueous solution containing the desired pharmacologically acceptable cations and then evaporating the resulting solution to dryness, preferably under reduced pressure. Alternatively, they may be prepared by mixing lower alkanolic solutions of the acidic compounds and the desired alkali metal alkoxide together and then evaporating the resulting solution to dryness in the same manner as before. In either case, stoichiometric quantities of reagents are typically employed to ensure completeness of reaction and maximum product yields.

In particular aspects, a compound of the formula II wherein R¹¹ is hydrogen and R¹⁰ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, may be prepared by reacting a compound with a structure of formula II wherein R¹⁰ is halo, in particular chloro, and R¹¹ is hydrogen, with boronic acid substituted with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, under suitable conditions to prepare a compound of the formula II wherein R¹¹ is hydrogen and R¹⁰ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl. In an embodiment, R¹⁰ is phenyl substituted with halo.

In another aspect, a compound of the formula II wherein R¹¹ is hydrogen and R¹⁰ is a substituted aryl is prepared by reacting a compound with the structure of formula II wherein R¹⁰ is halo, in particular chloro, and R¹¹ is hydrogen, with a substituted aryl boronic acid under suitable conditions.

In another aspect, a compound of the formula II wherein R¹⁰ is hydrogen and R¹¹ is alkyl is prepared by reacting a compound with the structures of formula II wherein R¹¹ is halo, in particular chloro, and R¹⁰ is hydrogen, with an alkyl boronic acid under suitable conditions. In an embodiment, R¹¹ is lower alkyl, in particular methyl or ethyl, and a compound of the formula II wherein R¹¹ is chloro is reacted with lower alkyl boronic acid, in particular methyl or ethyl boronic acid under suitable conditions.

In another aspect, a compound of the formula II is prepared wherein R¹⁰ is hydrogen and R¹¹ is an alkyl by reacting a pyridazine substituted at the C3 position with halo (e.g., chloro), at the C4 position with alkyl, and at the 6 position with phenyl, with 2-(piperidin-4-yloxy)pyrimidine under suitable conditions to prepare a compound of the formula II wherein R¹⁰ is hydrogen and R¹¹ is an alkyl. In an embodiment, R¹¹ is methyl or ethyl.

In another aspect, a compound of the formula II wherein R¹⁰ is hydrogen and R¹¹ is aryl is prepared by reacting a compound with the structure of formula II wherein R¹⁰ is hydrogen and R¹¹ is halo (e.g., chloro), with pyridazine substituted at the C3 position with halo (e.g., chloro), at the C4 position with aryl, and at the 6 position with phenyl, with 2-(piperidin-4-yloxy)pyrimidine under suitable conditions. In an embodiment, R¹¹ is phenyl.

In another aspect, a compound of the formula II is prepared wherein R¹⁰ is hydrogen and R¹¹ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl by reacting a compound of the formula II wherein R¹¹ is halo, in particular chloro, and R¹⁰ is hydrogen, with a boronic acid substituted with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, under suitable conditions.

In an embodiment, a compound of the formula II is prepared wherein R¹⁰ is hydrogen and R¹¹ is pyridinyl by reacting a compound of the formula II wherein R¹¹ is halo, in particular chloro, and R¹⁰ is hydrogen, with a pyridinyl boronic acid under suitable conditions.

In another aspect, a compound of the formula II is prepared wherein R¹⁰ is hydrogen and R¹¹ is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl by reacting a pyridazine substituted at the C3 position with halo, at the C4 position with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, and at the 6 position with phenyl, with 2-(piperidin-4-yloxy)pyrimidine under suitable conditions.

In an embodiment, a compound of the formula II is prepared wherein R¹⁰ is hydrogen and R¹¹ is pyridinyl by reacting a pyridazine substituted at the C3 position with halo, at the C4 position with pyridinyl, and at the 6 position with phenyl, with 2-(piperidin-4-yloxy)pyrimidine under suitable conditions to prepare a compound of the formula II wherein R¹⁰ is hydrogen and R¹¹ is pyridinyl.

In another aspect, a compound of the formula II is prepared wherein R¹⁰ is hydrogen and R¹¹ is piperidinyl or substituted piperidinyl by reacting a compound of the formula II wherein R¹¹ is halo, in particular chloro, and R¹⁰ is hydrogen with piperazinyl or substituted piperazinyl under suitable conditions.

In another aspect, a compound of the formula I is prepared wherein R¹ is piperazinyl or piperazinyl substituted with alkyl, aryl, or cycloalkyl, R² is aryl, R³, R⁴, R⁵ and R⁶ are hydrogen and R⁷ is absent, by reacting a pyridazine substituted at the C3 position with halo and at the C4 position with aryl, with a piperazinyl or piperazinyl substituted with alkyl, aryl, or cycloalkyl under suitable conditions.

In another aspect, a compound of the formula I is prepared wherein R¹ is piperazinyl or piperazinyl substituted with alkyl, aryl, or cycloalkyl, R² is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, R³, R⁴, R⁵ and R⁶ are hydrogen and R⁷ is absent, by reacting a pyridazine substituted at the C3 position with halo and at the C4 position with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, with piperazinyl or piperazinyl substituted with alkyl, aryl, or cycloalkyl under suitable conditions.

In another aspect, a compound of the formula I is prepared wherein R¹ is substituted amino in particular amino substituted with substituted morpholinyl, in particular morpholinoethyl, R² is aryl or an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, in particular pyridinyl, R³, R⁴, R⁵ and R⁶ are hydrogen and R⁷ is absent, by reacting a pyridazine substituted at the C3 position with halo, at the C4 position with aryl or an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, more particularly pyridinyl, with substituted amino in particular amino substituted with substituted morpholinyl, in particular morpholinoethyl, under suitable conditions.

In another aspect, a compound of the formula V is prepared wherein R⁵⁰ is aryl, R⁵¹ is hydrogen, and R⁵² is alkyl by reacting a pyridazine substituted at position C₃ with halo, at position C₄ with aryl and at position 6 with alkyl, with 1-(2-pyrimidyl)piperazine under suitable conditions.

In another aspect, a compound of the formula I is prepared wherein R¹ is substituted amino, R² is an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, in particular pyridinyl, R³, R⁴, R⁵ and R⁶ are hydrogen and R⁷ is absent by reacting a pyridazine substituted at the C₃ position with halo, at the C₄ position with an unsaturated 5 to 6 membered heteromonocyclyl group containing 1 to 4 nitrogen atoms, in particular, pyrrolyl, pyrrolinyl, imidazolyl, pyrazolyl, 2-pyridyl, 3-pyridyl, 4-pyridyl, pyridinyl, pyrimidinyl, pyrazinyl, pyridazinyl, triazolyl, or tetrazolyl, in particular pyridinyl, and at the C₆ position with phenyl, and a substituted amino under suitable conditions.

In the preparation of compounds of the Formula II, a precursor (see, for example, FIG. 10, compound (15)) that may be utilized can be obtained commercially and used directly for the synthesis of the illustrated compound MW01-3-183WH without further purification. Compounds may be synthesized with yields of 81-96%. All purified compounds may be characterized by HPLC, mass spectrometry and NMR in order to confirm syntheses. In FIG. 10, a synthetic scheme is shown, for synthesis of MW01-3-183WH.

Thus, in an aspect, a compound of the Formula II is prepared wherein a substituted 6-phenylpyridazine is reacted with 2-(piperazin-1yl)pyridmidine to produce a compound of the Formula II wherein R¹⁰ and R¹¹ are hydrogen. A compound of the formula II wherein R¹⁰ and R¹¹ are hydrogen can be reacted under suitable conditions and with suitable reagents to introduce the radicals R¹⁰ and R¹¹ which are independently hydrogen, hydroxyl, alkyl, alkenyl, alkynyl, alkylene, alkenylene, alkoxy, alkenyloxy, cycloalkyl, cycloalkenyl, aryl, aryloxy, arylalkoxy, aroyl, heteroaryl, heterocyclic, acyl, acyloxy, sulfonyl, sulfinyl, sulfenyl, amino, imino, azido, thiol, thioalkyl, thioalkoxy, thioaryl, nitro, ureido, cyano, halo, silyl, silyloxy, silylalkyl, silylthio, ═O, ═S, carboxyl, carbonyl, carbamoyl, or carboxamide.

The term “disorder” refers to disorders involving disruptions to normal neural electrical activity and/or function including without limitation conduction disturbances of the central nervous system (CNS), more particularly Seizure-Related Disorders. A disorder may be either or both of an acute or chronic nature of unknown origin, hereditary origin or it may be secondary to, for example, manipulation of the brain such as by surgery or radiation; alcohol, benzodiazepine, barbiturates or other drugs or chemical withdrawal; exposure to epileptogenic drugs; injury or trauma, stroke, cerebrovascular accident, fever, CNS inflammation or infection (e.g. meningitis), metabolic disturbance, or electroconvulsive therapy. The term also includes the emotional, cognitive, and motor symptoms resulting from these disorders.

The term “Seizure-Related Disorders” refers to a disorder characterized by conduction disturbances, electroconvulsions and/or seizures, in particular recurrent and/or excessive seizures. A “seizure” is a sudden disruption of the brain's normal electrical activity accompanied by altered consciousness and/or other neurological and behavioral manifestations. A seizure may be partial or generalized. In some aspects of the invention, a Seizure-Related Disorder includes Epilepsy, Ictogenesis, epileptogenesis, non-epileptic convulsions, eclampsia and convulsions due to administration of a convulsive agent or trauma to a subject.

Ictogenesis refers to the rapid and definitive electrical/chemical event which occurs over seconds or minutes. Epileptogenesis refers to a slow biochemical or histological process occurring over months to years involving transformation of the normal brain to a state susceptible to spontaneous, episodic, time-limited, recurrent seizures. Epileptogenesis involves a first initiation phase which occurs prior to the first seizure and often results from stroke, disease, trauma (e.g. caused by head injury or surgery). In the second phase of epileptogenesis, the brain, which is already susceptible to seizures, becomes more susceptible to more frequent and severe seizures.

The term “Epilepsy” refers to a chronic disorder of the brain characterized by transient but recurrent, excessive, or abnormal, disturbances to the electrical functions of the brain that may or may not associate with impairment or loss of consciousness and abnormal movements, sensation or behavior, in particular seizures. The term encompasses epileptic syndromes that are characterized by specific symptoms that include epileptic seizures. Such syndromes include but are not limited to, absence epilepsy, psychomotor epilepsy, temporal lobe epilepsy, frontal lobe epilepsy, occipital lobe epilepsy, parietal lobe epilepsy, Lennox-Gastaut syndrome, Rasmussen's encephalitis, childhood absence epilepsy, Ramsay Hunt Syndrome type II, benign epilepsy syndrome, benign infantile encephalopathy, benign neonatal convulsions, early myoclonic encephalopathy, progressive epilepsy and infantile epilepsy, mesial temporal lobe epilepsy, benign myoclonic epilepsy in infants, juvenile myoclonic epilepsy, juvenile absence epilepsy, epilepsy with generalized tonic clonic seizures in childhood, infantile spasms (West syndrome), epilepsy with continuous spike and waves in slow wave sleep (ESES), Laudau Kleffner syndrome, and Rasmussen's syndrome. A subject may suffer from any combination of these syndromes. In aspects of the invention, a subject suffers from partial seizures. In other aspects of the invention, a subject suffers from generalized seizures.

In aspects of the invention, a disorder is epileptogenesis. In other aspects of the invention, the disorder is epilepsy. In other aspects of the invention, the disorder is early-life seizures or pediatric epilepsy.

### Compositions and Kits

One or more pyridazine compound, in particular a compound of the Formula I, II, III, IV, or V, may be formulated into a pharmaceutical composition for administration to a subject. Therefore, the invention provides formulations including without limitation pills, tablets, caplets, soft and hard gelatin capsules, lozenges, sachets, cachets, vegicaps, liquid drops, elixirs, suspensions, emulsions, solutions, syrups, aerosols (as a solid or in a liquid medium) suppositories, sterile injectable solutions, and/or sterile packaged powders, which contain a pyridazine compound in particular a pure or substantially pure pyridazine compound.

Pharmaceutical compositions of the present invention or fractions thereof comprise suitable pharmaceutically acceptable carriers, excipients, and vehicles selected based on the intended form of administration, and consistent with conventional pharmaceutical practices. Particular compositions of the invention may contain a pyridazine compound that is pure or substantially pure. Suitable pharmaceutical carriers, excipients, and vehicles are described in the standard text, Remington: The Science and Practice of Pharmacy (21st Edition. 2005, University of the Sciences in Philadelphia (Editor), Mack Publishing Company), and in The United States Pharmacopeia: The National Formulary (USP 24 NF19) published in 1999.

A composition of the invention may include at least one buffering agent or solution. Suitable buffering agents include, but are not limited to hydrochloric, hydrobromic, hydroiodic, sulfuric, phosphoric, formic, acetic, propionic, succinic, glycolic, glucoronic, maleic, furoic, citric, glutamic, benzoic, anthranilic, salicylic, phenylacetic, mandelic, embonic, pamoic, methanesulfonic, ethanesulfonic, pantothenic, benzenesulfonic, stearic, sulfanilic, algenic, galacturonic acid and mixtures thereof. Additional agents that may be included are one or more of pregelatinized maize starch, polyvinyl pyrrolidone, hydroxypropyl methylcellulose, lactose, microcrystalline cellulose, calcium hydrogen phosphate, magnesium stearate, talc, silica, potato starch, sodium starch glycolate, sodium lauryl sulfate, sorbitol syrup, cellulose derivatives, hydrogenated edible fats, lecithin, acacia, almond oil, oily esters, ethyl alcohol, fractionated vegetable oils, methyl, propyl-p-hydroxybenzoates, sorbic acid and mixtures thereof. Buffering agents may additionally comprise one or more of dichlorodifluoromethane, trichloro fluoromethane, dichlorotetra fluoroethane, carbon dioxide, poly (N-vinyl pyrrolidone), poly (methylmethacrylate), polyactide, polyglycolide and mixtures thereof. In some aspects, a buffering agent may be formulated as at least one medium including without limitation a suspension, solution, or emulsion. In other aspects, a buffering agent may additionally comprise a formulatory agent including without limitation a pharmaceutically acceptable carrier, excipient, suspending agent, stabilizing agent or dispersing agent.

In aspects of the invention, a pharmaceutical composition is provided for oral administration of one or more pyridazine compounds for treatment of a disorder. By way of example for oral administration in the form of a capsule or tablet, the active component can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug component may be combined with any oral, non-toxic, pharmaceutically acceptable inert carrier such as ethanol, glycerol, water, and the like. Suitable binders (e.g. gelatin, starch, corn sweeteners, natural sugars including glucose; natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions. Compositions as described herein can further comprise wetting or emulsifying agents, or pH buffering agents.

In aspects of the invention, a composition of the invention is a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

Formulations for parenteral administration may include aqueous solutions, syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone.

Compositions for parenteral administration may include sterile aqueous or non-aqueous solvents, such as water, isotonic saline, isotonic glucose solution, buffer solution, or other solvents conveniently used for parenteral administration of therapeutically active agents. A composition intended for parenteral administration may also include conventional additives such as stabilizers, buffers, or preservatives, e.g. antioxidants such as methylhydroxybenzoate or similar additives.

Compositions of the invention can be formulated as pharmaceutically acceptable salts as described herein.

A compound of the formula I, II, III, IV or V or a composition of the invention may be sterilized by, for example, filtration through a bacteria retaining filter, addition of sterilizing agents to the compounds or composition, irradiation of the compounds or composition, or heating the compounds or composition. Alternatively, the compounds or compositions of the present invention may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the invention, such labeling would include amount, frequency, and method of administration.

According to the invention, a kit is provided. In an aspect, the kit comprises a compound of the formula I, II, III, IV or V or a formulation of the invention in kit form. The kit can be a package which houses a container which contains compounds of the formula I, II, III, IV or V or formulations of the invention and also houses instructions for administering the compounds or formulations to a subject. The invention further relates to a commercial package comprising compounds of the formula I, II, III, IV or V or formulations of the invention together with instructions for their use. In particular a label may include amount, frequency, and method of administration.

The invention also provides a pharmaceutical pack or kit comprising one or more containers filled with one or more of the ingredients of a composition of the invention to provide a beneficial effect including a therapeutic effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The invention also relates to articles of manufacture and kits containing materials useful for treating a disorder disclosed herein. An article of manufacture may comprise a container with a label. Examples of suitable containers include bottles, vials, and test tubes which may be formed from a variety of materials including glass and plastic. A container holds compounds of the formula I, II, III, IV or V or formulations of the invention which are effective for treating a disorder disclosed herein. The label on the container indicates that the compounds of the formula I, II, III, IV or V or formulations of the invention are used for treating a disorder disclosed herein and may also indicate directions for use. In aspects of the invention, a medicament or formulation in a container may comprise any of the medicaments or formulations disclosed herein.

In aspects of the invention, a kit of the invention comprises a container described herein. In particular aspects, a kit of the invention comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disorder disclosed herein). A medicament or formulation in a kit of the invention may comprise any of the formulations or compositions disclosed herein.

In aspects of the invention, the kits may be useful for any of the methods disclosed herein, including, without limitation treating a subject suffering from Epilepsy. Kits of the invention may contain instructions for practicing any of the methods described herein.

### Administration

A pyridazine compound and composition of the present invention can be administered by any means that produces contact of the active agent(s) with the agent's sites of action in the body of a subject or patient to produce a therapeutic effect, in particular a beneficial effect, in particular a sustained beneficial effect. A pyridazine compound or composition of the invention can be formulated for sustained release, for delivery locally or systemically. It lies within the capability of a skilled physician or veterinarian to select a form and route of administration that optimizes the effects of the compositions and treatments of the present invention to provide therapeutic effects, in particular beneficial effects, more particularly sustained beneficial effects.

Pyridazine compounds and compositions may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. Pyridazine compounds and compositions of the invention may be administered by intranasal route via topical use of suitable intranasal vehicles, or via a transdermal route, for example using conventional transdermal skin patches. A dosage protocol for administration using a transdermal delivery system may be continuous rather than intermittent throughout the dosage regimen. A sustained release formulation can also be used for the therapeutic agents.

In aspects of the invention the pyridazine compounds or compositions of the invention are administered by peripheral administration, in particular by intravenous administration, intraperitoneal administration, subcutaneous administration, intramuscular administration, oral administration, topical administration, transmucosal administration, or pulmonary administration.

A therapeutically effective dose of a pyridazine compound or composition of the invention for the treatment of a particular disorder or condition to provide effects, in particular beneficial effects, more particularly sustained beneficial effects, will depend on the nature of the disorder, and can be determined by standard clinical techniques. The precise dose to be employed in the formulation will also depend on the route of administration, and the seriousness of the disorder, and should be decided according to the judgment of the practitioner and each patient's circumstances.

Suitable dosage ranges for administration are particularly selected to provide therapeutic effects, in particular beneficial effects, more particularly sustained beneficial effects. A dosage range is generally effective for triggering the desired biological responses. The dosage ranges for a pyridazine compound are generally about 0.01 mg to about 3 g per kg, 0.01 mg to about 2 g per kg, 0.5 mg to about 2 g per kg, about 1 mg to about 1 g per kg, about 1mg to about 500 mg per kg, about 1 mg to about 200 mg per kg, about 1 mg to about 100 mg per kg, about 1 mg to about 50 mg per kg, about 10 mg to about 100 mg per kg, or about 30 mg to 70 mg per kg of the weight of a subject, once, twice, or more per day, preferably once daily.

In aspects of the invention the dosages ranges are about 0.01 to 3000 mg/kg, 0.01 to 2000 mg/kg, 0.5 to 2000 mg/kg, about 0.5 to 1000 mg/kg, 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 50 mg/kg, 0.1 to 20 mg/kg, 0.1 to 10 mg/kg, 0.1 to 6 mg/kg, 0.1 to 5 mg/kg, 0.1 to 3 mg/kg, 0.1 to 2 mg/kg, 0.1 to 1 mg/kg, 1 to 1000 mg/kg, 1 to 500 mg/kg, 1 to 400 mg/kg, 1 to 300 mg/kg, 1 to 200 mg/kg, 1 to 100 mg/kg, 1 to 50mg/kg, 1 to 20 mg/kg, 1 to 10 mg/kg, 1 to 6 mg/kg, 1 to 5 mg/kg, or 1 to 3 mg/kg, or 1 to 2.5 mg/kg, or less than or about 10 mg/kg, 5 mg/kg, 2.5 mg/kg, 1 mg/kg, or 0.5 mg/kg twice daily or less.

In embodiments of the invention, the dosages ranges are about 0.1 to 1000 mg/kg, 0.1 to 500 mg/kg, 0.1 to 400 mg/kg, 0.1 to 300 mg/kg, 0.1 to 200 mg/kg, 0.1 to 100 mg/kg, 0.1 to 75 mg/kg, 0.1 to 50 mg/kg, 0.1 to 25 mg/kg, 0.1 to 20 mg/kg, 0.1 to 15 mg/kg, 0.1 to 10 mg/kg, 0.1 to 9 mg/kg, 0.1 to 8 mg/kg, 0.1 to 7 mg/kg, 0.1 to 6 mg/kg, 0.1 to 5 mg/kg, 0.1 to 4 mg/kg, 0.1 to 3 mg/kg, 0.1 to 2 mg/kg, or 0.1 to 1 mg/kg.

A composition or treatment of the invention may comprise a unit dosage of a pyridazine compound to provide beneficial effects, in particular one or more of the beneficial effects set out herein. A “unit dosage” or “dosage unit” refers to a unitary i.e., a single dose which is capable of being administered to a patient, and which may be readily handled and packed, remaining as a physically and chemically stable unit dose comprising the active agent as such or a mixture with one or more solid or liquid pharmaceutical excipients, carriers, or vehicles.

A pyridazine compound can be provided once daily, twice daily, in a single dosage unit or multiple dosage units (i.e., tablets or capsules) having about 50 to about 10000 mg, 50 to about 2000 mg, 70 to about 7000 mg, 70 to about 6000 mg, 70 to about 5500 mg, 70 to about 5000 mg, 70 to about 4500 mg, 70 to about 4000 mg, 70 to about 3500 mg, 70 to about 3000 mg, 150 to about 2500 mg, 150 to about 2000 mg, 200 to about 2500, 200 to about 2000 mg, 200 to about 1500 mg, 700 to about 1200 mg, 70 mg to 1000 mg, 70 mg to 500 mg, in particular 200 to 2000 mg, 70 to 1200 mg, or 1000 mg.

The dosage regimen of the invention will vary depending upon known factors such as the pharmacodynamic characteristics of the agents and their mode and route of administration; the species, age, sex, health, medical condition, and weight of the patient, the nature and extent of the symptoms, the kind of concurrent treatment, the frequency of treatment, the route of administration, the renal and hepatic function of the patient, and the desired effect.

Thus, a subject may be treated with a pyridazine compound or a composition of the invention on substantially any desired schedule. A pyridazine compound or composition of the invention may be administered one or more times per day, in particular 1 or 2 times per day, once per week, once a month, twice a month or continuously. However, a subject may be treated less frequently, such as every other day or once a week, or more frequently. A pyridazine compound or a composition of the invention may be administered to a subject for about or at least about 24 hours, 2 days, 3 days, 1 week, 2 weeks to 4 weeks, 2 weeks to 6 weeks, 2 weeks to 8 weeks, 2 weeks to 10 weeks, 2 weeks to 12 weeks, 2 weeks to 14 weeks, 2 weeks to 16 weeks, 2 weeks to 6 months, 2 weeks to 12 months, 2 weeks to 18 months, or 2 weeks to 24 months, periodically or continuously.

Pyridazine compounds, compositions and treatment methods described herein are indicated as therapeutic agents or methods either alone or in conjunction with other therapeutic agents or other forms of treatment. They may be combined or formulated with one or more therapies or agents used to treat a condition described herein. Compositions of the invention may be administered concurrently, separately, or sequentially with other therapeutic agents or therapies. Therefore, compounds of the formula I, II, III, IV or V may be co-administered with one or more additional therapeutic agents for treating disorders disclosed herein as well as agents that are used for the treatment of complications resulting from or associated with a disorder disclosed herein, or general medications that treat or prevent side effects.

In aspects, the additional therapeutic agents comprise phenobarbital, valproate, levetiracetam, tiagabine, N-methyl-D-aspartate, alpha-amino-3-hydroxy-5-methylisoxazole-4-propionic acid (AMPA) antagonists, and neuotrophins and their receptors.

The invention will be described in greater detail by way of a specific example. The following example is offered for illustrative purposes, and is not intended to limit the invention in any manner.

### Examples

### Summary

Early-life seizures increase vulnerability to subsequent neurologic insult. The hypothesis that early-life seizures increase susceptibility to later neurologic injury by causing chronic glial activation was tested. To determine the mechanisms by which glial activation may modulate neurologic injury, both acute changes in pro-inflammatory cytokines and long-term changes in astrocyte and microglial activation and astrocyte glutamate transporters in a ‘two-hit’ model of kainic acid (KA)-induced seizures were examined. Methods: Postnatal day (P) 15 male rats were administered KA or phosphate buffered saline (PBS). On P45 animals either received a second treatment of KA or PBS. On P55, control (PBS-PBS), early-life seizure (KA-PBS), adult seizure (PBS-KA), and ‘two-hit’ (KA-KA) groups were examined for astrocyte and microglial activation, alteration in glutamate transporters and expression of the glial protein, clusterin. Results: P15 seizures resulted in an acute increase in hippocampal levels of IL-10 and S100B, followed by behavioral impairment and long-term increases in GFAP and S100B. Animals in the ‘two-hit’ group showed greater microglial activation, neurologic injury and susceptibility to seizures compared to the adult seizure group. Glutamate transporters increased following seizures but did not differ between these two groups. Treatment with Minozac, a small molecule inhibitor of proinflammatory cytokine up-regulation, following early-life seizures prevented both the long-term increase in activated glia and the associated behavioral impairment. Conclusions: These data suggest that glial activation following early-life seizures results in increased susceptibility to seizures in adulthood, in part through priming microglia and enhanced microglial activation. Glial activation may be a novel therapeutic target in pediatric epilepsy.

The following materials and methods were used in the study described in this example.

### Materials and Methods:

### Animal Use

A total of 195 male Sprague-Dawley rats (Charles River Laboratories, Cambridge, Mass.) were used in these experiments. Animals were divided into experimental groups as summarized in Tables 6 and 7. Within each group, where multiple outcome measures were required, surviving animals were randomly selected for each endpoint. All procedures were approved by the Institutional Animal Care and Use Committee of Children's Memorial Research Center, Chicago, Ill.

### Kainic Acid-Mediated Induction of Seizures

On either postnatal day (P)15 or 45, rats were administered either KA (Ocean Produce International, Nova Scotia) or equivalent volume of vehicle, phosphate-buffered saline (PBS, pH 7.4) (2). The dose of KA on P 15 (5 mg/kg) and P45 (15 mg/kg) was based on the age-dependent difference in threshold for KA-induced seizures (22). All injections were intraperitoneal (ip).

### ‘Two-Hit’ Model of Kainic Acid-Induced Seizures

To determine the long-term effects of early-life seizures occurring on P15, animals were allowed to recover for 30 days. On P45, rats were administered KA as previously described for the ‘twohit’ model of KA-induced neurologic injury (2). Control animals were administered PBS, and all animals were allowed to recover for a further 10 days before sacrifice on P55. Four experimental groups, including controls (PBS-PBS), newborn seizures (KA-PBS), adult seizures (PBS-KA), and ‘two-hit’ animals (KA-KA) were studied. Rats were perfused transcardially with PBS and fixed with 4% paraformaldehyde (PFA) in 0.1 M PBS, pH 7.4. Brains were excised and post-fixed in 4% PFA overnight. Brains were paraffin-embedded and sectioned for immunohistochemistry. For each outcome measure, two axial sections representing the hippocampus (sections between Bregma −7.34 mm, Paxinos plate 99, and Bregma −5.60 mm, Paxinos plate 106) were selected (23).

### Determination of Seizure Onset and Grading Severity of Seizures

Following administration of KA or PBS, all animals were monitored continuously for three hours. Time of latency to onset of first seizure (defined as onset of forelimb clonus) and severity of seizures were quantified as previously described (2). Only animals with grade IV seizures were included in this study.

### Assessment of Neuronal Injury

Neuronal injury was measured on five μm paraffin-embedded sections using Fluoro-Jade B (FJB) (Chemicon International, Temecula, Calif.) (24,25). The nuclear dye 4, 6-diamidino-2-phenyllindole (DAPI, Sigma, St. Louis, Mo.) was used to counterstain and to identify cell nuclei. CA1, CA2, CA3, dentate gyrus (DG) and polymorph dentate gyrus (PoDG) of the hippocampus were photographed at 10x magnification, and images were converted to grayscale for quantification of FJB-positive cells. The percentage of positive cells in the hippocampal regions specified was measured by thresholding for lightly stained, or FJB-positive cells (Metamorph, Universal Imaging Corporation, Sunnyvale, Calif.). The total FJB-positive cells in the hippocampus as well as hippocampal region-specific staining were obtained for each sample.

### Brain Homogenate Preparation and Protein Concentration Determination

Hippocampal homogenates were prepared by sonication in protease inhibitor cocktail (1 μg leupeptin (Sigma, St. Louis, Mo.), 0.001 M 4-dithio-L-threitol (DTT, Sigma), 0.002 M sodium orthovanadate (Sigma) and 0.001 M phenylmethanesulfonyl fluorid (PMSF, FLUKA, Switzerland) in 1 ml PBS). Total protein concentration was measured using commercially available reagents (BCA, Pierce, Rockford, Ill.).

### Measurement of Pro-Inflammatory Cytokines

Levels of IL-1β, IL-6, and TNF-α, were measured in hippocampal homogenates by enzyme linked immunosorbent assay using commercially available plates and reagents (ELISA; MesoScale Discovery, MSD, Gaithersburg, Md.). S100B levels were measured as described (26). Samples were analyzed in duplicates and compared with known concentrations of protein. Plates were analyzed using the SECTOR Imager 2400 (MSD).

### Hippocampal-Linked Task Testing

The Y-maze test of spontaneous alternation was used to evaluate hippocampus-dependent spatial learning (26, 27). Testing began on P27 when animals were first able to decide between the left or right arm of the maze. Testing was performed by a blinded observer on alternate days until P55. Each animal started in the vertical arm of the Y-maze. If the animal selected a different arm on the second run in the maze, it was scored as alternating. The percent alternation over the duration of testing was calculated for each animal.

### Western Blot Analysis of Factor H and Clusterin Levels Following Kainate-Induced Seizures

Changes in FH and clusterin levels in the hippocampus 24 hr following KA-induced seizures were quantified by Western blot using commercially available antibodies (1:2000 goat polyclonal anti-FH antibody, Quidel Corporation, San Diego, Calif.; 1:1000 goat polyclonal anti clusterin antibody, Santa Cruz Biotech, Santa Cruz, Calif.). Proteins were separated by conventional methods using polyacrylamide electrophoresis gels. Following incubation in secondary antibody (1:1000 anti-goat IgG-HRP for FH; 1:2000 anti-goat IgG-HRP for clusterin), protein bands were visualized by electrochemiluminescent detection (ECL Western Blotting Detection Reagents, Amersham Biosciences, Piscataway, N.J.). Bands of the appropriate size were quantified by relative densitometry using digitized images and OpenLab software (Improvision line, Lexington, Mass.). Values for FH and clusterin were normalized to GAPDH and β-tubulin respectively, and the data expressed as a ratio of the two bands (1:4000 GAPDH, Ambion, Austin, Tex.; 1:1000 β-tubulin. The E7 anti-β-tubulin antibody developed by Michael Klymkowsky was obtained from the Developmental Studies Hybridoma Bank developed under the auspices of the NICHD and maintained by The University of Iowa, Department of Biological Sciences, Iowa City, Iowa 52242).

### Immunohistochemistry

Immunohistochemical detection of hippocampal markers of glial activation and astrocyte glutamate transporters was performed in five μm paraffin-embedded sections using Vectastain Elite ABC immunodetection kits and diaminobenzidine substrate (DAB) (Vector Laboratories, Burlingame Calif.). The following primary antibodies were used: GFAP (1:1500, mouse monoclonal, Sigma); S100B (1:1500, rabbit polyclonal, DAKO Cytomation, Carpinteria, Calif.); Ibal (1:400, goat polyclonal, Abcam, Cambridge, Mass.); FH (1:400, goat polyclonal, Quidel); β-clusterin (1:100, goat polyclonal, Santa Cruz); GLAST (1:5000 guinea pig polyclonal, Chemicon International, Temecula, Calif.) and GLT-1 (1:5000, guinea pig polyclonal, Chemicon). Control sections were incubated in normal serum or PBS in place of primary antibody. In order to determine the specificity of the antibodies, selected slides were incubated with preadsorbed IgG in place of primary antibody (Rabbit IgG for S100B; Mouse IgG for GFAP). For clusterin, GLAST and GLT-1, control peptides were used instead of the primary antibodies to determine specific immunoreactivity. Sections were incubated for one hour at 38° C. with the appropriate biotinylated secondary antibody at 1:400 dilution (Vector).

### Immunofluorescent Double-Labeling of Clusterin Positive Cells

Double-labeling of clusterin-immunoreactive cells was performed with either the astrocyte marker, GFAP (1:1000) or the neuronal marker, NeuN (1:50, Chemicon). After rehydration and incubation in normal horse serum (NHS) for 20 min, sections were incubated with either primary antibody for 1 hr at room temperature. After a 30 min incubation with biotinylated secondary antibody (anti-mouse IgG, 1:400) tissues were incubated with Fluorescent Avidin D for 45 min (Vector), then blocked with Avidin and Biotin solutions and 10% NHS. Tissues were then incubated with the second primary antibody (clusterin, 1:50 dilution) for 1 hr. Sections were incubated with biotinylated anti-goat IgG secondary antibody (1:400) for 20 min, followed by Texas Red Avidin for 45 min (Vector). After washing with PBS, coverslips were mounted with Vectashield mounting media with DAPI (Vector).

### Peroxidase Double-Labeling of Clusterin-Immunoreactive Cells

Double-labeling of clusterin-immunoreactive cells was also performed with the microglial marker, Ibal or with the neuronal marker, NeuN. Standard immunohistochemical methods were followed but VIP (Vector) instead of DAB was used as the substrate for visualization. Following development with VIP, tissues were blocked and then incubated with Ibal antibody (1:400 dilution) overnight at 4° C. After incubation with biotinylated anti-goat IgG for 1 hr, tissues were treated with ABC solution (Vector) and developed with SG substrate (Vector) before mounting. A similar approach was used for double labeling of clusterin with NeuN (1:50).

### Image Acquisition and Quantification of Immunoreactive Cells

Sections were examined under brightfield microscopy by two blinded observers (Nikon Eclipse E800). For GFAP, S100B, Ibal, FH, clusterin, GLAST and GLT-1 sections, the CAI, CA2, CA3, DG and PoDG were photographed at 10× magnification, and images were converted to grayscale for quantification of immunoreactive cells. The percentage of positive cells in the hippocampal regions specified was measured by thresholding for dark objects indicative of immunoreactive cells (Metamorph, Universal Imaging Corporation, Sunnyvale, Calif.). The total hippocampal immunoreactivity as well as hippocampal region-specific immunoreactivity was obtained for each sample. Changes in the morphology of immunoreactive cells were not assessed.

### Attenuation of Pro-Inflammatory Cytokine Increase Following Kainate Administration

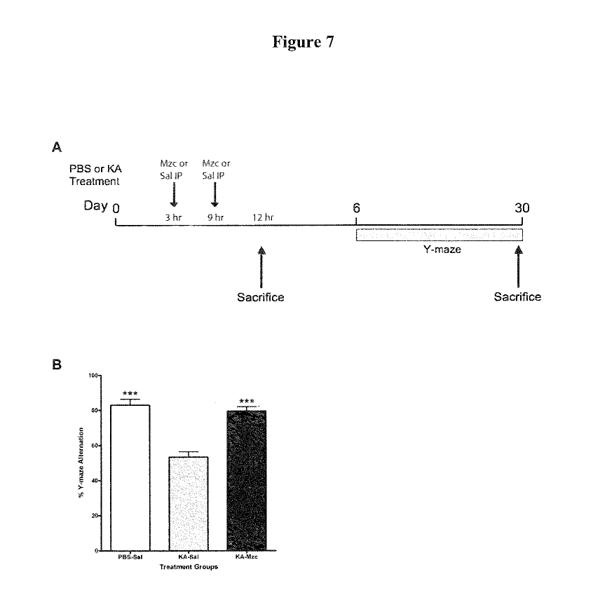

To determine whether suppression of early cytokine increases would prevent long-term glial activation and improve neurobehavioral outcome after early-life seizures, P15 rats were administered Minozac (Mzc). Mzc, 2-(4-(4-methyl-6-phenylpyridazin-3-yl)piperazin-1-yl)pyrimidine dihydrochloride monohydrate, is a bioavailable and CNS-penetrant, small molecule suppressor of brain pro-inflammatory cytokine up-regulation (19). Mzc was refined from a lead compound synthesized by chemical diversification of an inactive pyridazine fragment (26). Mzc (5 mg/kg) or diluent (saline) was administered via ip injection at 3 and 9 hr following KA injection on P15 (FIG. 7). Control animals were injected an equal volume of saline (Sal). At 12 hr, animals were sacrificed, and tissues were processed for analysis of pro-inflammatory cytokines by ELISA as above. A separate group of animals was allowed to recover for 30 d for Y-maze testing and immunohistochemical analysis of glial activation. Details of experimental groups and outcome measures are summarized in Table 7.

### Statistical Analysis

Values are expressed as mean±SEM for each group. Test for normality was performed for each data set. Student's t-test was used to compare two groups, and One-way analysis of variance (ANOVA) was performed to compare three or more groups. Tukey's Multiple Comparison Test was used for parametric measures and Dunn's post-test was applied for non-parametric data. Fisher's Exact Test was used to determine differences in seizure severity. Kaplan-Meier Survival Analysis and log rank test was performed to analyze differences in mortality between groups. Significance was defined as p<0.05 for all tests. Prism 4.0 (GraphPad Software, Inc., San Diego, Calif.) was used for statistical analyses.

### Results

### Comparison of Kainate-Induced Seizures on P15 and P45

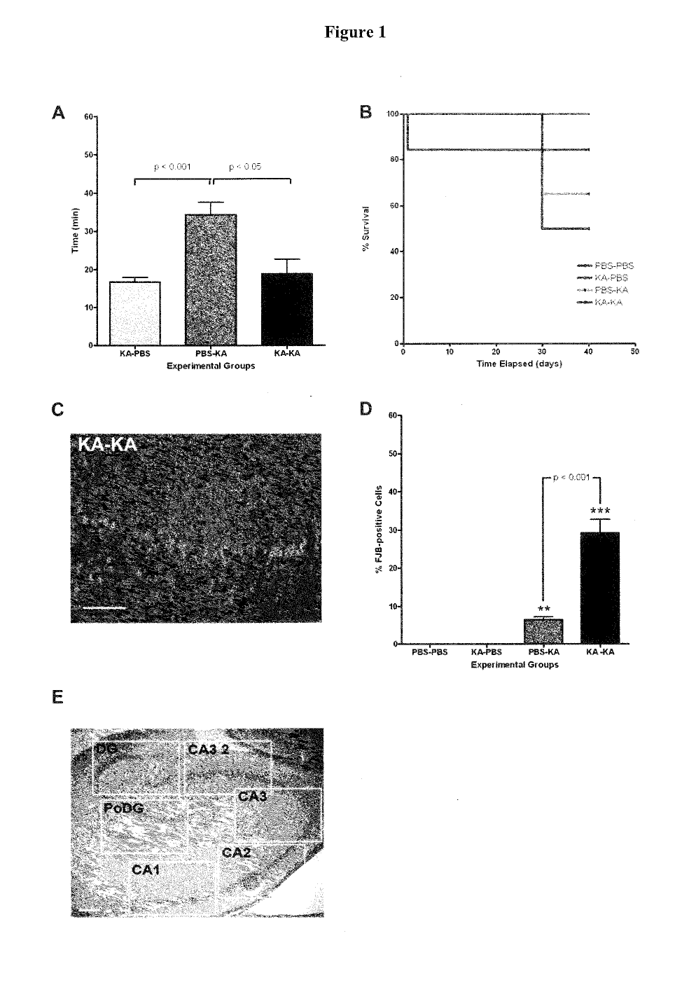

Seizure latency (data expressed as minutes±SEM; n) was significantly reduced in rats exposed to KA on P15 (P15 KA and KA-PBS; 16.7±1.2; n=30) compared to P45 (P45 KA and PBS-KA; 34.3±3.3; n=32) (FIG. 1A). There were no differences in severity of seizures between age groups. On P15, the majority of animals (P15 KA and KA-PBS; 27/31, 87%) experienced Grade IV seizures, which was not significantly different from the frequency of Grade IV seizures in the P45 treatment group (P45 KA and PBS-KA; 28/32, 88%). Survival in the immature rats exposed to KA (KA-P13S, 16/19, 84%) was not significantly different from the P45 group (PBS-KA, 13/20, 65%) (FIG. 1B).

### Early Life Seizures Increase Susceptibility to Seizures in Adulthood

Seizure susceptibility (FIG. 1) were first compared in the ‘two-hit’ group exposed to KA on both P15 and P45 (KA-KA) with rats first exposed to KA on P45 (PBS-KA). Seizure latency was significantly reduced in the ‘two-hit’ group (KA-KA, 18.8±3.8; n=12), compared to rats first administered KA on P45 (P45 KA and PBS-KA; 34.3±3.3; n=32) (FIG. 1A). Survival (FIG. 1B) was significantly decreased in the ‘two-hit’ group (6/12, 50%) compared to the PBS-KA animals (13/20, 65%). There were no significant inter-group differences in seizure severity. Grade IV seizures occurred in 88% (28/32) of animals exposed to KA on P45 (P45 KA and PBS-KA) and in 100% (12/12) of the ‘two-hit’ KA-KA animals. There were no significant differences in weight (g±SEM) between groups at the time of sacrifice on P55 (PBS-PBS, 316.7±6.1; KA-PBS, 327.7±8.4; PBS-KA, 292.6±9.0; KA-KA, 326.0±21.2).

Neuronal Injury Resulting from Seizures is Greater Following a ‘Second-Hit’

The degree of hippocampal neuronal injury (FIGS. 1C and D) in the adult seizure (PBS-KA) was compared to the ‘two-hit’ (KA-KA) groups using the fluorescent dye FJB (data expressed as % FJB-positive cells±SEM). A summary of the regions of interest for FJB and other immunohistochemical outcome measures is shown in FIG. 1E. No neuronal injury was detected either in controls (PBS-PBS; n=9) or the immature seizure group (KA-PBS; n=5). The ‘two-hit’ group had significantly greater neuronal injury (29.2±3.6; n=5) compared to littermates who first received KA as adults (PBS-KA, 6.4±0.8; n=7). The majority of FJB-positive cells in both groups were in the CA3 layer of the hippocampus. CAI, CA2 and DG also showed significant increases in labeled cells compared to controls. FJG-positive cells were also observed in the entorhinal and frontal cortex, lateral septal nuclei, piriform cortex, perirhinal cortex of these groups. Values in these regions were not different between groups.

### Seizures Result in an Acute Increase in Hippocampal IL-1β and S100B Levels

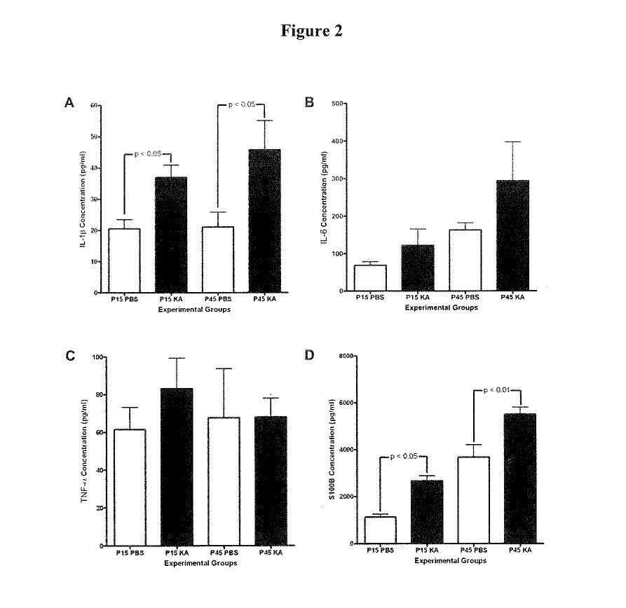

To determine whether seizures resulted in increases in pro-inflammatory cytokine levels and glial activation (data expressed as pg/ml±SEM; n), changes in levels of pro-inflammatory cytokines (IL-1β, IL-6, TNF-α, and S100), in hippocampal brain homogenates 24 hr following KA administration were measured (FIG. 2). This response in the immature (P15) and adult (P45) brain was compared. In the newborn, KA exposure resulted in a significant increase in IL-1β (37.0±4.0; n=7) compared to PBS-treated controls (20.5±3.0; n=6) (FIG. 2A). A similar increase was observed in the P45 animals treated with KA (45.8±9.3; n=9) compared to age matched controls (21.1±4.8; n=4). There were no significant differences between the immature and adult levels of IL-1β. IL-6 also showed a similar trend of changes as IL-β but did not reach statistical significance (FIG. 2B). There was no change in the level of TNF-α in the four groups (FIG. 2C). In contrast, S100B levels (FIG. 2D) significantly increased in KA-treated newborns (2664.0±225.8, n=7) compared to PBS-treated controls (1123±130.7, n=4). A similar increase was observed in P45 animals treated with KA (5508±304.3, n=10) compared to PBS-treated P45 controls (3678.0±529.3, n=4). S100B levels were significantly higher in adults compared to newborns both in KA-treated and PBS-treated groups.

### Early-Life Seizures Result in Neurobehavioral Impairment

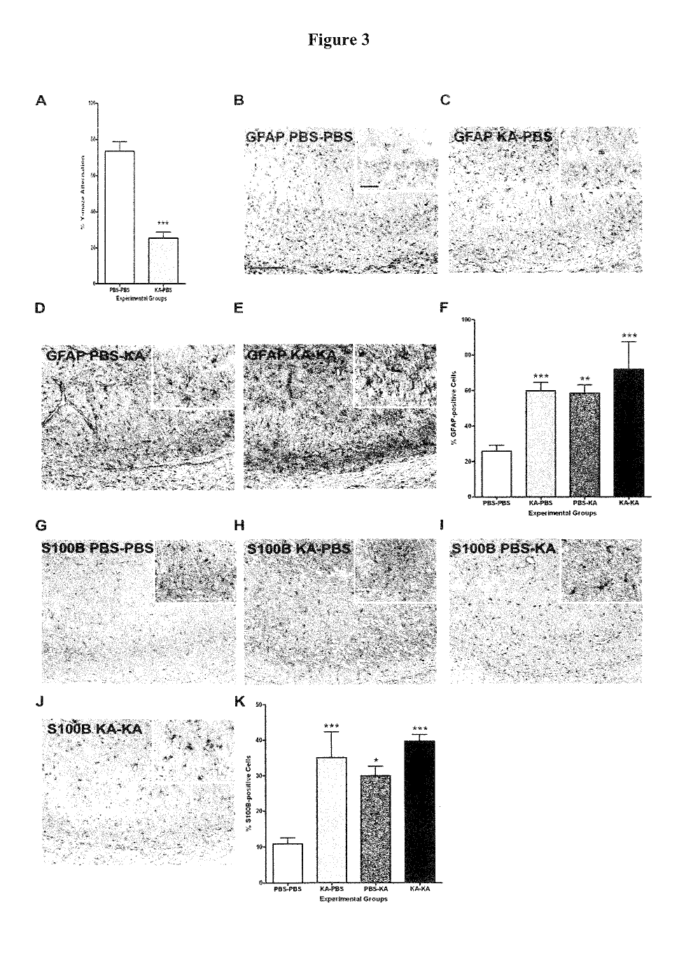

To determine whether the acute increase in IL-1β and S100B following KA-induced seizures in the newborn was accompanied by abnormal neurobehavioral outcome, animals were tested for the ability to perform in the Y-maze, a test for hippocampal-linked behavior (data expressed as % alternation±SEM; n) (FIG. 3A). Despite the absence of neuronal injury, animals in the early life seizure group showed significant impairment (KA-PBS, 25.4±3.4, n=12) compared to controls (PBS-1PBS, 73.4±5.3, n=8) after a 40-day recovery period.

### Seizures in the Newborn Brain Result in Prolonged Glial Activation

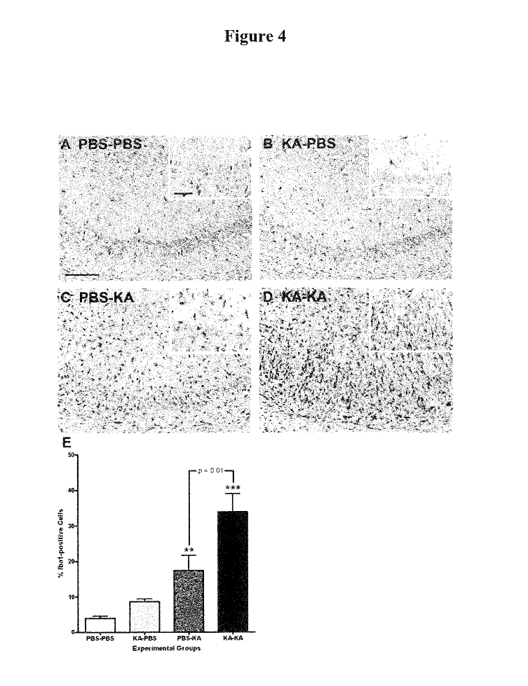

To determine whether seizures in the immature brain resulted in glial activation despite the absence of neuronal injury, immunohistochemical methods were used to quantify changes in astrocyte-specific markers, GFAP and S100B (FIGS. 3B-K) and the microglial marker, Ibal (FIG. 4) in the hippocampus. Immunostaining (data expressed as % of immuno-positive cells±SEM; n) in the four long-term (P55) recovery groups, control (PBS-PBS), immature seizures (KA-PBS), adult seizures (PBS-KA) and ‘two-hit’ (KA-KA) groups were compared.

A single administration of KA on P15 resulted in a significant increase in GFAP immunoreactivity in the hippocampus (FIGS. 3B-F), which persisted after 40 days recovery (KA-PBS, 59.9±4.8, n=10) compared to controls (PBS-PBS, 25.8±3.4, n=13). Animals in the ‘two-hit’ group showed significant increases in GFAP (KA-KA, 72.0±15.5, n=5) compared to controls but were not significantly different from the adult seizure group (PBS-KA, 50.3±6.5, n=10). GFAP-positive astrocytes in the KA-PBS, PBS-KA and KA-KA groups had thicker processes and enhanced staining compared to controls. These cells also tended to surround neurons. For every hippocampal region examined (CA1, CA2, CA3, DG and PoDG), all three KA-treated groups had significantly increased GFAP-positive cells compared to the PBS-PBS group. No individual region showed a higher percentage of GFAP-positive cells compared to other regions examined.

A similar response was observed for the glial-derived protein, S100B (FIGS. 3G-K). S100B immunoreactive astrocytes in the hippocampus were significantly increased 40 days after a single exposure to KA on P15 (KA-PBS, 35.0±7.3, n=10) compared to controls (PBS-PBS, 10.8±1.7, n=113). Again, animals in the ‘two-hit’ group showed significant increases in S100B (KA-KA, 39.6±1.9, n=6) compared to controls but were not significantly different from the adult seizure group (PBS-KA, 31.0±2.6, n=8). In the KA-exposed animals, the morphology of these cells was typically that of an activated astrocyte (FIGS. 3H-J).

### Microglial Activation is Transient and is Increased by a ‘Second Hit’

Notably, animals in the ‘two-hit’ group showed a significant increase (FIG. 4) in Ibal-immunoreactive microglia (KA-KA, 34.0±5.0, n=6) compared to the adult seizure group (PBSKA, 17.4±4.3, n=5). Ibal expression in both these groups (FIGS. 4C, D) was also significantly increased compared to controls (PBS-PBS, 4.0±0.6, n=6) (FIG. 4A). In contrast to the persistent increase in GFAP and S100B, activated microglia were not increased in the long-term recovery group (KA-PBS, 8.7±0.8, n=8) after early-life seizures (FIG. 4B).

### Increased Clusterin Levels Implicate Microglial Activation in the ‘Two-Hit’ Model

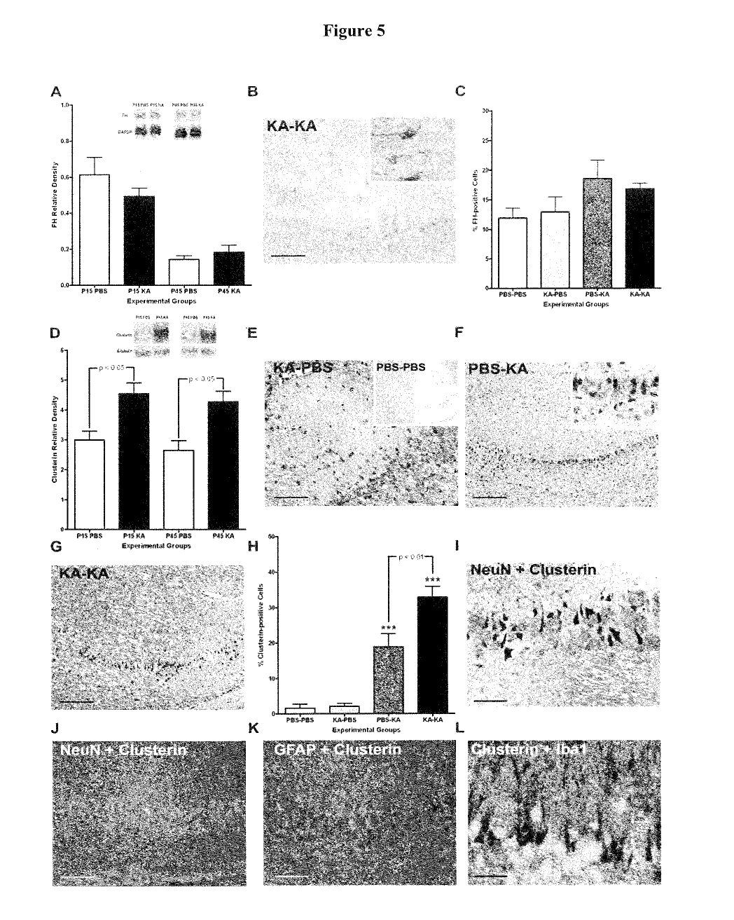

FH and clusterin modulate microglial activation and astrocyte-dependent cell death following brain injury (16-18, 28). Changes were measured in both proteins in the hippocampus 24-hr following seizures in the newborn (FIGS. 5A, D) using Western blotting (data expressed as relative density±SEM; n). There was no difference in FH levels between controls and KA-treated groups at P15 or P45 (FIG. 5A). Next, immunohistochemical methods were used to determine whether early-life seizures resulted in persistent changes in FH (FIGS. 5B, C) in the hippocampus in adulthood. There were no significant differences in values for FH in the long-term recovery groups. FH-immunoreactive cells had the morphology of astrocytes, and were also present in the frontal cortex, striatum, and periventricular areas.

After 24-hr recovery from KA-induced seizures, clusterin levels in the hippocampus were significantly higher both on P15 (4.5±0.36, n=8), and P45 (4.3±0.33, n=5) compared to age-matched controls (3.0±0.30, n=6) and (2.7±0.31, n=6) (FIG. 5D). Early-life seizures did not result in persistent changes in clusterin (FIGS. 7 E) in the hippocampus (data expressed as % of immuno-positive cells±SEM). Animals exposed to KA in adulthood (FIG. 5F) showed a significant increase in clusterin (PBS-KA, 18.9±3.7, n=7) compared to controls.

Clusterin levels were significantly increased in the ‘two-hit’ group (KA-KA, 32.9±3.1, n=6) compared to both adult seizures (PBS-KA, 18.9±3.7, n=7), early-life seizures (KA-PBS, 2.1±0.8, n=7) and controls (PBS-PBS, 1.6±1.1, n=8) (FIGS. 5G, H). To identify the cellular origin of clusterin, double-labeling studies were carried out in all 4 experimental groups (n=4 per group) (FIGS. 5I-L). Qualitative assessment of clusterin immunostaining showed clusterin positive cells in the entorhinal cortex, cerebral cortex, midbrain and hippocampal layers CA1, CA2, CA3 dentate gyrus and PoDG. Clusterin-positive cells did not co-label with NeuN (FIGS. 5I, J), or GFAP (FIG. 5K), but did co-label with Ibal (FIG. 5L), identifying them as microglia.

### Glial Glutamate Transporters GLAST and GLT-1 Increase Following Seizures

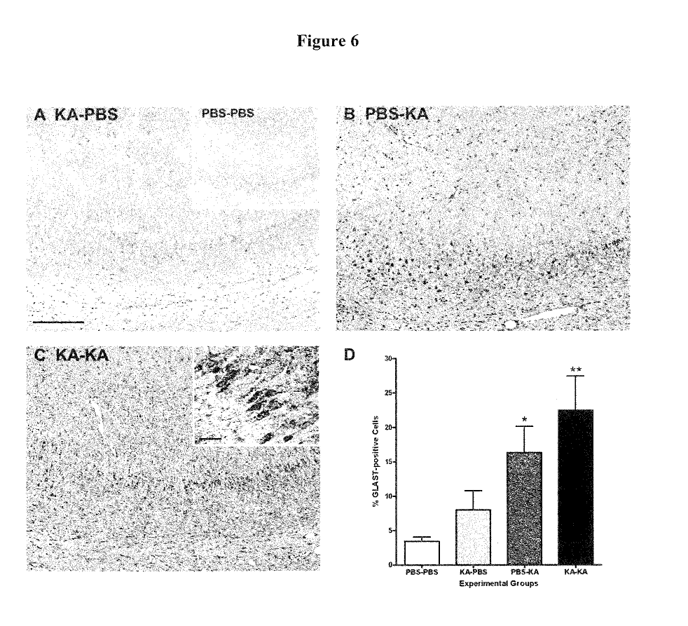

To determine one potential mechanism by which glial activation may result in neuronal dysfunction following early-life seizures, immunohistochemical methods were used to measure changes in astrocyte glutamate transporters GLAST and GLT-1 in the hippocampus (FIG. 6) (13). Expression of these proteins was compared in the four long-term experimental groups after exposure to KA or PBS on P15 (data expressed as % of immuno-positive cells±SEM; n). GLAST and GLT-1 immunoreactive cells showed a similar pericellular staining pattern and distribution in the brain (FIGS. 6A-C). The majority of labeled cells were distributed in the hippocampus, entorhinal cortex and frontal cortex. Neither GLAST nor GLT-1 expression was significantly increased over long-term recovery in the hippocampus following early-life seizures (FIG. 6D). In contrast, animals exposed to KA in adulthood showed a significant increase in GLAST (PBS-KA, 16.3±3.8, n=7) compared to controls (PBS-PBS; 3.4±0.6, n=7). GLAST levels were also significantly increased in the ‘two-hit’ group (KA-KA, 22.4±5.0, n=6) compared to controls. Similar results were observed for GLT-1. Values for GLT-1 in the early-life seizure group (KA-PBS, 5.9±1.9, n=6) were not significantly different compared to controls (PBS-PBS, 7.2±1.1, n=6), while animals exposed to KA in adulthood did show a significant increase in GLT-1 (PBS-KA, 19.4±3.5, n=10) compared to controls. GLT-1 levels were significantly increased in the ‘two-hit’ group (KA-KA, 25.0±4.2, n=5) compared to controls.

### Delayed Administration of Minozac Prevents Hippocampal-Dependent Behavioral Deficit Following KA-Induced Seizures

Remarkably, treatment with the small molecule compound Mzc after KA-induced seizures on P15 (FIG. 7A), prevented the compromise of behavioral function seen in the KA-exposed group (FIG. 7B). After 30 days recovery, the percent alternation in KA-exposed animals treated with saline (KA-Sal, 53.3±3.1, n=21) was significantly reduced compared to controls (PBS-Sal, 83.0±3.4, n=10). Animals treated with Mzc following KA-induced seizures (KA-Mzc, 79.5±2.5, n=20) showed significantly improved performance in this test compared to KA-Saline animals (P<0.001).

### Delayed Administration of Minozac Suppresses Kainate-Induced Acute Increase in Pro-Inflammatory Cytokines

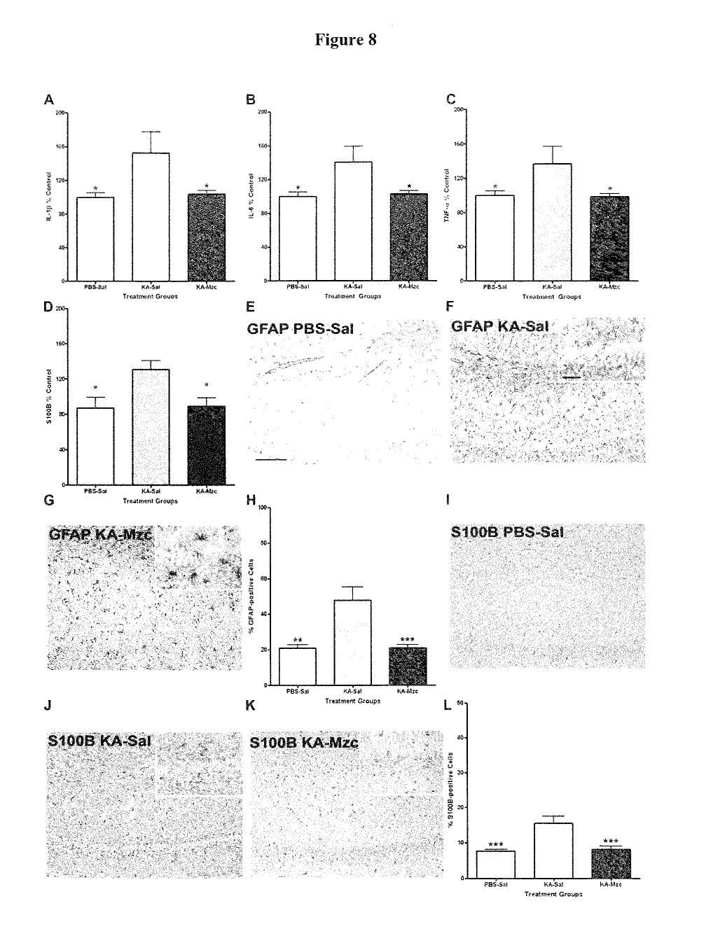

Determination as to whether the prevention by Mzc of neurobehavioral impairment after early-life seizures was the result of suppression of the acute cytokine, and long-term glial activation responses was sought next. Treatment with Mzc after KA-induced seizures on P15 also prevented the acute increase in pro-inflammatory cytokines (FIGS. 8A-D) in hippocampal brain homogenates (data expressed as % control±SEM, n). In the Mzc-treated group at 12 hr recovery after KA exposure, levels of IL-1β (103.7±4.6, n=12), IL-6 (103.2±3.8, n=12), TNF-α (98.5±3.9, n=12) and S100B (89.1±9.7, n=8) were not significantly different from controls and were significantly suppressed in comparison to the animals exposed to KA and treated with saline alone.

### Delayed Administration of Minozac Prevents KA—Induced Prolonged Increase in Glial Activation

Treatment with Mzc after KA exposure on P15 also prevented the long-term increase in astrocyte activation (FIGS. 8E-L). After 30 days recovery, glial activation was quantified by immunohistochemical methods. Levels of GFAP immunoreactive cells (FIGS. 8E-H) were significantly increased in KA-exposed animals (KA-Sal, 47.8±7.6, n=8), and this increase was prevented by treatment with Mzc (KA-Mzc, 20.9±1.8, n=9), (FIG. 8H). A similar result was seen for S1008 for both groups (KA-Sal, 15.6±2.0, n=12; KA-Mzc, 8.1±0.9, n=13), (FIGS. 8I-L).

### Discussion

This study addressed two questions, the dichotomy between neurobehavioral impairment in animals following early-life seizures despite the absence of neuronal injury and second, the mechanisms by which seizures in the immature brain may increase vulnerability to seizures later in life (4, 6, 29, 30). The key findings of this study are; (a) increased susceptibility to seizures in the ‘two-hit’ animals exposed to a ‘second-hit’ of KA following early-life seizures; (b) the longterm increase in glial activation and impairment of hippocampal-dependent behavior following early-life seizures; (c) enhanced microglial activation in the ‘two-hit’ animals exposed to KA on P15 and P45 and; (d) prevention of both outcomes by delayed administration of a small-molecule inhibitor of pro-inflammatory cytokine upregulation.

The lack of neuronal injury in the early-life seizure group and the increased injury in the ‘two-hit’ group are consistent with previous studies (2). The impairment in neurobehavioral function seen in this and other studies (31) following early-life seizures suggests a compromise of hippocampal neuronal function, although other limbic areas may also be injured (32). The prevention of this impairment by treatment with Mzc implicates pro-inflammatory cytokines in the initiation of the process. The lack of difference in the cytokine response in the early-life and adult seizure groups does not rule out age-dependent differences in this initial cytokine response as such differences have been reported (22) and these data reflect only a single time point. The prevention of the long-term increase in glial activation in the Mzc-treated animals demonstrates that glial activation can contribute to neuronal dysfunction (8).

The threshold for generation of seizures is lower in the immature than the adult brain (6) but the immature hippocampus is resistant to SE-induced structural damage (29). This discrepancy implies that the mechanisms by which early life seizures increase the vulnerability of the adult to seizures and neurologic injury occur at the molecular or subcellular level. Previous studies have implicated changes in AMPA receptors (33), glutamate receptor subunits (34), and the neuronal glutamate transporter, EAAC1 (15). The data showing enhanced microglial activation in the ‘two-hit’ group implicates glial (astrocytes and microglial) activation in the mechanisms leading to increased susceptibility to seizures and neurologic injury following early life seizures. This hypothesis is supported by the improved behavioral function and prevention of long-term glial activation in the Mzc-treated animals. The precise mechanisms by which activated astrocytes and microglia cause neurologic injury remain obscure (35, 36).

Neuroinflammation is a well-established response to central nervous system injury (36) including epilepsy (37) although the potential of this response as a therapeutic target in epilepsy has not been explored in detail. Precedent from human pathologic, in vitro, and in vivo studies of Alzheimer's disease have implicated a glia-mediated neuroinflammatory response both in the pathophysiology of the disease (8) and as treatment target (19, 26, 38). Microglial activation leading to overexpression of IL-1 has been proposed as the pivotal step in initiating a self propagating cytokine cycle culminating in neurodegeneration (8, 39). The data showing an early increase in IL-1β is consistent with previous studies of pro-inflammatory cytokine mRNA changes in response to KA on P15 (22) and P21 (40). The pivotal role of pro-inflammatory cytokines in the initiation of this cycle is supported by the therapeutic benefit afforded in the Mzc-treated early-life seizure group in which the acute cytokine response was suppressed. IL-1β and pro-inflammatory cytokines may also function in epilepsy as pro-convulsant signaling molecules independent of such a cycle (41).

Evidence has been found of long-term glial activation in the hippocampi of rats exposed to KA on P15, manifest as increases in GFAP and S100B (42, 43) expression after 40 days recovery. The astrocyte-derived cytokine S100B was increased both acutely and during long-term recovery in this study. Together with the long-term increase in GFAP, this finding implies that astrocytes remain activated following early-life seizures. While the mechanisms by which S100B increases vulnerability to neurologic injury remain to be determined, studies of mice overexpressing S1OOB demonstrate both an increase in glial activation and increased susceptibility to neurologic insults (10, 44). Increased S100B levels found in the adult control animals is consistent with the rise in S100B associated with aging (45).

There was no long-term change in the expression of the astrocyte glutamate transporters, which are responsible for the majority of glutamate uptake in the central nervous system (13, 14). The increase in both transporters in the ‘two-hit’ group was comparable to that found in the adult (PBS-KA) seizure group, and may be a protective response (46). These data imply that, either the astrocyte glutamate transporters do not contribute to the increase in seizure susceptibility following early life seizures, or the functional properties of these transporters are altered.

Clusterin expression is upregulated in astrocytes following seizures (18). The increase in clusterin in the KA-exposed animals is of microglial origin. This result supports the finding that microglial activation is potentiated in the ‘two-hit’ group and may be a mechanism by which activated microglia can lead to cell injury (17). The complement inhibitory protein, Factor H (28), acts as a chemotactic factor for activated microglia in amyloid-β-induced brain injury (16). Lack of change in Factor H levels in response to KA suggests that this signaling pathway is not involved in microglial recruitment after seizures.

The small molecule used in these studies, Mzc (19), is a bioavailable, water-soluble, CNS-penetrant, non-toxic compound that inhibits hippocampal pro-inflammatory cytokine upregulation, suppresses synaptic dysfunction, and attenuates hippocampal-dependent behavioral deficits in an Alzheimer's disease mouse model. The results presented here and the ability to produce Mzc in large scale under FDA guidelines (19) indicates the need to investigate the potential of this compound as a novel therapeutic in pediatric epilepsy. Efficacy in the present study lends further support to the hypothesis that targeting neuroinflammation may alter progression of central nervous system disorders (19, 20, 38).

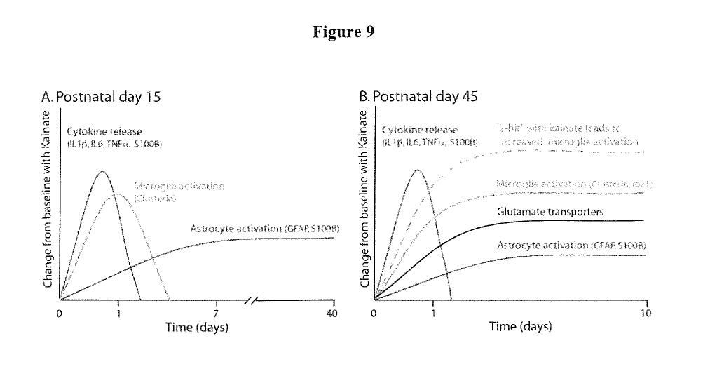

These data implicate neuroinflammation both in the chronic neurologic sequelae of early-life seizures, and the increased susceptibility to further neurologic injury following seizures in the newborn period. A model (FIG. 9) is proposed in which an initial rise in pro-inflammatory cytokines following early life seizures, particularly IL-1β and S100B, results in further activation of microglia. This activation is also transient, but is followed by a persistent increase in astrocyte activation. Activated astrocytes result in neurobehavioral impairment, and may increase the susceptibility to a second neurologic insult in part through enhanced microglial activation after the insult. Early-life seizures prime microglia for an exaggerated response to a second neurologic insult (20). These hypotheses are supported by the effects of suppression of the initial pro-inflammatory cytokine response, the regional specificity of the glial activation response to the hippocampus, previous data showing increased susceptibility to neurologic injury in transgenic mice overexpressing S100B (10, 44), and a similar model proposed for neurodegeneration in Alzheimer's disease (7, 8, 19, 20).

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methods etc. which are reported therein which might be used in connection with the invention. Nothing herein is to be construed as an admission that the invention is not entitled to antedate such disclosure by virtue of prior invention.

TABLE 1

| Compound | Compound | Synthetic |
|---|---|---|
| Number | Structure | Code |
| 1 | 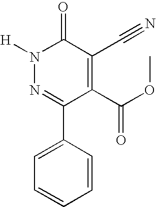 | MW01-ES1 |
| 4 | 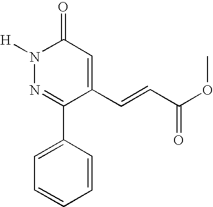 | MW01-ES112 |
| 10 | 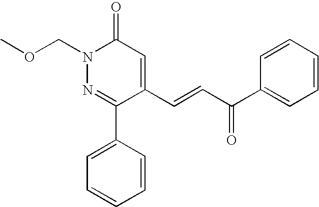 | MW01-ES159 |
| 11 | 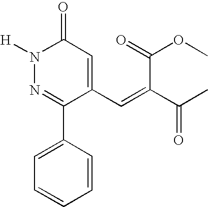 | MW01-ES21 |
| 12 | 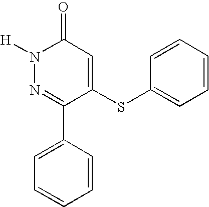 | MW01-ES31 |
| 13 | 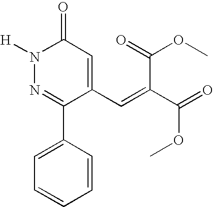 | MW01-ES60 |
| 14 | 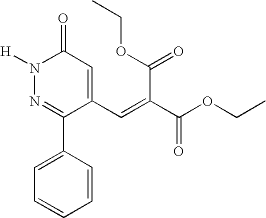 | MW01-ES61 |
| 16 | 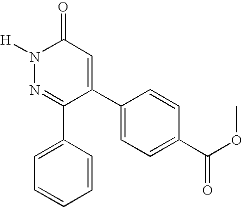 | MW01-ES75 |
| 17 | 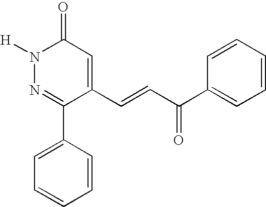 | MW01-ES81 |
| 18 | 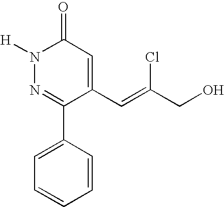 | MW01-ES91 |
| 20 | 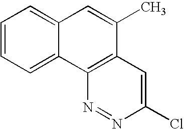 | MW01-1-04- L-D04 |
| 23 | 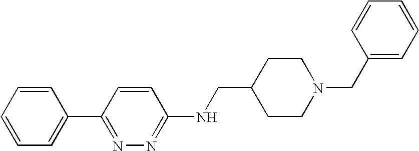 | MW01-1-15- L-H07 |
| 24 | 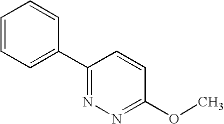 | MW01-1-16- L-F05 |
| 25 | 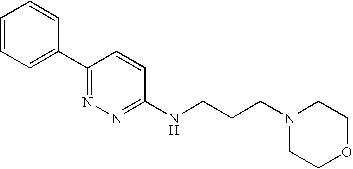 | MW01-1-18- L-B09 |
| 31 | 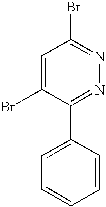 | MW01-1- 035LKM |
| 40 | 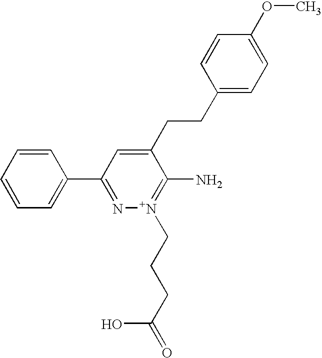 | MW01-1-09- L-G07 |
| 41 | 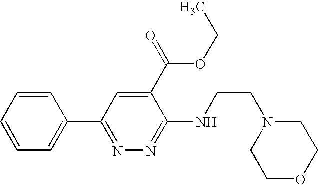 | MW01-2-03- L-C02 |
| 43 | 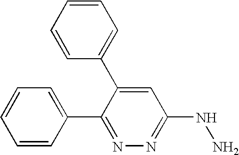 | MW01-1-15- L-E09 |
| 44 | 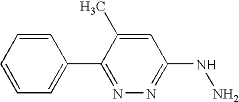 | MW01-1-16- L-B11 |
| 47 | 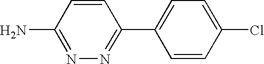 | MW01-4- 198B-Z |
| 48 | 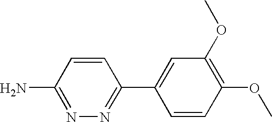 | MW01-5- 144A-Z |
| 49 | 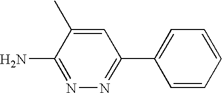 | MW01-4- 198C-Z |
| 50 | 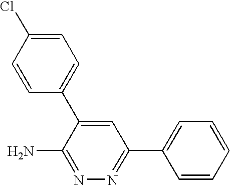 | MW01-5- 144C-Z |
| 51 | 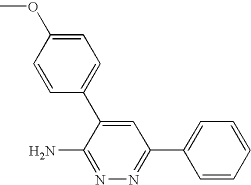 | MW01-5- 144D-Z |
| 52 | 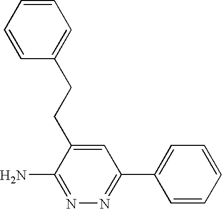 | MW01-5- 145A-Z |
| 54 | 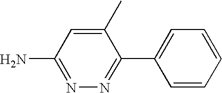 | MW01-5- 189Z |
| 55 | 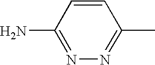 | MW01-5- 202B-Z |
| 61 | 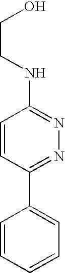 | MW01-1-01- L-D06 |
| 65 | 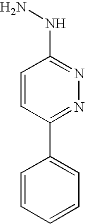 | MW01-1-01- L-E10 |
| 66 | 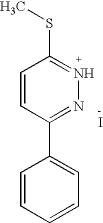 | MW01-1-02- L-E08 |
| 70 | 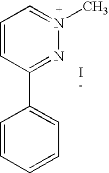 | MW01-1-03- L-D03 |
| 71 | 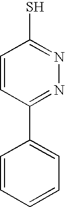 | MW01-1-03- L-F03 |
| 73 | 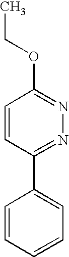 | MW01-1-03- L-G10 |
| 74 | 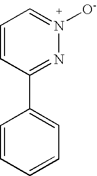 | MW01-1-03- L-H06 |
| 75 | 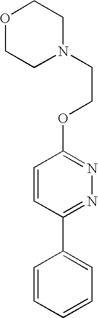 | MW01-1-04- L-C03 |
| 76 | 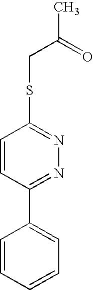 | MW01-1-07- L-H04 |
| 88 | 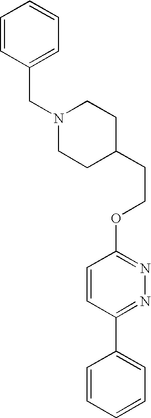 | MW01-1-100- L-A04 |
| 89 | 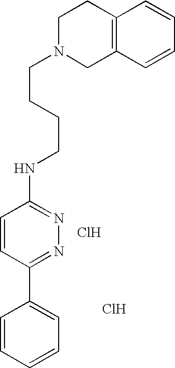 | MW01-1-100- L-A05 |
| 90 | 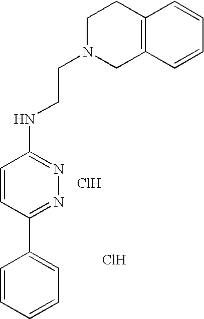 | MW01-1-100- L-A08 |
| 91 | 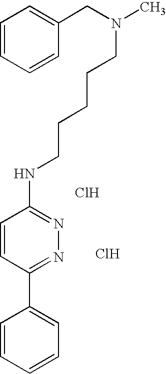 | MW01-1-100- L-A09 |
| 92 | 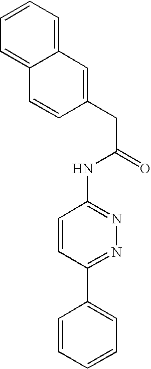 | MW01-1-11- L-E08 |
| 94 | 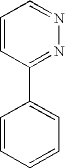 | MW01-1-15- L-G09 |
| 97 | 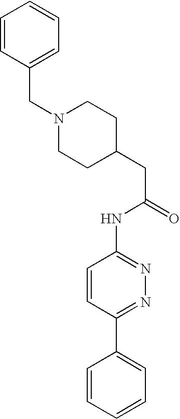 | MW01-1-16- L-G03 |
| 106 | 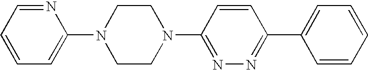 | MW01-9- 039Z |
| 107 | 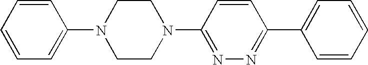 | MW01-9- 040Z |
| 108 | 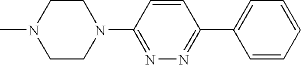 | MW01-9- 041Z |
| 109 | 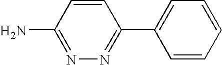 | MW01-9- 104A-Z |
| 110 | 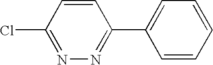 | MW01-9- 105A-Z |
| 111 | 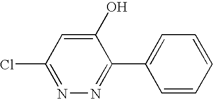 | MW01-9- 110A-Z |
| 112 | 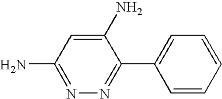 | MW01-9- 133A-Z |
| 113 | 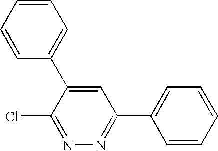 | MW01-9- 149A-Z |
| 114 | 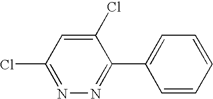 | MW01-9- 159A-Z |
| 115 | 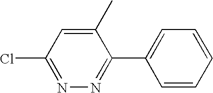 | MW01-9- 171Z |
| 116 | 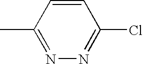 | MW01-9- 172Z |
| 118 | 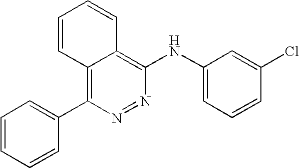 | MW01-9- 204Z |
| 120 | 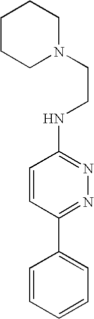 | MW01-1-16- L-G08 |
| 122 | 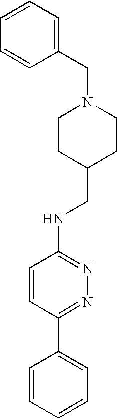 | MW01-1-17- L-G05 |
| 123 | 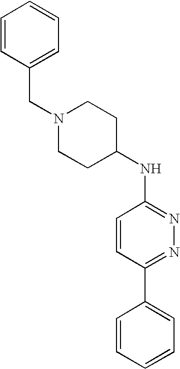 | MW01-1-17- L-G11 |
| 125 | 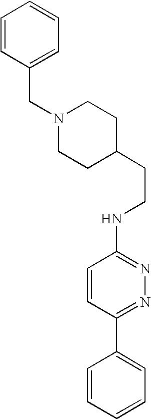 | MW01-1-17- L-H03 |
| 127 | 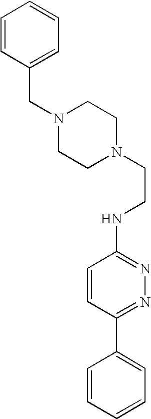 | MW01-1-17- L-H11 |
| 130 | 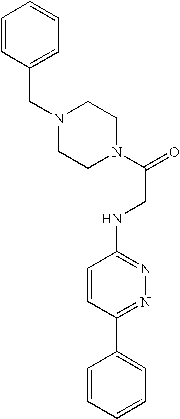 | MW01-1-18- L-A08 |
| 137 | 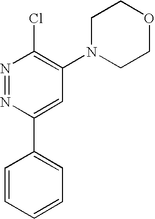 | MW01-2- 020SRM |
| 143 | 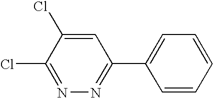 | MW01-2- 056WH |
| 149 | 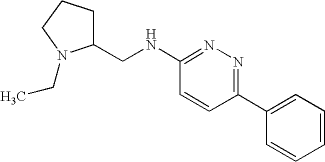 | MW01-1-18- L-A11 |
| 150 | 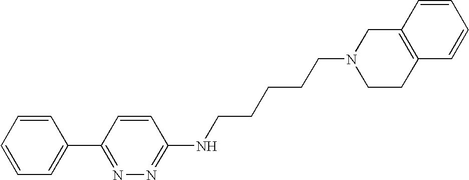 | MW01-1-18- L-B03 |
| 151 | 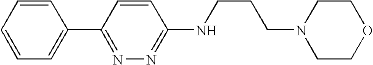 | MW01-1- 18-L-B09 |
| 158 | 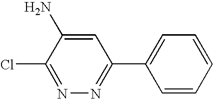 | MW01-3- 033WH |
| 159 | 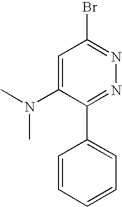 | MW01-3- 009WH |
| 173 | 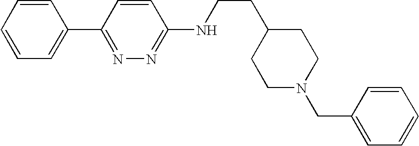 | MW01-2-03- L-D02 |
| 175 | 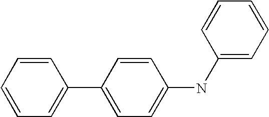 | MW01-2-06- L-F04 |
| 175A | 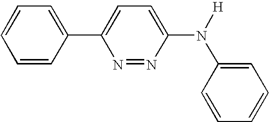 | |
| 179 | 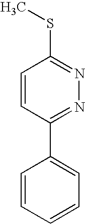 | MW01-2-33- L-B02 |
| 180 | 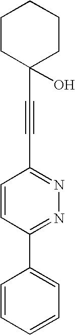 | MW01-3-01- L-G07 |
| 183 | 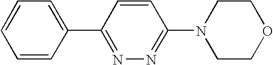 | MW01-5- 160WH |
| 184 | 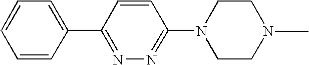 | MW01-5- 161WH |
| 189 | 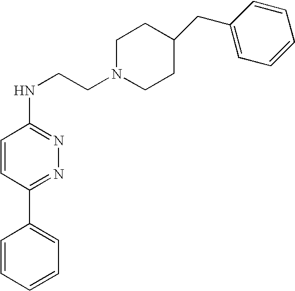 | MW01-6- 041WH |
| 190 | 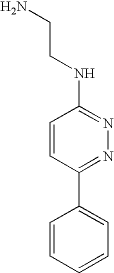 | MW01-6- 044WH |
| 192 | 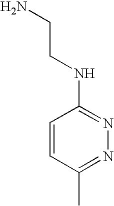 | MW01-6- 050WH |
| 197 | 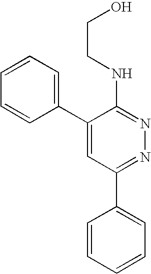 | MW01-1-01- L-A10 |
| 198 | 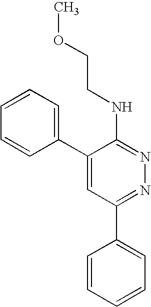 | MW01-1-01- L-B03 |
| 199 | 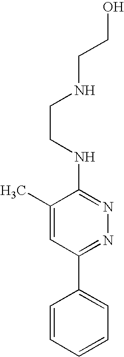 | MW01-1-01- L-B09 |
| 201 | 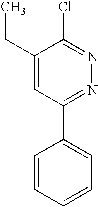 | MW01-1-01- L-E03 |
| 202 | 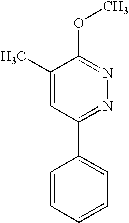 | MW01-1-01- L-E04 |
| 205 | 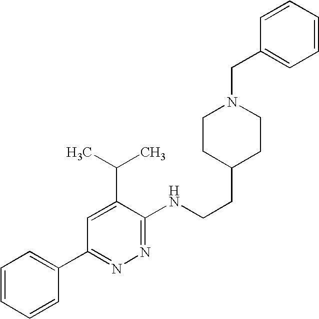 | MW01-1-18- L-B07 |
| 208 | 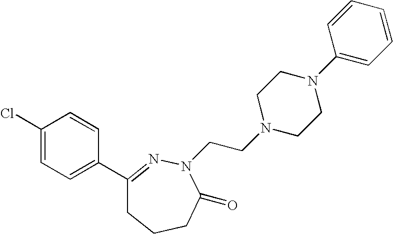 | MW01-1-03- L-G03 |
| 210 | 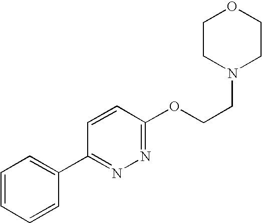 | MW01-1-04- L-C03 |
| 217 | 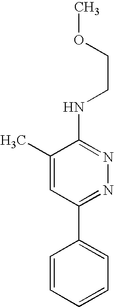 | MW01-1-02- L-E03 |
| 218 | 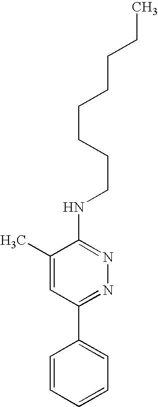 | MW01-1-02- L-E06 |
| 221 | 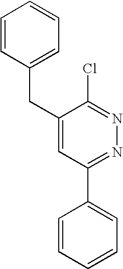 | MW01-1-02- L-F02 |
| 223 | 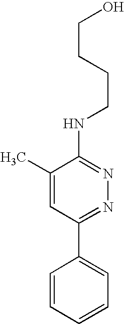 | MW01-1-02- L-F08 |
| 225 | 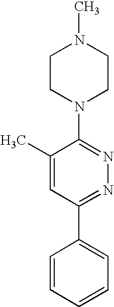 | MW01-1-02- L-G05 |
| 226 | 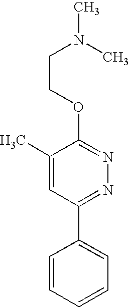 | MW01-1-02- L-G06 |
| 227 | 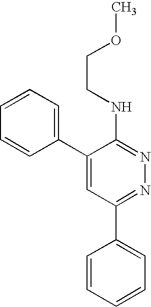 | MW01-1-03- L-A02 |
| 229 | 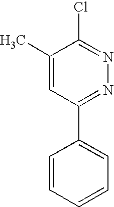 | MW01-1-03- L-B09 |
| 230 | 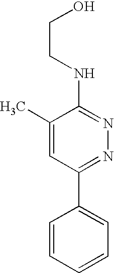 | MW01-1-03- L-B10 |
| 231 | 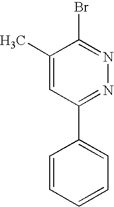 | MW01-1-03- L-C03 |
| 233 | 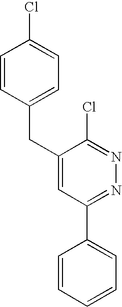 | MW01-1-03- L-C08 |
| 235 | 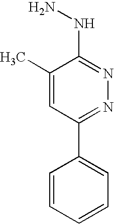 | MW01-1-03- L-E08 |
| 236 | 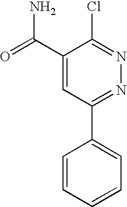 | MW01-1-03- L-E09 |
| 240 | 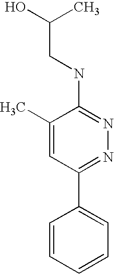 | MW01-1-04- L-A06 |
| 242 | 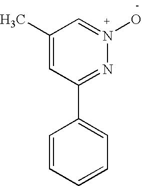 | MW01-1-04- L-D10 |
| 250 | 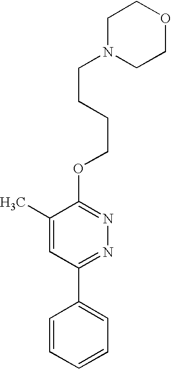 | MW01-1-05- L-B11 |
| 251 | 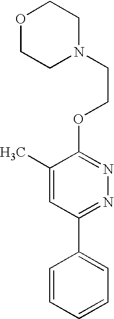 | MW01-1-05- L-C02 |
| 254 | 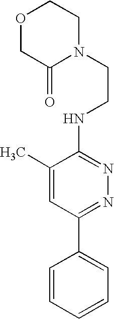 | MW01-1-05- L-G11 |
| 255 | 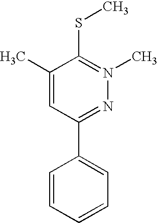 | MW01-1-05- L-H05 |
| 266 | 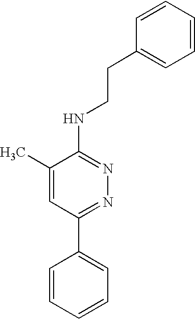 | MW01-1-08- L-D09 |
| 268 | 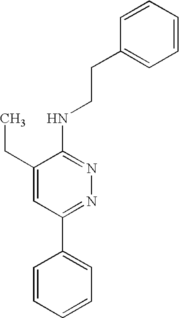 | MW01-1-09- L-C06 |
| 270 | 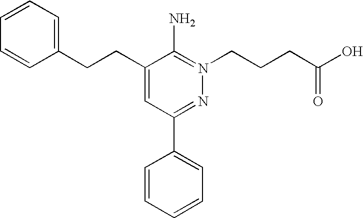 | MW01-1-09- L-G05 |
| 271 | 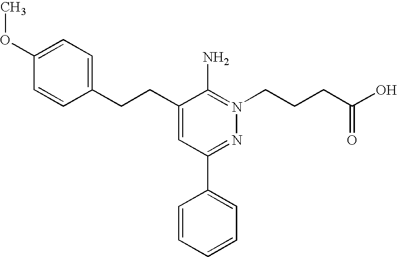 | MW01-1-09- L-G07 |
| 272 | 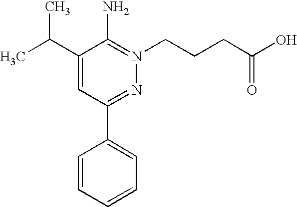 | MW01-1-09- L-G09 |
| 274 | 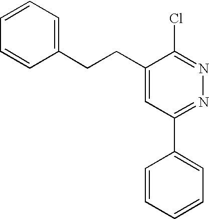 | MW01-1-09- L-H07 |
| 275 | 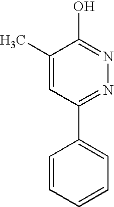 | MW01-1-15- L-A04 |
| 276 | 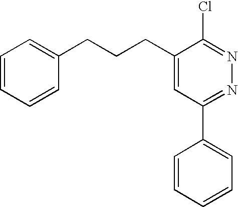 | MW01-1-15- L-B02 |
| 278 | 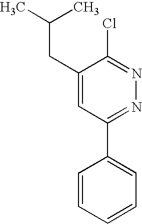 | MW01-1-15- L-B10 |
| 280 | 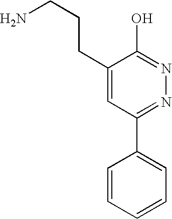 | MW01-1-15- L-C04 |
| 282 | 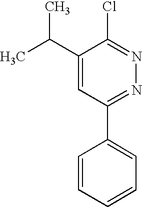 | MW01-1-15- L-D03 |
| 284 | 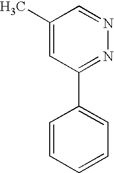 | MW01-1-15- L-G10 |
| 292 | 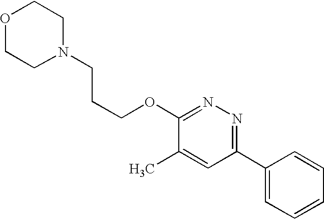 | MW01-1-17- L-A09 |
| 293 | 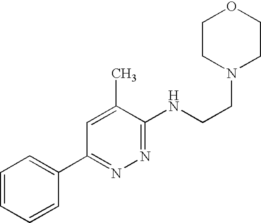 | MW01-1-17- L-A11 |
| 294 | 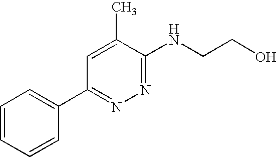 | MW01-1-17- L-B02 |
| 295 | 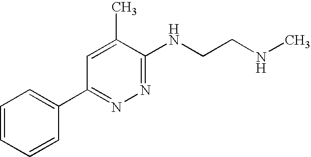 | MW01-1-17- L-B10 |
| 296 | 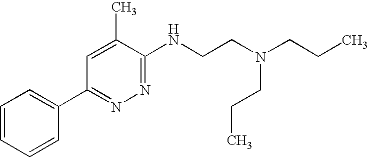 | MW01-1-17- L-E11 |
| 297 | 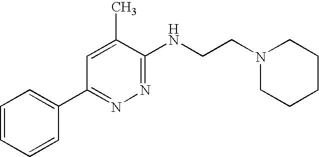 | MW01-1-17- L-F03 |
| 298 | 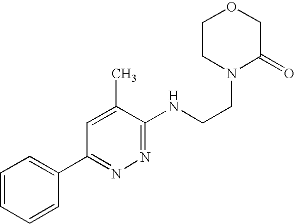 | MW01-1-17- L-H05 |
| 299 | 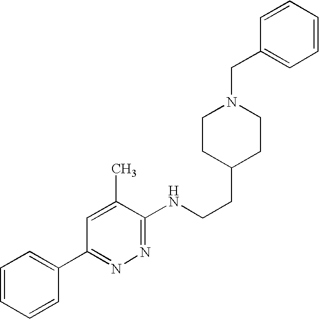 | MW01-1-18- L-A09 |
| 308 | 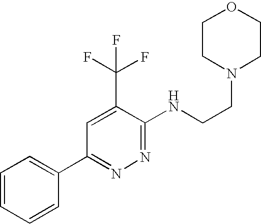 | MW01-2-03- L-B08 |
| 310 | 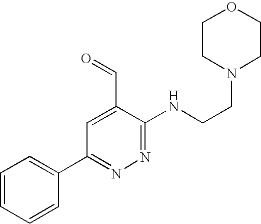 | MW01-2-03- L-C05 |
| 313 | 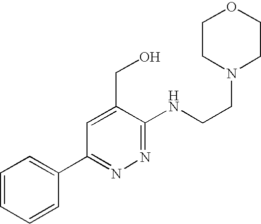 | MW01-2-03- L-G07 |
| 318 | 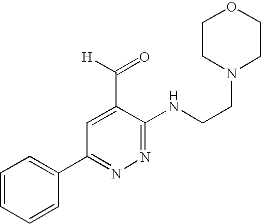 | MW01-2-101- L-H08 |
| 319 | 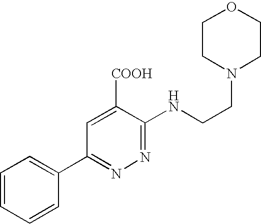 | MW01-2-10- L-E05 |
| 320 | 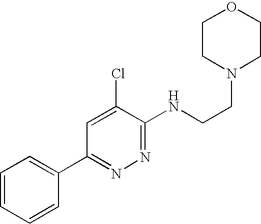 | MW01-2-10- L-E06 |
| 321 | 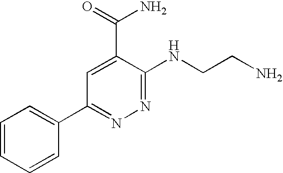 | MW01-2-20- L-B02 |
| 323 | 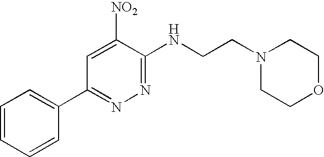 | MW01-2-20- L-D05 |
| 324 | 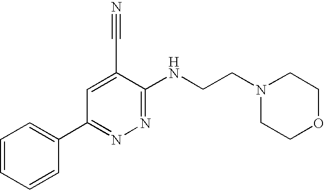 | MW01-2-20- L-E09 |
| 326 | 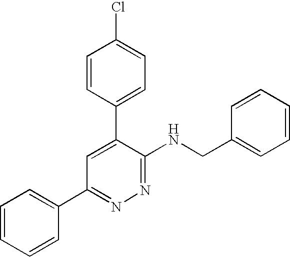 | MW01-2-25- L-H06 |
| 328 | 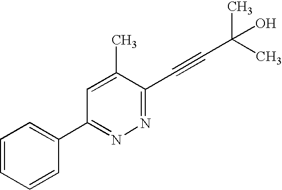 | MW01-3-01- L-G03 |
| 329 | 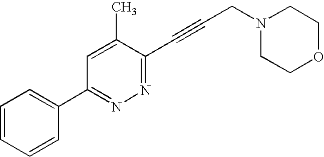 | MW01-3-01- L-G04 |
| 331 | 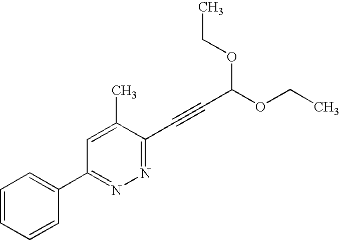 | MW01-3-01- L-G08 |
| 332 | 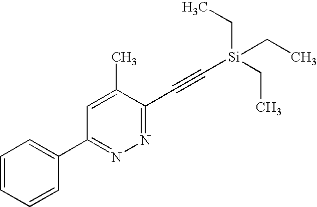 | MW01-3-01- L-G09 |
| 335 | 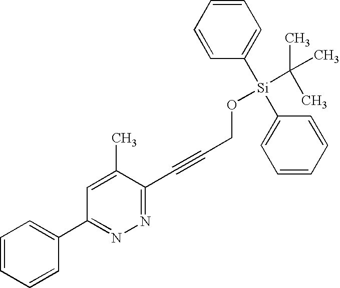 | MW01-3-06- L-E09 |
| 337 | 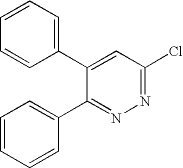 | MW01-1-07- L-G07 |
| 339 | 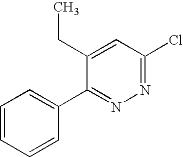 | MW01-1-15- L-C11 |
| 340 | 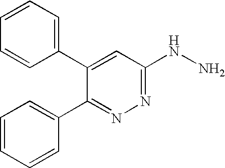 | MW01-1-15- L-E09 |
| 341 | 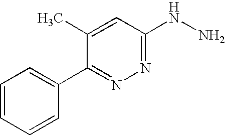 | MW01-1-16- L-B11 |
| 346 | 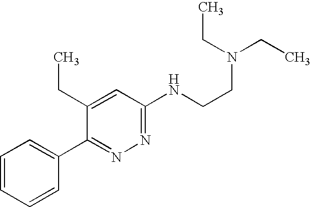 | MW01-1-17- L-F10 |
| 347 | 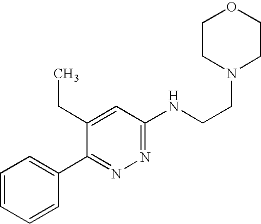 | MW01-1-17- L-F11 |
| 350 | 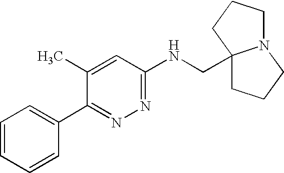 | MW01-2-20- L-B11 |
| 352 | 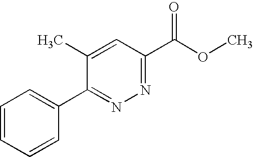 | MW01-3-01- L-F09 |
| 359 | 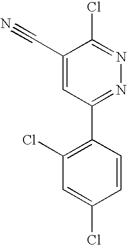 | MW01-1-03- L-E05 |
| 360 | 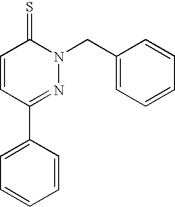 | MW01-1-03- L-A08 |
| 361 | 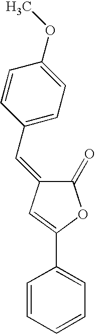 | MW01-1-03- L-H08 |
| 362 | 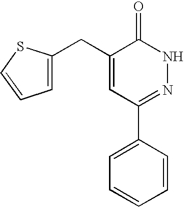 | MW01-1-01- L-H04 |
| 363 | 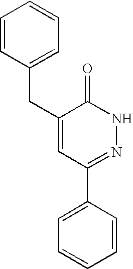 | MW01-1-01- L-H06 |
| 366 | 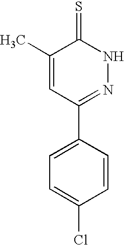 | MW01-1-03- L-E07 |
| 367 | 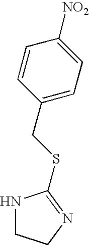 | MW01-1-05- L-E05 |
| 368 | 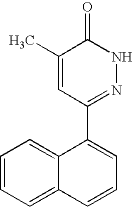 | MW01-1-03- L-B03 |
| 371 | 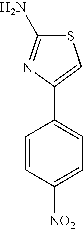 | MW01-1-05- L-E07 |
| 372 | 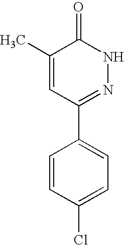 | MW01-1-03- L-A03 |
| 373 | 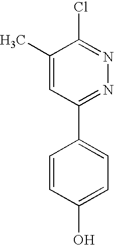 | MW01-1-03- L-E03 |
| 374 | 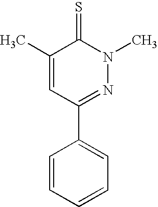 | MW01-1-01- L-H10 |
| 375 | 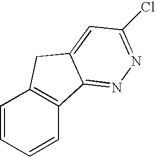 | MW01-1-04- L-H08 |
| 376 | 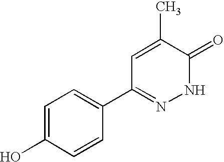 | MW01-1-01- L-G10 |
| 377 | 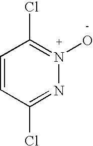 | MW01-1-03- L-G11 |
| 380 | 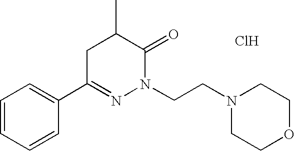 | MW01-1-04- L-B07 |
| 381 | 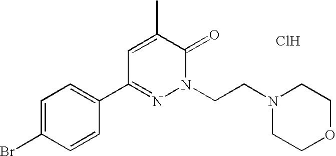 | MW01-1-04- L-C09 |
| 382 | 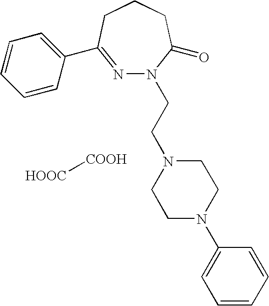 | MW01-1-10- L-G05 |

TABLE 2

| Compound | Compound | Synthetic |
|---|---|---|
| Number | Structure | Code |
| 22 | 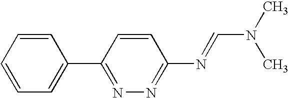 | MW01-1-15- L-E08 |
| 26 | 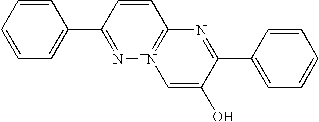 | MW01-2-02- L-H09 |
| 29 | 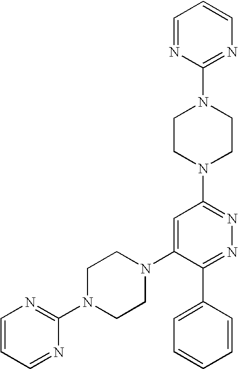 | MW01-1- 030A-LKM |
| 30 | 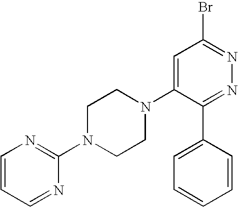 | MW01-1- 030B-LKM |
| 32 | 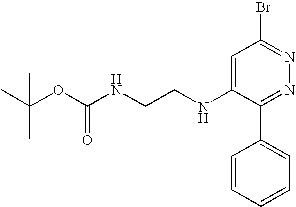 | MW01-1- 048AB-LKM |
| 33 | 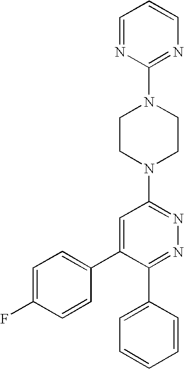 | MW01-2- 065LKM |
| 34 | 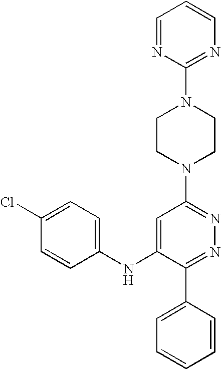 | MW01-2- 127LKM |
| 35 | 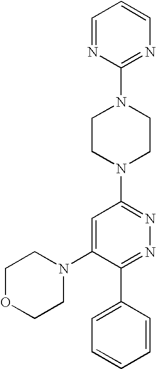 | MW01-2- 134LKM |
| 36 | 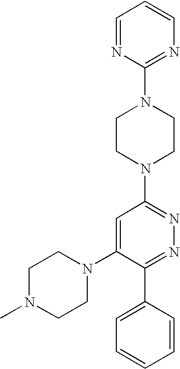 | MW01-2- 146LKM |
| 37 | 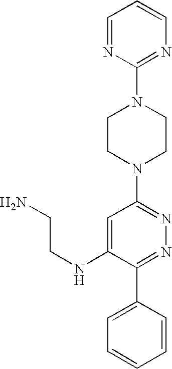 | MW01-2- 147LKM |
| 38 | 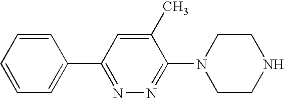 | MW01-1-02- L-B11 |
| 39 | 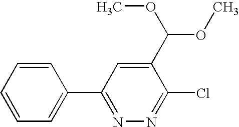 | MW01-1-04- L-F10 |
| 42 | 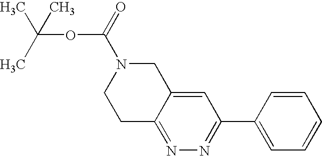 | MW01-2-33- L-A11 |
| 45 | 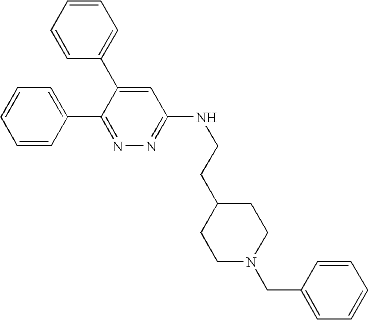 | MW01-1-17- L-E06 |
| 46 | 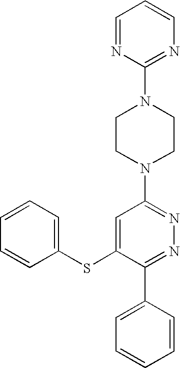 | MW01-1- 045MAS |
| 53 | 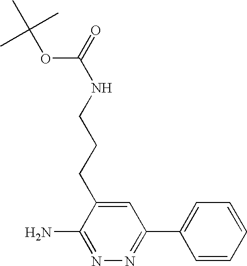 | MW01-5- 145B-Z |
| 56 | 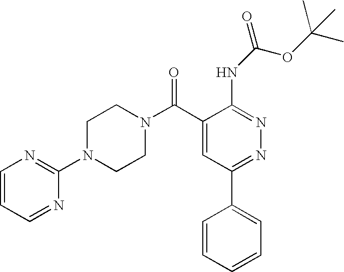 | MW01-7- 127AB-Z |
| 60 | 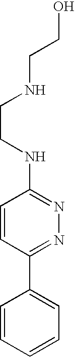 | MW01-1-01- L-B04 |
| 62 | 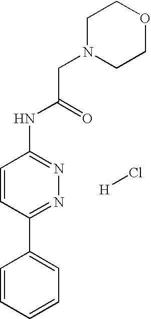 | MW01-1-01- L-D10 |
| 63 | 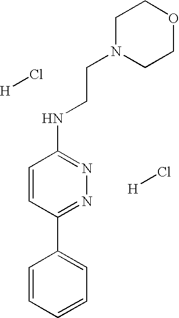 | MW01-1-01- L-E02 |
| 64 | 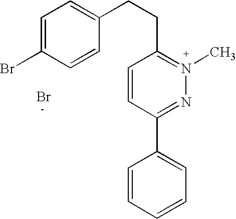 | MW01-1-01- L-E08 |
| 67 | 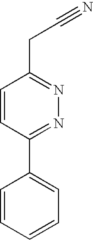 | MW01-1-02- L-H10 |
| 68 | 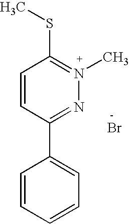 | MW01-1-03- L-A05 |
| 69 | 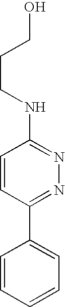 | MW01-1-03- L-B08 |
| 72 | 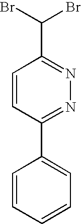 | MW01-1-03- L-G09 |
| 87 | 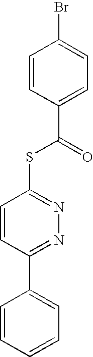 | MW01-1-08- L-E11 |
| 93 | 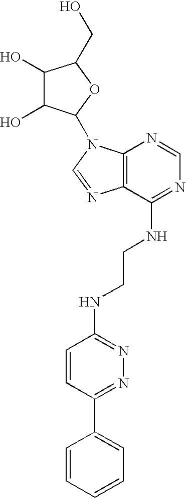 | MW01-1-13- L-G06 |
| 95 | 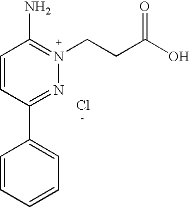 | MW01-1-16- L-D09 |
| 96 | 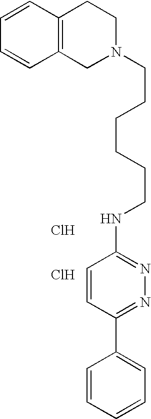 | MW01-1-16- L-E02 |
| 105 | 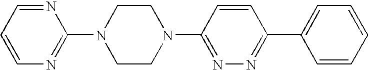 | MW01-9- 038Z |
| 121 | 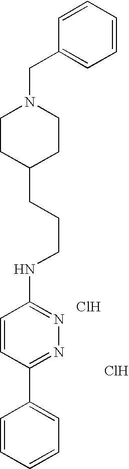 | MW01-1-17- L-G04 |
| 124 | 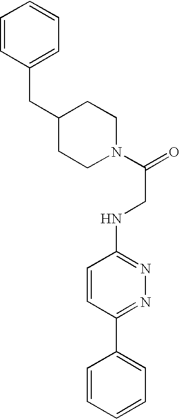 | MW01-1-17- L-H02 |
| 126 | 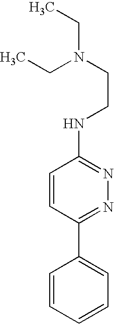 | MW01-1-17- L-H07 |
| 128 | 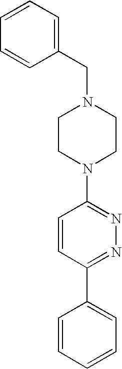 | MW01-1-18- L-A02 |
| 129 | 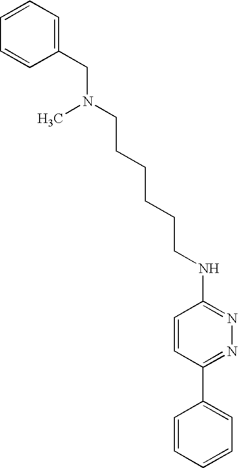 | MW01-1-18- L-A03 |
| 136 | 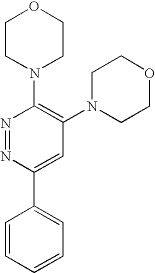 | MW01-2- 018SRM |
| 138 | 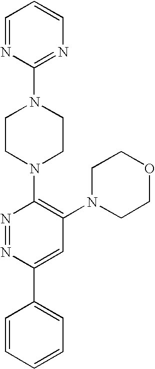 | MW01-2- 023SRM |
| 147 | 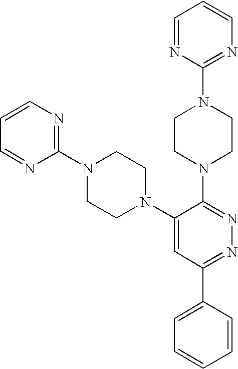 | MW01-2- 177A-WH |
| 148 | 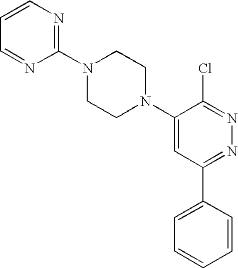 | MW01-2- 177B-WH |
| 153 | 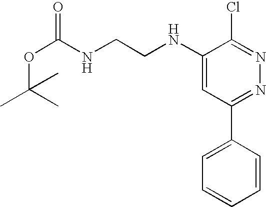 | MW01-2- 184WH |
| 155 | 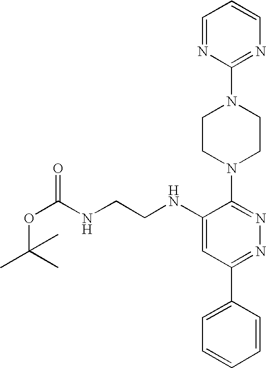 | MW01-2- 191A-WH |
| 156 | 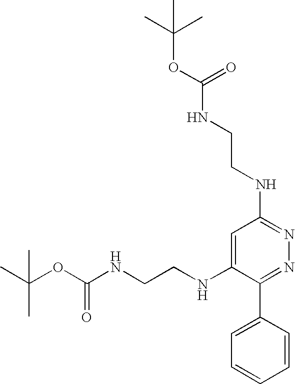 | MW01-2- 193B-WH |
| 157 | 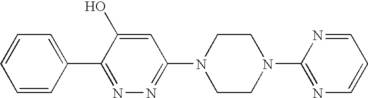 | MW01-3- 003WH |
| 160 | 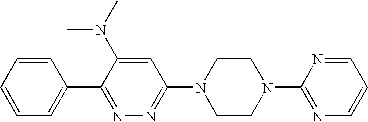 | MW01-3- 019A-WH |
| 161 | 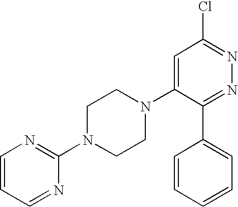 | MW01-3- 060A-WH |
| 162 | 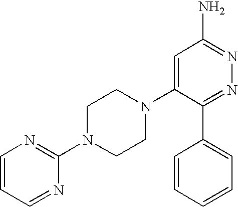 | MW01-3- 072WH |
| 163 | 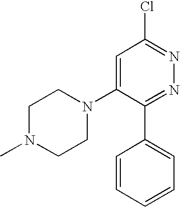 | MW01-3- 117WH |
| 164 | 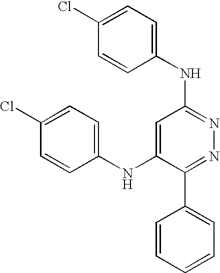 | MW01-3- 118WH |
| 166 | 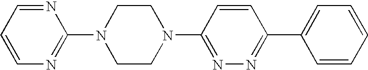 | MW01-3- 183WH |
| 171 | 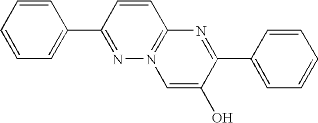 | MW01-2-03- L-G03 |
| 172 | 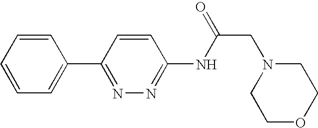 | MW01-2-03- L-C04 |
| 174 | 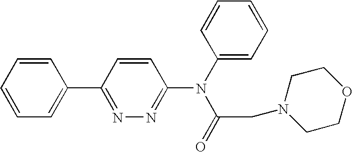 | MW01-2-03- L-G03 |
| 176 | 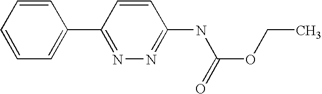 | MW01-2- 102-L-C11 |
| 177 | 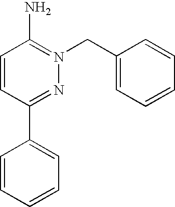 | MW01-2-21- L-F04 |
| 178 | 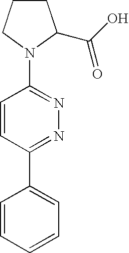 | MW01-2-24- L-G09 |
| 181A | 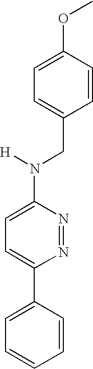 | |
| 186 | 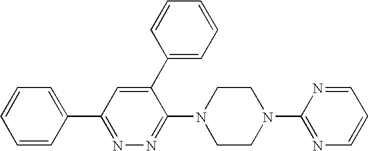 | MW01-5- 188WH |
| 188 | 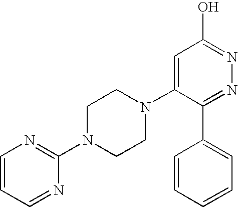 | MW01-6- 003WH |
| 191 | 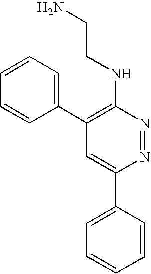 | MW01-6- 046WH |
| 200 | 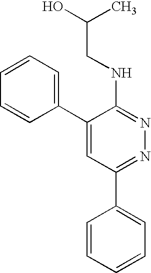 | MW01-1-01- L-C06 |
| 203 | 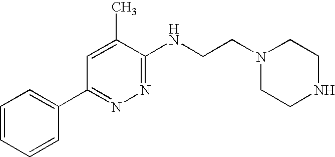 | MW01-2-03- L-D09 |
| 204 | 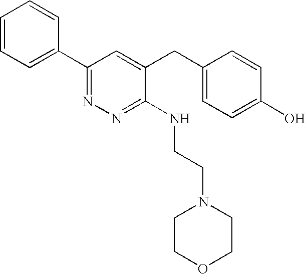 | MW01-1-01- L-B02 |
| 206 | 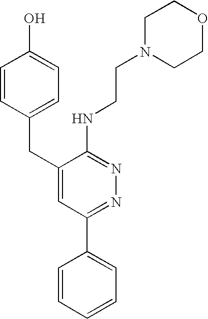 | MW01-2-03- L-D09 |
| 207 | 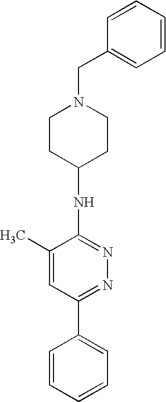 | MW01-2-03- L-G04 |
| 209 | 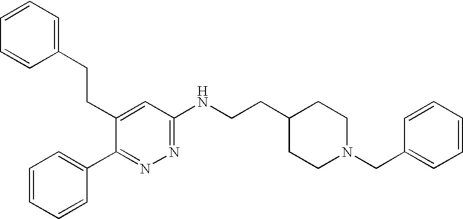 | MW01-1-17- L-E05 |
| 211 | 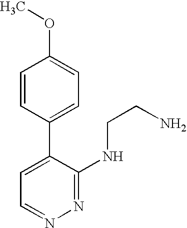 | MW01-1-04- L-C03 |
| 212 | 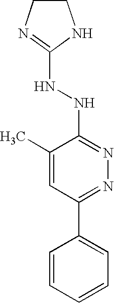 | MW01-1-01- L-E11 |
| 213 | 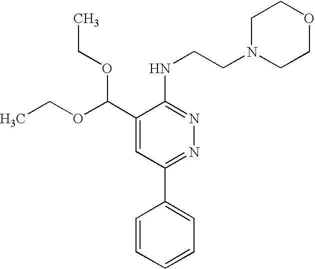 | MW01-1-01- L-F02 |
| 214 | 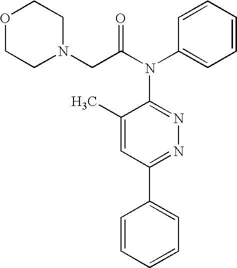 | MW01-1-01- L-F03 |
| 215 | 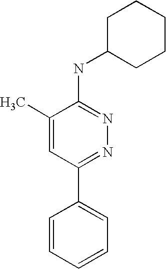 | MW01-1-01- L-G08 |
| 216 | 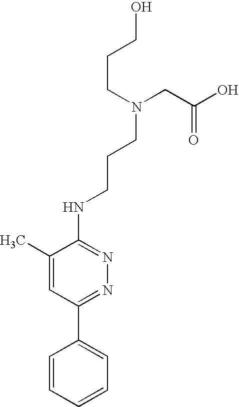 | MW01-1-02- L-D11 |
| 219 | 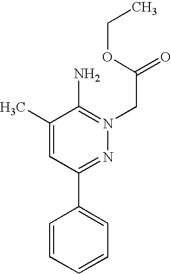 | MW01-1-02- L-E04 |
| 220 | 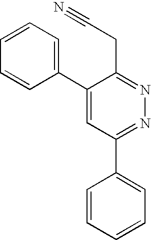 | MW01-1-02- L-E11 |
| 222 | 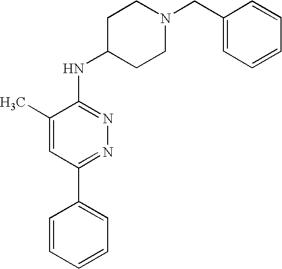 | MW01-1-02- L-F04 |
| 224 | 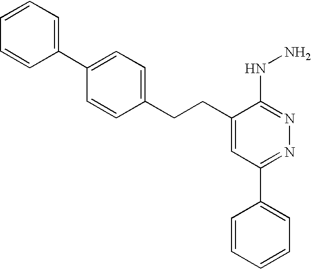 | MW01-1-02- L-F09 |
| 228 | 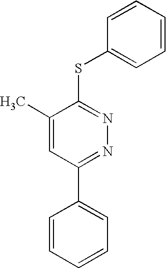 | MW01-1-03- L-A04 |
| 232 | 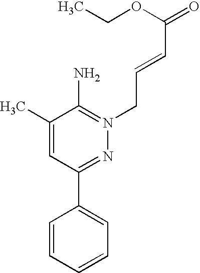 | MW01-1-03- L-C04 |
| 234 | 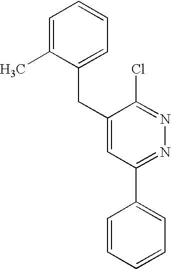 | MW01-1-03- L-E04 |
| 237 | 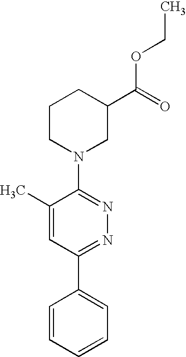 | MW01-1-03- L-E10 |
| 238 | 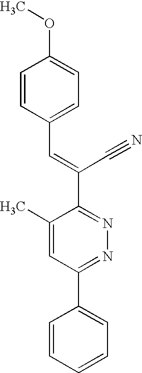 | MW01-1-03- L-G02 |
| 239 | 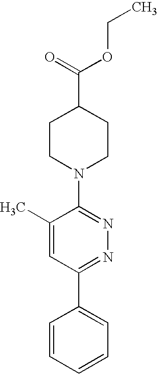 | MW01-1-03- L-H04 |
| 241 | 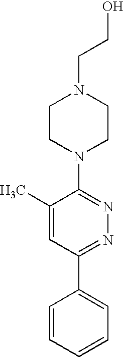 | MW01-1-04- L-D08 |
| 243 | 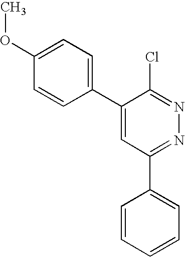 | MW01-1-04- L-E03 |
| 244 | 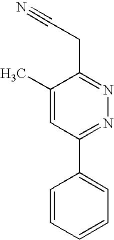 | MW01-1-04- L-E04 |
| 245 | 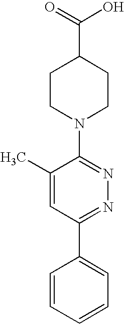 | MW01-1-04- L-E09 |
| 246 | 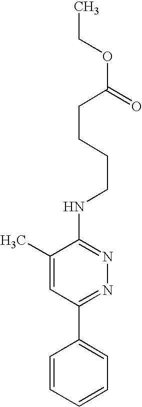 | MW01-1-04- L-F06 |
| 247 | 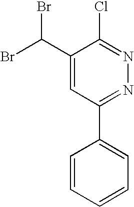 | MW01-1-04- L-G06 |
| 248 | 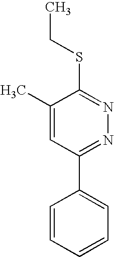 | MW01-1-04- L-H06 |
| 249 | 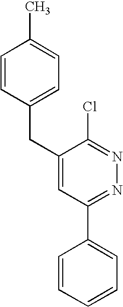 | MW01-1-04- L-H07 |
| 252 | 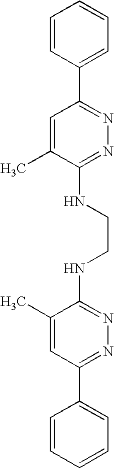 | MW01-1-05- L-F05 |
| 253 | 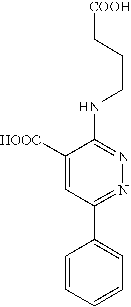 | MW01-1-05- L-G10 |
| 256 | 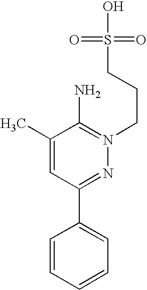 | MW01-1-05- L-H07 |
| 257 | 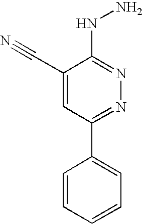 | MW01-1-05- L-H09 |
| 258 | 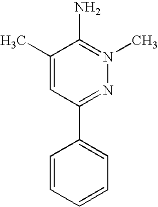 | MW01-1-05- L-H11 |
| 259 | 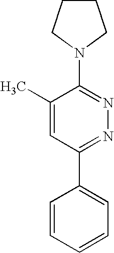 | MW01-1-07- L-E07 |
| 260 | 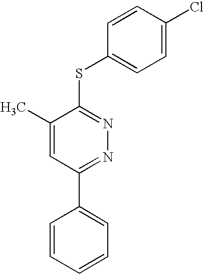 | MW01-1-07- L-G09 |
| 261 | 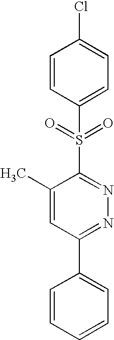 | MW01-1-07- L-H03 |
| 262 | 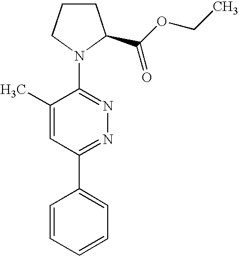 | MW01-1-07- L-H05 |
| 263 | 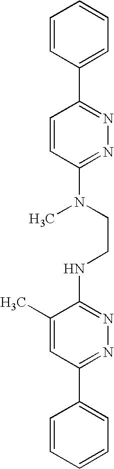 | MW01-1-07- L-H06 |
| 264 | 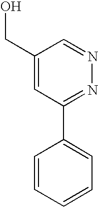 | MW01-1-08- L-C07 |
| 265 | 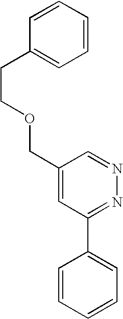 | MW01-1-08- L-C09 |
| 267 | 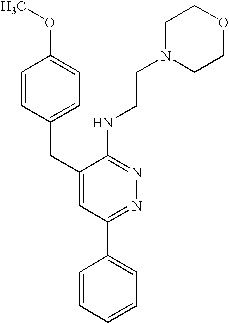 | MW01-1-08- L-E04 |
| 269 | 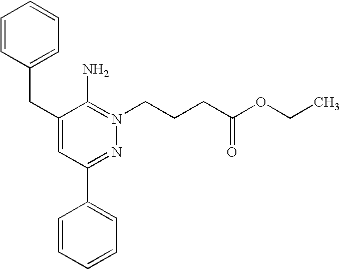 | MW01-1-09- L-G04 |
| 273 | 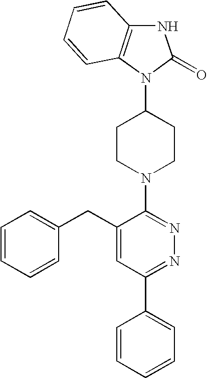 | MW01-1-09- L-G11 |
| 277 | 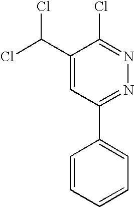 | MW01-1-15- L-B07 |
| 279 | 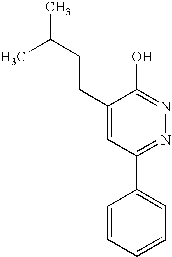 | MW01-1-15- L-B11 |
| 281 | 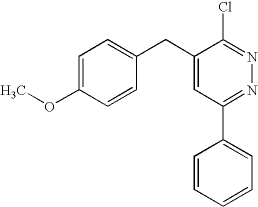 | MW01-1-15- L-D02 |
| 282 | 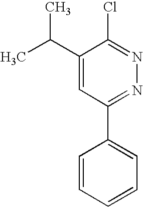 | MW01-1-15- L-D03 |
| 283 | 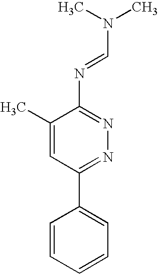 | MW01-1-15- L-E10 |
| 285 | 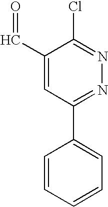 | MW01-1-15- L-H09 |
| 286 | 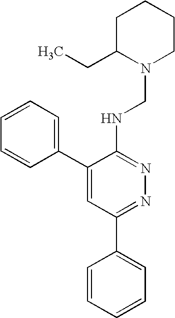 | MW01-1-16- L-E05 |
| 287 | 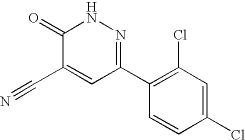 | MW01-1-01- L-F11 |
| 288 | 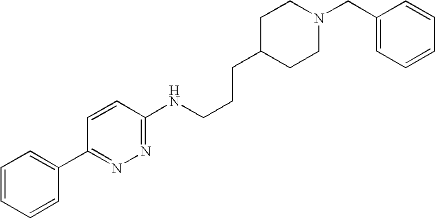 | MW01-1-17- L-B05 |
| 290 | 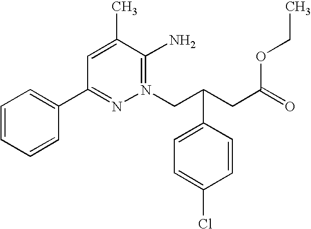 | MW01-1-16- L-E08 |
| 291 | 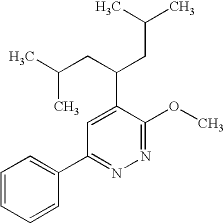 | MW01-1-16- L-G07 |
| 297 | 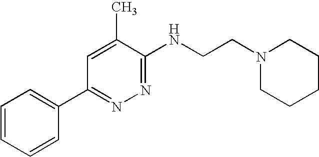 | MW01-1-17- L-F03 |
| 300 | 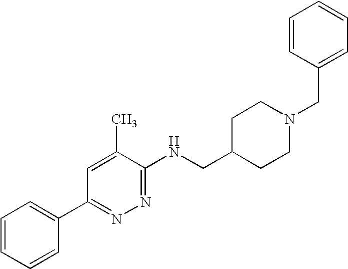 | MW01-1-18- L-B04 |
| 301 | 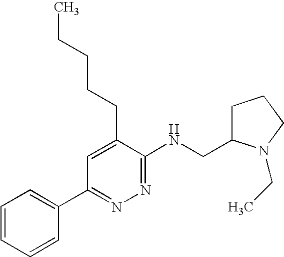 | MW01-1-18- L-B10 |
| 302 | 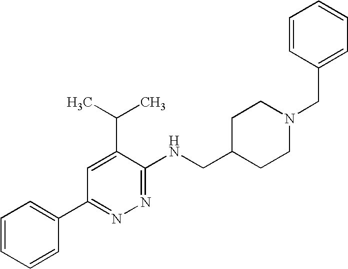 | MW01-1-18- L-B11 |
| 303 | 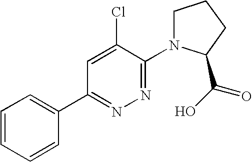 | MW01-1-18- L-C05 |
| 304 | 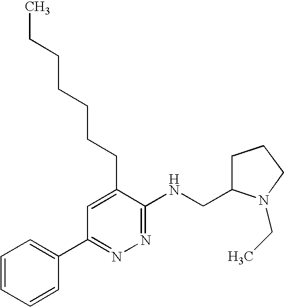 | MW01-1-18- L-C06 |
| 305 | 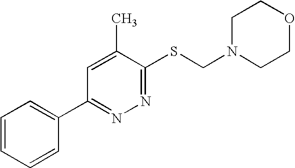 | MW01-1-18- L-C08 |
| 306 | 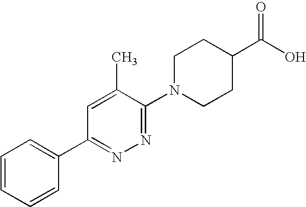 | MW01-1-18- L-C10 |
| 307 | 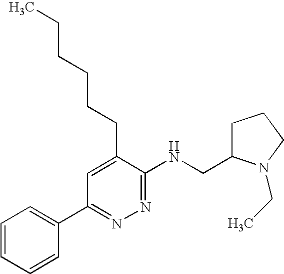 | MW01-1-18- L-D04 |
| 309 | 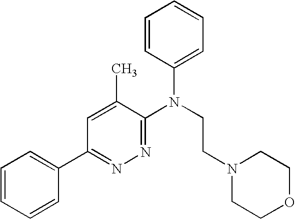 | MW01-2-03- L-C03 |
| 311 | 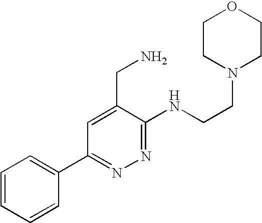 | MW01-2-03- L-D07 |
| 312 | 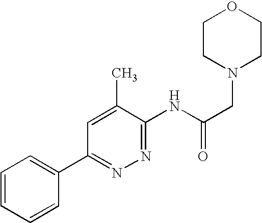 | MW01-2-03- L-D08 |
| 314 | 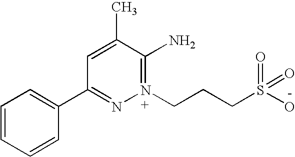 | MW01-2-03- L-G10 |
| 315 | 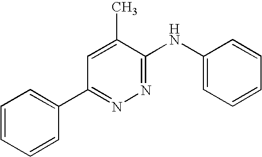 | MW01-2-06- L-F06 |
| 316 | 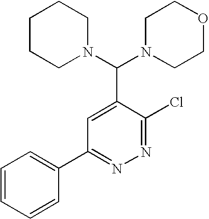 | MW01-2-09- L-B08 |
| 317 | 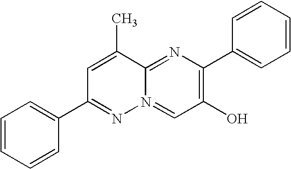 | MW01-2-09- L-E10 |
| 322 | 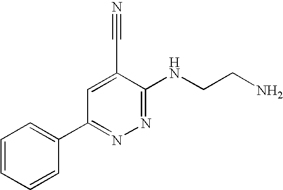 | MW01-2-20- L-B10 |
| 325 | 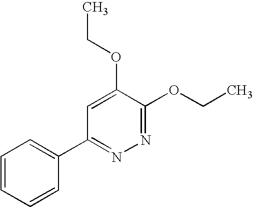 | MW01-2-24- L-A05 |
| 327 | 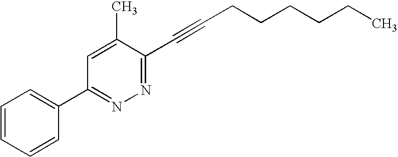 | MW01-3-01- L-G02 |
| 330 | 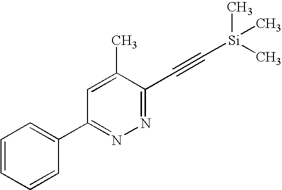 | MW01-3-01- L-G05 |
| 333 | 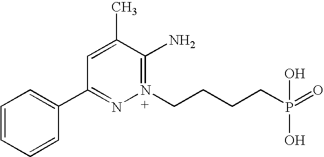 | MW01-3-06- L-B07 |
| 334 | 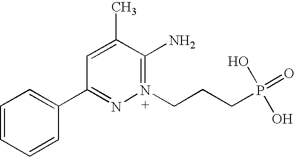 | MW01-3-06- L-B08 |
| 336 | 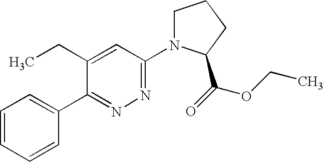 | MW01-1-07- L-G07 |
| 338 | 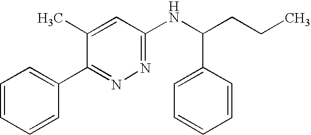 | MW01-1-08- L-D03 |
| 342 | 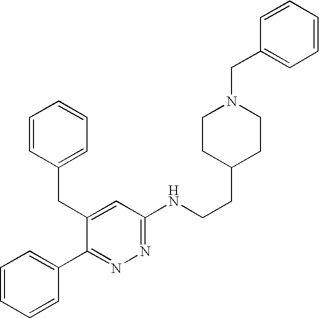 | MW01-1-16- L-E09 |
| 343 | 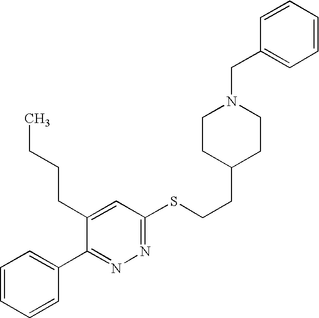 | MW01-1-17- L-C09 |
| 344 | 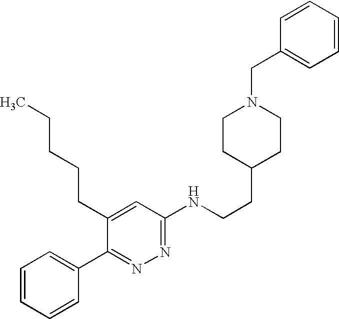 | MW01-1-17- L-E07 |
| 345 | 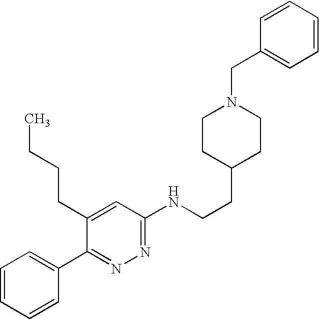 | MW01-1-17- L-E08 |
| 348 | 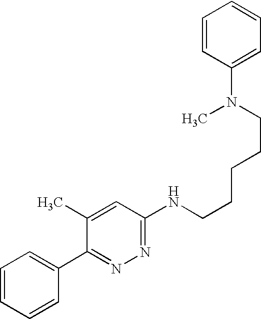 | MW01-1-18- L-A04 |
| 349 | 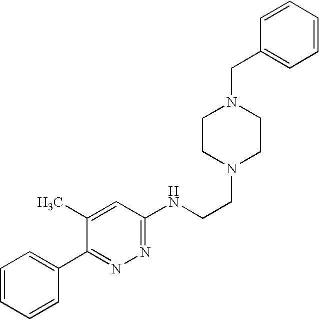 | MW01-1-18- L-B05 |
| 351 | 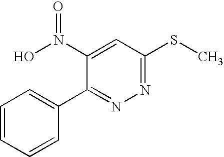 | MW01-2-33- L-A10 |
| 356 | 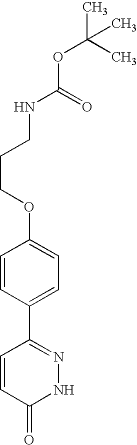 | MW01-1-01- L-E06 |
| 357 | 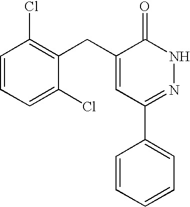 | MW01-1-01- L-H09 |
| 358 | 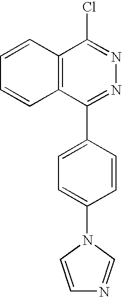 | MW01-1-05- L-D07 |
| 365 | 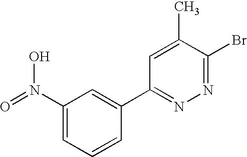 | MW01-1-03- L-D04 |
| 369 | 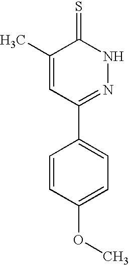 | MW01-1-04- L-G02 |
| 379 | 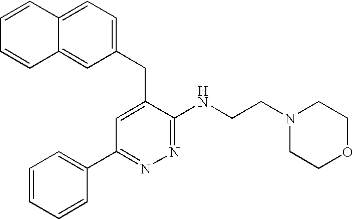 | MW01-2-24- L-E07 |
| | 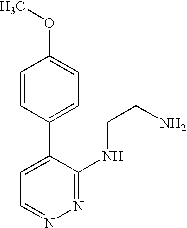 | MW01-01-01- L-B07 |
| | 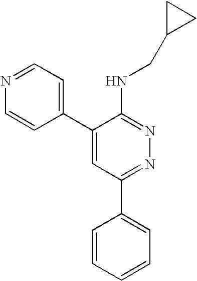 | MW01-7- 084WH |
| | 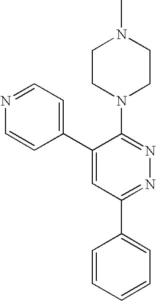 | MW01-7- 085WH |
| | 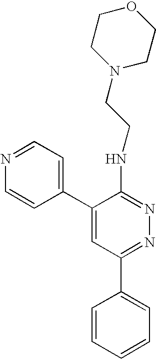 | MW01-7- 091WH |
| | 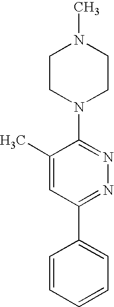 | MW01-10-12- L-G05 |
| | 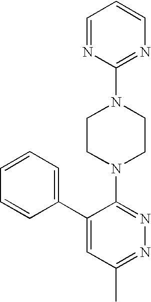 | MW01-7- 057WH |

TABLE 3
Compounds of the Formula II
2-(4-(6-phenylpyridazin-3-yl)piperazine-1-yl) pyrimidine and Derivatives

| No | Compound Table NU24020 Structure C1C | Final Code |
|---|---|---|
| | 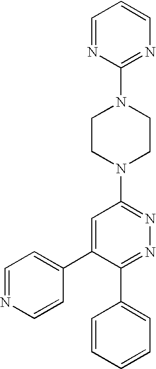 | MW01-2-069A- SRM |
| | 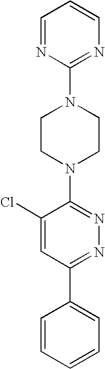 | MW01-6- 127WH |
| | 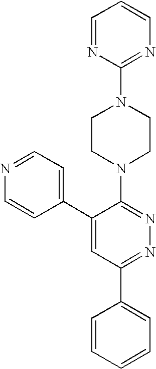 | MW01-6- 189WH |
| | 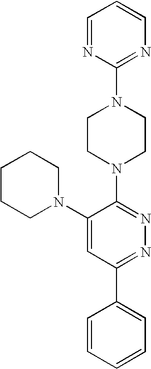 | MW01-7- 107WH |
| | 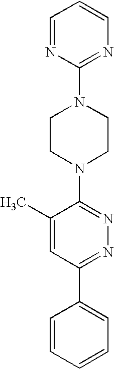 | WH 151SRM |
| | 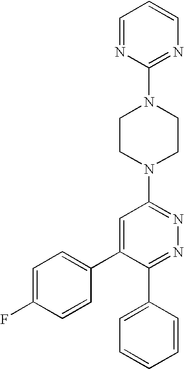 | MW01-2-069A- SRM |
| 29 | 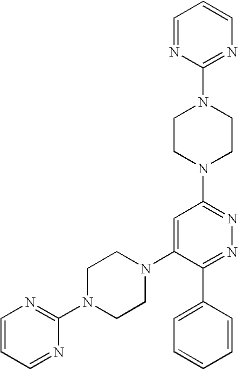 | MW01-1-030A- LKM |
| 33 | 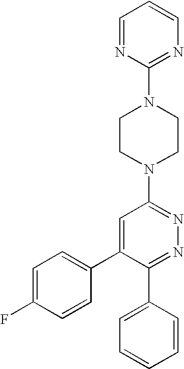 | MW01-2- 065LKM |
| 34 | 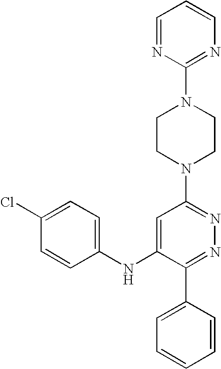 | MW01-2- 127LKM |
| 35 | 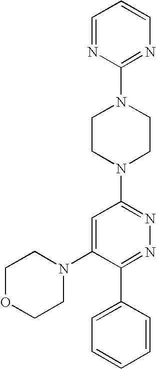 | MW01-2- 134LKM |
| 36 | 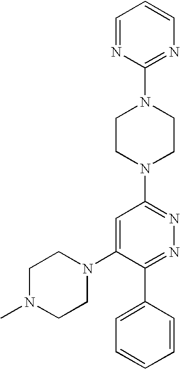 | MW01-2- 146LKM |
| 37 | 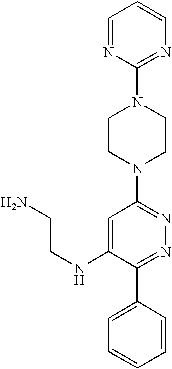 | MW01-2- 147LKM |
| 46 | 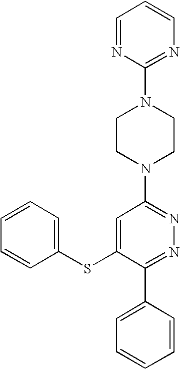 | MW01-1- 045MAS |
| 105 | 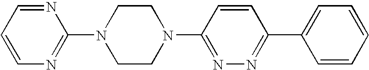 | MW01-9-038Z |
| 138 | 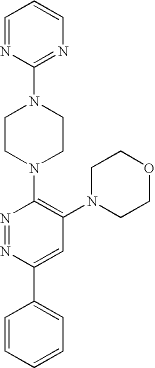 | MW01-2- 023SRM |
| 147 | 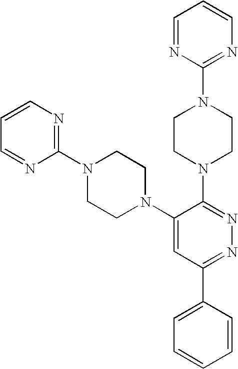 | MW01-2-177A- WH |
| 155 | 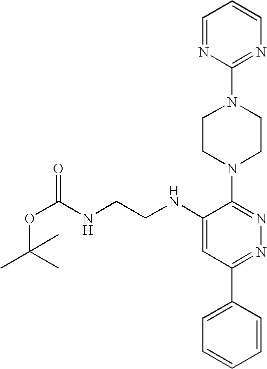 | MW01-2-191A- WH |
| 157 | 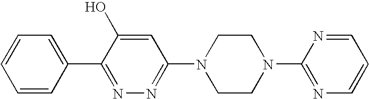 | MW01-3- 003WH |
| 160 | 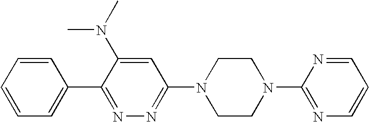 | MW01-3-019A- WH |
| 186 | 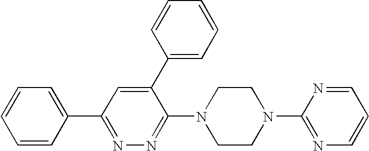 | MW01-5- 188WH |
| 252 | 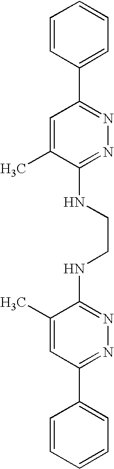 | MW01-1-05-L- F05 |
| 263 | 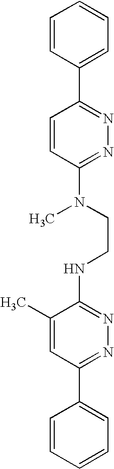 | MW01-1-07-L- H06 |

TABLE 4

| Compound | Code |
|---|---|
| 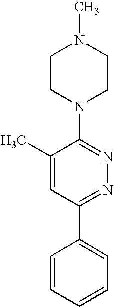 | MW01-01-02-L-G05 |
| 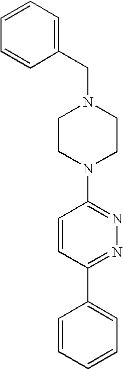 | MW01-01-03-L-E10 |
| 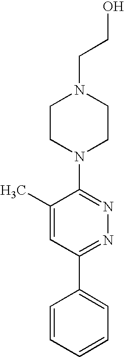 | MW01-01-04-L-D08 |
| 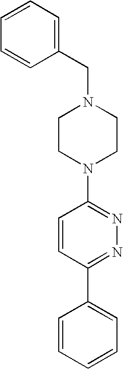 | MW01-01-18-L-A02 |
| 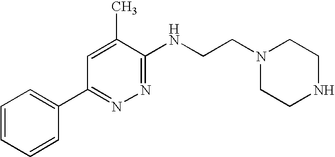 | MW01-01-18-L-C02 |
| 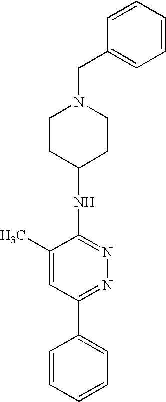 | MW01-02-03-L-G04 |
| 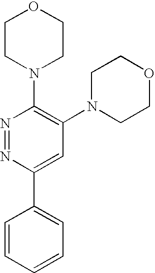 | MW01-2-18SRM |
| 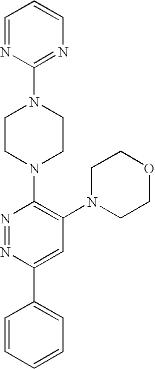 | MW01-2-023SRM |
| 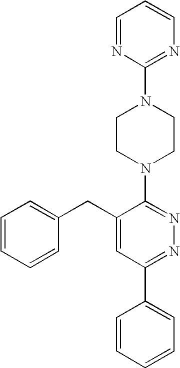 | MW01-2-141SRM |
| 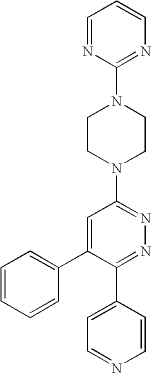 | MW01-2-163MAS |
| 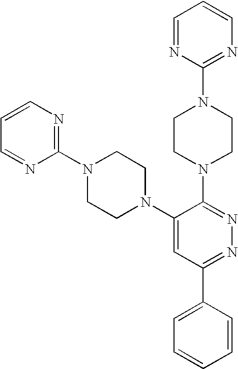 | MW01-2-177A-WH |
| 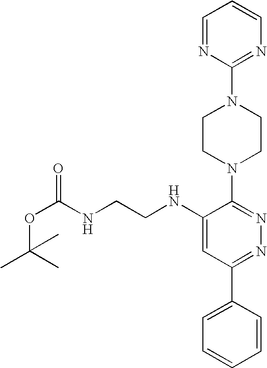 | MW01-2-191A-WH |
| 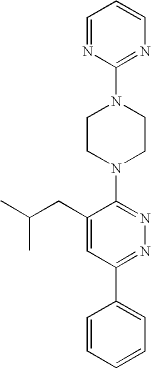 | MW01-3-024SRM |
| 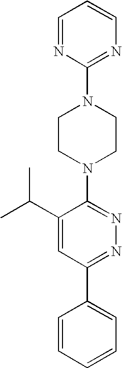 | MW01-3-027SRM |
| 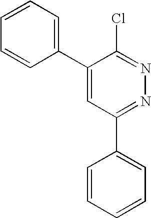 | MW01-3-057SRM |
| 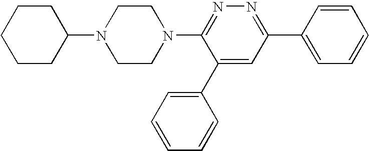 | MW01-3-065SRM |
| 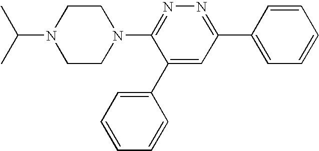 | MW01-3-066SRM |
| 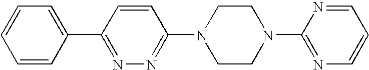 | MS01-3-183-WH |
| 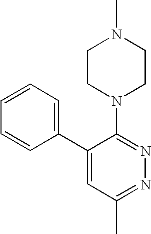 | MW01-4-179LKM |
| 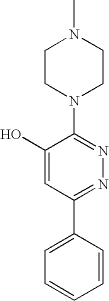 | MW01-4-188LKM |
| 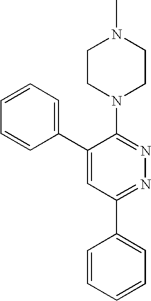 | MW01-7-027B-WH |
| 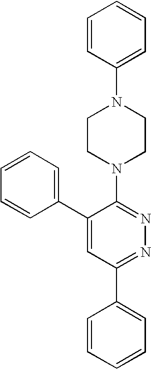 | MW01-7-029WH |
| 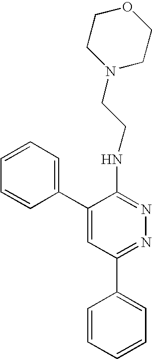 | MW01-7-031WH |
| 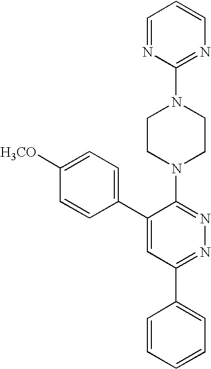 | MW01-7-100WH |
| 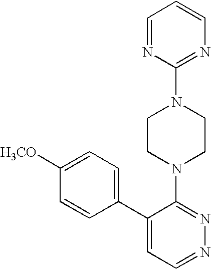 | MW01-7-102WH |
| 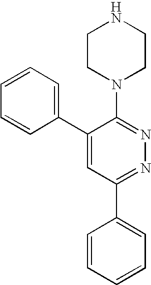 | MW01-7-133WH |
| 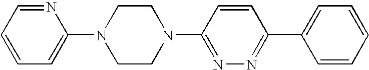 | MW01-9-039MZ |
| 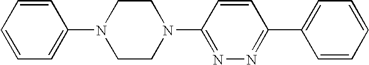 | MW01-9-040MZ |
| 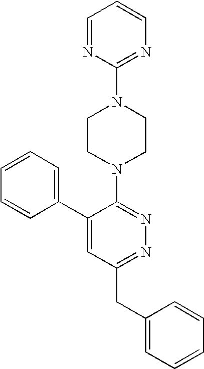 | MW01-210LPI |
| 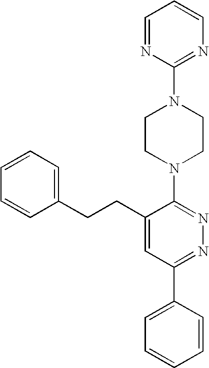 | MW01-2103LPI |

TABLE 5

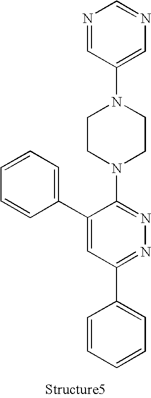

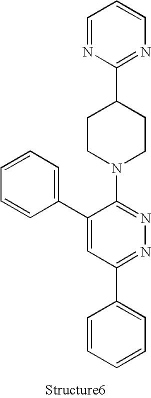

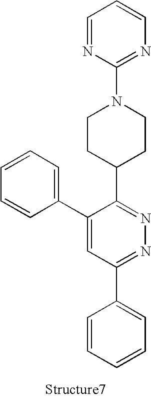

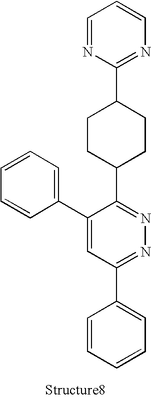

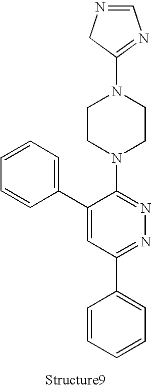

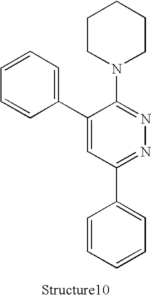

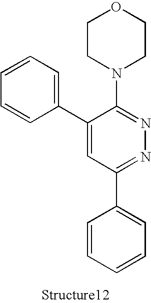

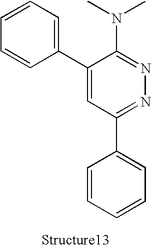

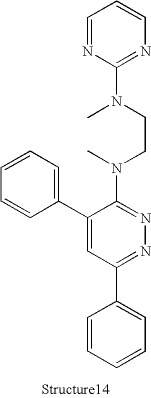

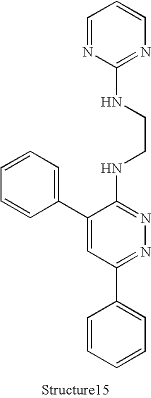

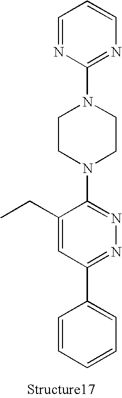

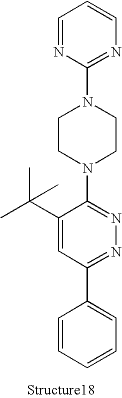

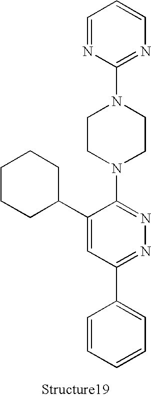

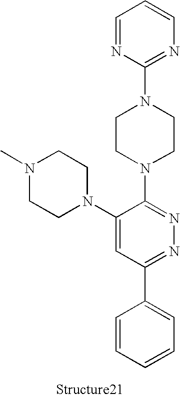

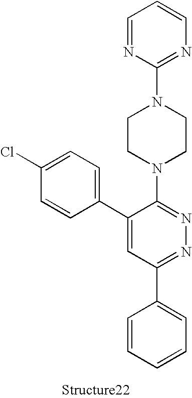

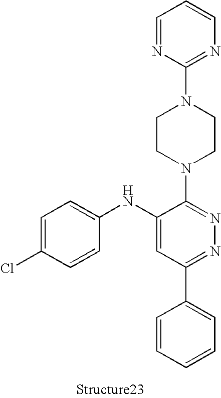

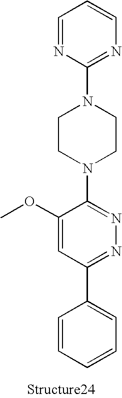

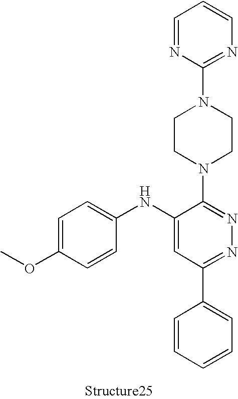

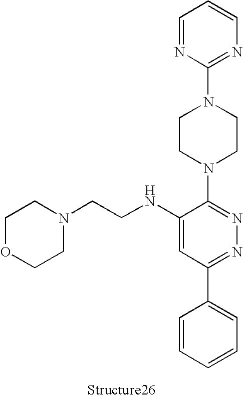

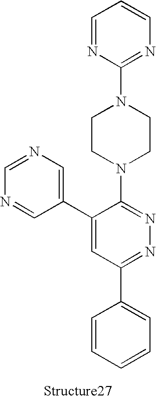

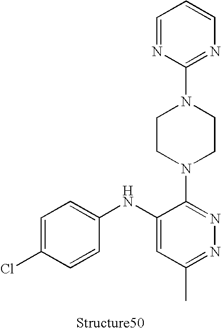

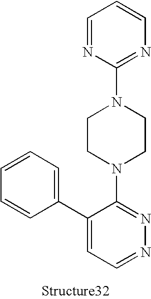

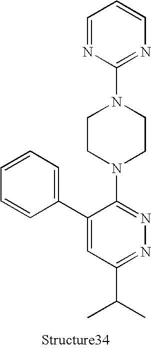

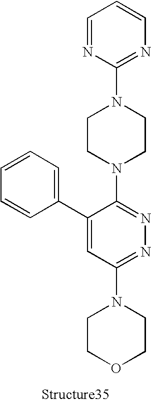

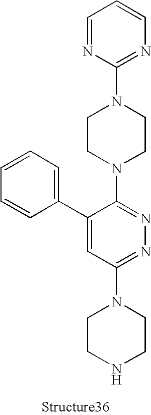

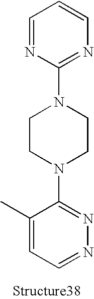

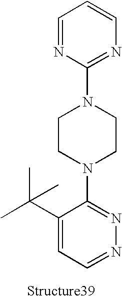

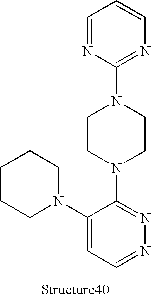

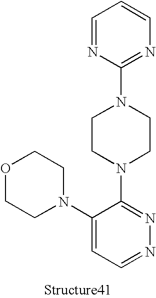

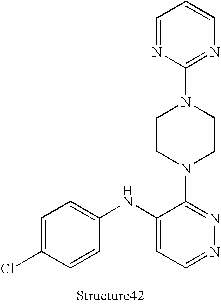

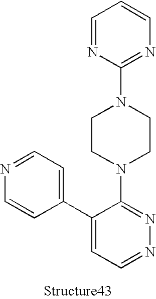

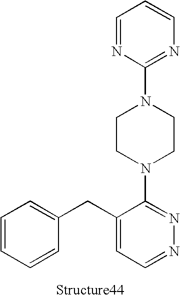

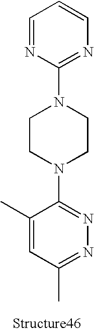

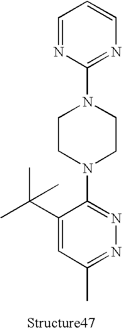

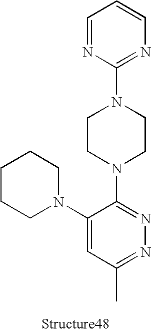

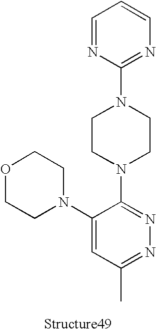

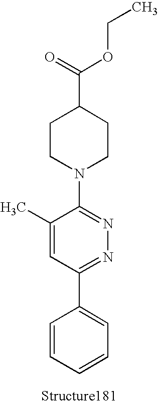

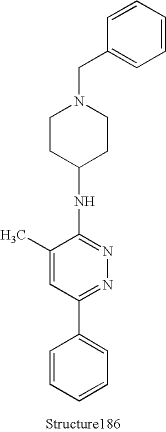

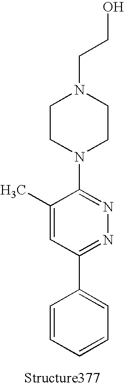

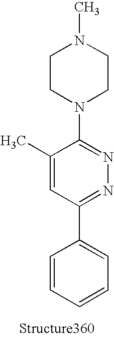

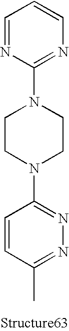

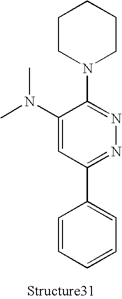

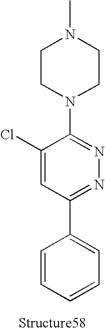

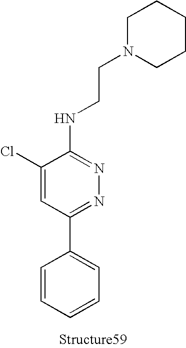

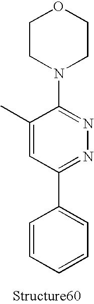

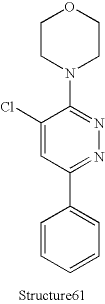

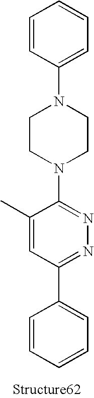

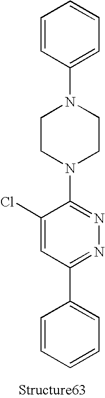

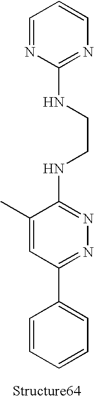

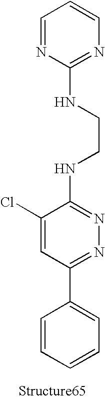

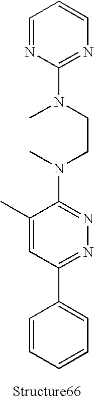

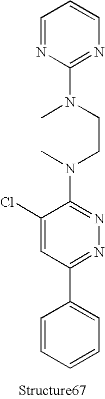

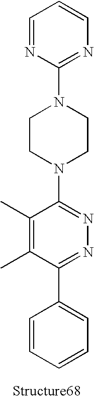

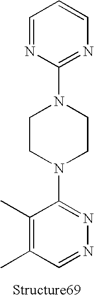

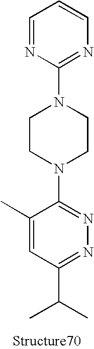

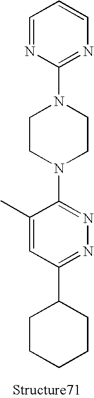

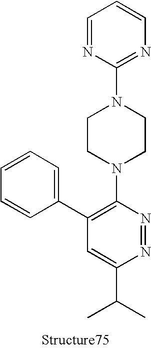

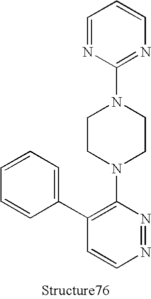

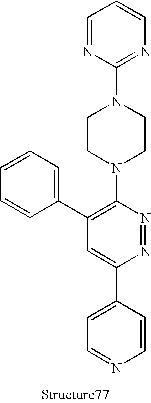

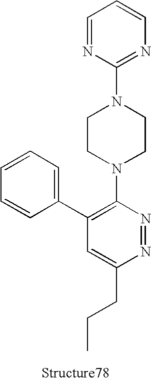

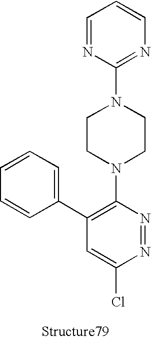

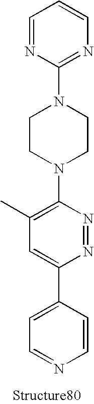

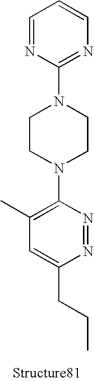

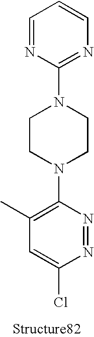

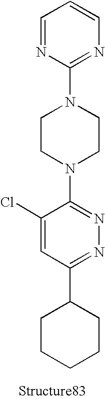

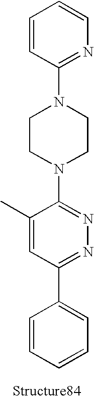

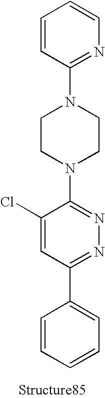

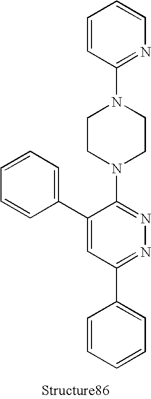

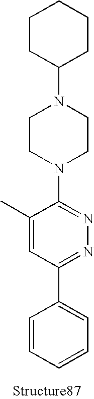

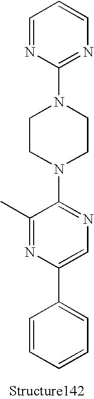

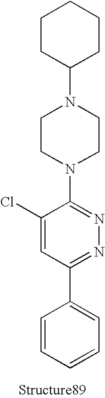

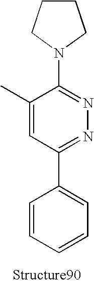

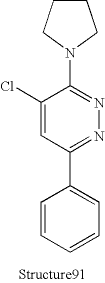

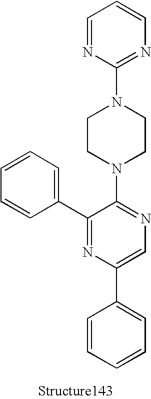

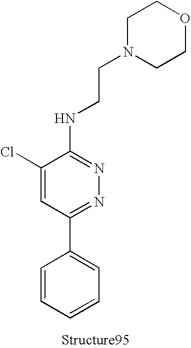

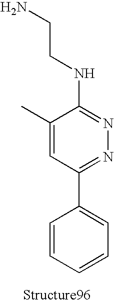

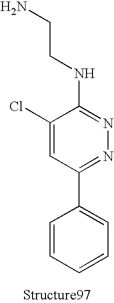

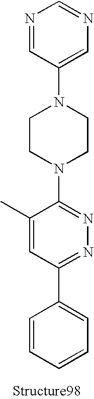

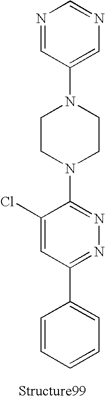

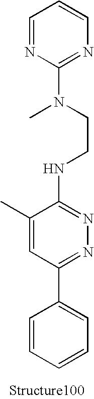

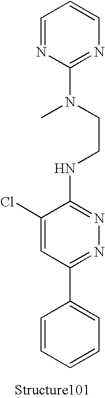

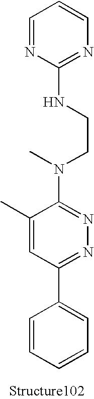

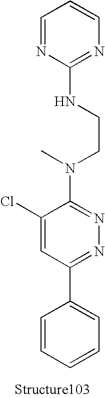

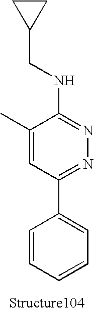

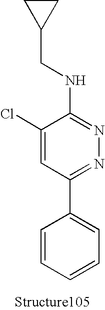

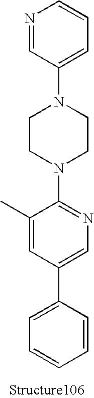

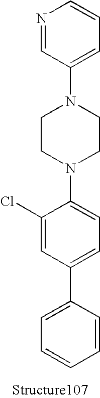

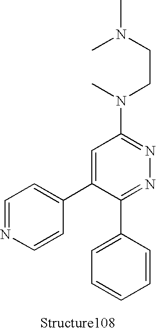

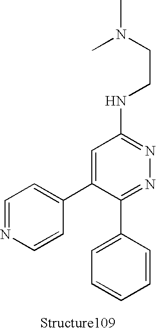

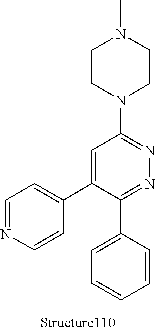

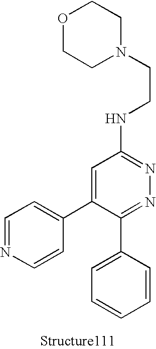

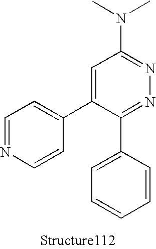

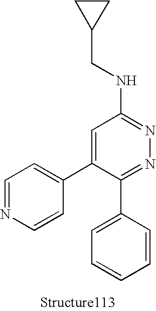

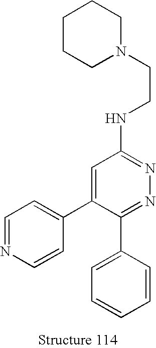

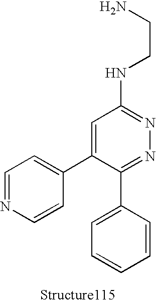

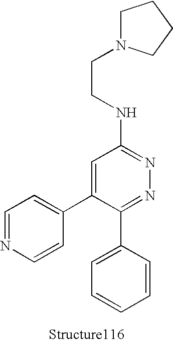

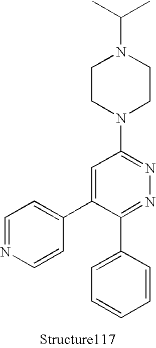

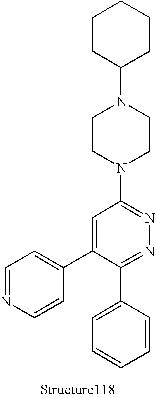

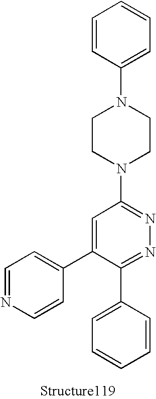

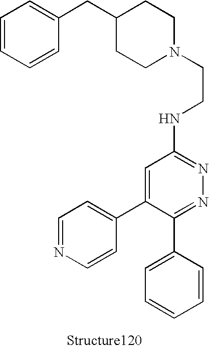

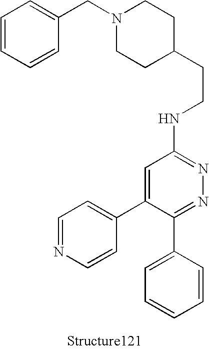

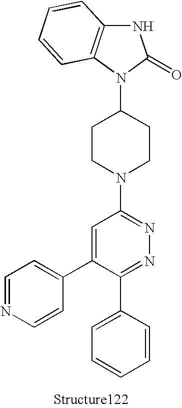

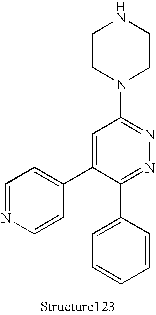

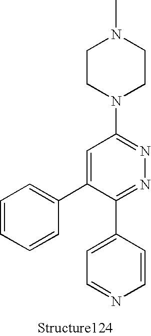

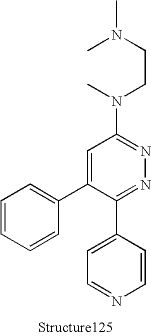

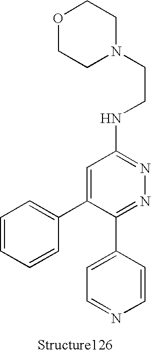

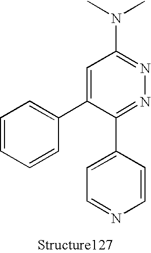

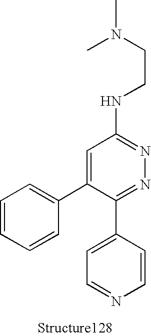

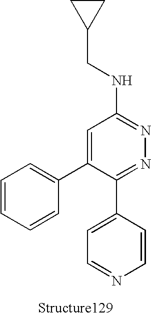

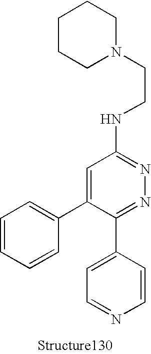

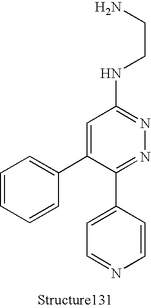

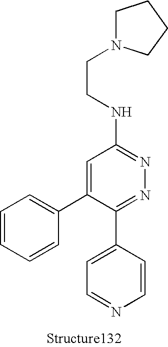

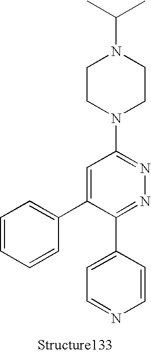

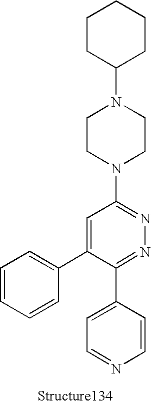

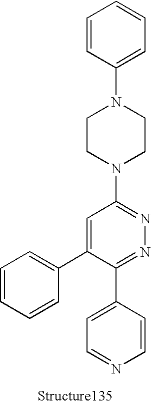

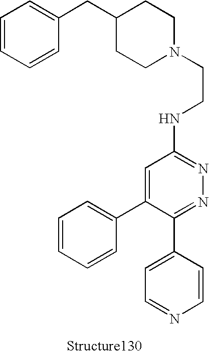

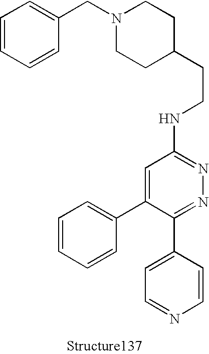

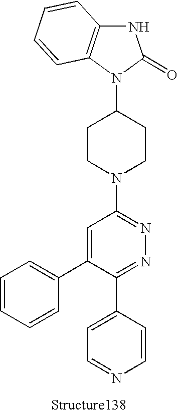

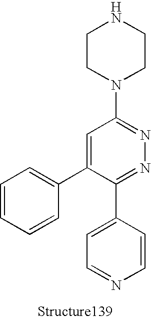

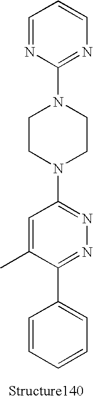

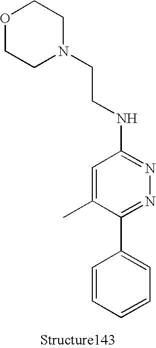

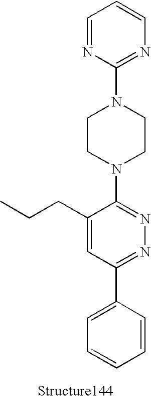

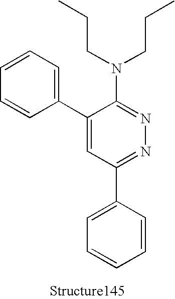

TABLE 6
Summary of experimental groups and outcome measures

| | | | | Endpoints | | |
|---|---|---|---|---|---|---|
| | Treatment | | Age at | ELISA or | | |
| Group (n) | P15 | P45 | sacrifice | Western | Immunohistochemistry | Behavior |
| P15 PBS (6) | PBS | | P16 | Cyt; FH; Cl | | |
| P15 KA (12) | KA | | P16 | Cyt; FH; Cl | | |
| P45 PBS (6) | | PBS | P46 | Cyt; FH; Cl | | |
| P45 KA (12) | | KA | P46 | Cyt; FH; Cl | | |
| PBS-PBS (20) | PBS | PBS | P55 | | FJB; GFAP; S100B; Iba1; | |
| | | | | | | Y maze |
| | | | | | FH; Cl; GLAST; GLT-I | |
| KA-PBS (19) | KA | PBS | P55 | | FJB; GFAP; S100B; Iba1; | Latency |
| | | | | | FH; Cl; GLAST; GLT-1 | Severity |
| | | | | | | Y maze |
| PBS-KA (20) | PBS | KA | P55 | | FJB; GFAP; S100B; Iba1; | Latency |
| | | | | | FH; Cl; GLAST; GLT-1 | Severity |
| KA-KA (12) | KA | KA | P55 | | FJB; GFAP; S100B; Iba1; | Latency |
| | | | | | FH; Cl; GLAST; GLT-1 | Severity |
| Total (107) | | | | | | |

P, postnatal day;
KA, Kainic acid;
Cyt, cytokines (IL-1β; 1L6; TNFα, S100B);
FJB, Fluoro-JadeB;
FH, Factor H;
Cl, Clusterin;
GLAST and GLT-1, Glutamate transporters;
Latency, time to seizure onset;
Severity, seizure severity following KA.

TABLE 7
Suppression of glial activation: summary of experimental groups,
and outcome measures

| | | | Endpoints | | |
|---|---|---|---|---|---|
| | Treatment | | | Immuno- | |
| Group (n) | (P15) | Recovery | ELISA | histochemistry | Behavior |
| PBS (12) | Sal | 12 hr | Cyt | | |
| KA (9) | Sal | 12 hr | Cyt | | |
| KA (13) | Mzc | 12 hr | Cyt | | |
| PBS (10) | Sal | 30 d | | GFAP; S100B | Y maze |
| KA (22) | Sal | 30 d | | GFAP; S100B | Y maze |
| KA (22) | Mzc | 30 d | | GFAP; S100B | Y maze |
| Total (88) | | | | | |

P, postnatal day;
KA, Kainic acid;
Mzc, Minozac;
Cyt, cytokines (IL-1β; IL6; TNFα, S100B)

### REFERENCES

The following are references used by the publication included in Example 1.
1. Giorgi F, Malhotra S, Hasson H, Veliskova J, Rosenbaum D, Moshé S. Effects of status epilepticus early in life on susceptibility to ischemic injury in adulthood. Epilepsia 2005; 46:490-498.
2. Koh S, Storey T, Santos T, Mian A, Cole A. Early-life seizures increase susceptibility to seizure-induced brain injury in adulthood. Neurology 1999; 53:915-921.
3. Dube C, Chen K, Eghbal Ahmadi M, Brunson K, Soltesz I, Baram T. Prolonged febrile seizures in the immature rat model enhance hippocampal excitability long term. Ann Neurol 2000; 34:774-780.
4. Schmid R, Tandon P, Stafstrom C, Holmes G. Effects of neonatal seizures on subsequent seizure-induced brain injury. Neurology 1999; 53:1754-1761.
5. French J, Williamson P, Thadani V, et al. Characteristics of medial temporal lobe epilepsy. I. Results of history and physical examination. Ann Neurol 1993; 34:774-780.
6. Haut S, Velisková J, Moshe S. Susceptibility of immature and adult brains to seizure effects. Lancet Neurol 2004; 3:608-617. 7.
7. Akiyama H, Barger S, Barnum S, et al. Inflammation and Alzheimer's Disease. Neurobiol Aging 2000; 21:383-421.
8. Mrak R, Griffin W. Glia and cytokines in progression of neurodegeneration. Neurobiol Aging 2005; 26:349-354.
9. Hagberg H, Mallard C. Effect of inflammation on central nervous system development and vulnerability. Curr Opin Neurol 2005; 18:117-123.
10. Wainwright M, Craft J, Griffin W, et al. Increased susceptibility of S100B transgenic mice to perinatal hypoxia-ischemia. Ann Neurol 2004; 56:61-67.
11. Van Eldik L J, Wainwright M S. The Janus face of glial-derived S100B: beneficial and detrimental functions in the brain. Restorative Neurol Neurosci 2003; 21:97-108.
12. Weiss S, Cataltepe O, Cole A. Anatomic studies of DNA fragmentation in rat brain after systemic kainic acid administration. Neuroscience 1996; 74:541-551.
13. Maragakis N, Rothstein J. Glutamate transporters: animal models to neurologic disease. Neurobiol Dis 2004; 15:461-473.
14. Huang Y, Bergles D. Glutamate transporters bring competition to the synapse. Curr Opin Neurobiol 2004; 14:346-352.
15. Zhang G, Raol Y H, Hsu F, Brooks-Kayal A. Long-term alterations in glutamate receptor and transporter expression following early-life seizures are associated with increased seizure susceptibility. J Neurochem 2004; 88:91-101.
16. Strohmeyer R, Ramirez M, Cole G, Mueller K, Rogers J. Association of factor H of the alternative pathway of complement with agrin and complement receptor 3 in the Alzheimer's disease brain. J Neuroimmunol 2002; 131:135-146.
17. Han B, DeMattos R, Dugan L, et al. Clusterin contributes to caspase-3-independent brain injury following neonatal hypoxia-ischemia. Nature Med 2001; 7:338-343.
18. Dragunow M, Preston K, Dodd J, Young D, Lawlor P, Christie D. Clusterin accumulates in dying neurons following status epilepticus. Mol Brain Res 1995; 32:279-290.
19. Hu W, Ralay Ranaivo H, Roy S, et al. Development of a novel therapeutic suppressor of brain pro-inflammatory cytokine up-regulation that attenuates synaptic dysfunction and behavioral deficits. Bioorgan Med Chem Lett 2007; 17:414-418.
20. Perry V, Cunningham C, Holmes C. Systemic infections and inflammation affect chronic neurodegeneration. Nat Rev Immunol 2007; doi: 10.1038/nri2015.

21. Wing L, Behanna H, Van Eldik L, Watterson D, Ralay Ranaivo H. De novo and molecular target-independent discovery of orally bioavailable lead compounds for neurological disorders. Curr Alzheimer Res 2006; 3:205-214.
22. Rizzi M, Perego C, Aliprandi M, et al. Glia activation and cytokine increase in rat hippocampus by kainic acid-induced status epilepticus during postnatal development. Neurobiol Dis 2003; 14.
23. Paxinos G, Watson C. The Rat Brain in Stereotaxic Coordinates. 2nd edition. Sydney: Academic Press, 1986.
24. Schmued L, Hopkins K. Fluoro-Jade B: a high affinity fluorescent marker for the localization of neuronal degeneration. Brain Res 2000; 874:123-130.
25. Wainwright M, Kohli R, Whitington P, Chace D. Carnitine treatment inhibits increases in cerebral carnitine esters and glutamate detected by mass spectrometry following hypoxiaischemia in newborn rats. Stroke 2006; 37:524-530.
26. Ralay Ranaivo H, Craft J, Hu W, et al. Glia as a therapeutic target: selective suppression of human amyloid-B-induced up-regulation of brain proinflammatory cytokine production attenuates neurodegeneration. J Neurosci 2006; 26:662-670.
27. Weiss C, Shroff A, Disterhoft J. Spatial learning and memory in aging C57BL/6 mice. Neurosci Res Commun 1998; 23:77-92.
28. Strohmeyer R, Rogers J. Molecular and cellular mediators of Alzheimer's disease inflammation. J Alz Dis 2001; 3:131-157.
29. Holmes G. Effects of seizures on brain development: lessons from the laboratory. Pediatr Neurol 2005; 33:1-11.
30. Holmes G, Ben An Y. Seizures in the developing brain: perhaps not so benign after all. Neuron 1998; 21:1231-1234.
31. Sayin U, Sutula T, Stafstrom C. Seizures in the developing brain cause adverse long-term effects on spatial learning and anxiety. Epilepsia 2004; 45:1539-1548.
32. Letty S, Lerner-Natoli M, Rondouin G. Differential impairments of spatial memory and social behavior in two models of limbic epilepsy. Epilepsia 1995; 36:973-982.
33. Jensen F, Blume H, Alvarado I, Firkusny I, Geary C. NBQX blocks acute and late epileptogenic effects of perinatal hypoxia. Epilepsia 1995; 36:966-972.
34. Sanchez R, Koh S, Rio C, et al. Decreased glutamate receptor 2 expression and enhanced epileptogenesis in immature rat hippocampus after perinatal hypoxia-induced seizures. J Neurosci 2001; 21:8154-8163.
35. Cardona A, Pioro E, Sasse M, et al. Control of microglial neurotoxicity by the fractalkine receptor. Nature Neurosci 2006; 9:917-924.
36. Minghetti L. Role of inflammation in neurodegenerative diseases. Curr Opin Neurol 2005; 18:315-321.
37. Vezzani A, Granata T. Brain inflammation in epilepsy: Experimental and clinical evidence. Epilepsia 2005; 46:1724-1743.
38. Craft J, Watterson D, Van Eldik L. Neuroinflammation: a potential therapeutic target in Alzheimer's disease and related disorders. Exp Opin Therap Targets 2005; 9:887-900.
39. Sheng J, Kazuhiro I, Skinner R, et al. In vivo and in vitro evidence supporting a role for the inflammatory cytokine interleukin-1 as a driving force in Alzheimer pathogenesis. Neurobiol Aging 1996; 17:761-766.
40. Ravizza T, Rizzi M, Perego C, et al. Inflammatory response and glia activation in developing rat hippocampus after status epilepticus. Epilepsia 2005; 46:S113-S117.
41. Vezzani A, Richichi C, Aliprandi M, et al. Functional role of inflammatory cytokines and antiinflammatory molecules in seizures and epileptogenesis. Epilepsia 2002; 43:S30-S35.
42. Rothermundt M, Peters M, Prehn J H, Arolt V. S100B in brain damage and neurodegeneration. Microscopy Research & Technique 2003; 60:614-632.
43. Leviton A, Darnmann O. Brain damage markers in children. Neurobiological and clinical aspects. Acta Paediatrica 2002; 91:9-13.
44. Craft J, Watterson D, Marks A, Van Eldik L. Enhanced susceptibility of S-100B transgenic mice to neuroinflammation and neuronal dysfunction induced by intracerebroventricular infusion of human β-amyloid. GLIA 2005; 51:209-216.
45. Verbitsky M, Yonan A, Malleret G, Kandel E, Giliam T, Pavlidis P. Altered hippocampal transcript profile accompanies an age-related spatial memory deficit in mice. Learining and Memory 2004; 11:253-260.
46. Rao V, Dogan A, Bowen K, Todd K, Dempsey R. Antisense knockdown of the glial glutamate transporter GLT-1 exacerbates hippocampal damage following traumatic injury to rat brain. Eur J Neurosci 2001; 13:119-128.

1. A pharmaceutical composition for treating a disorder characterized by conduction disturbances, electroconvulsions and/or seizures comprising a pyridazine compound in a therapeutically effective amount for treating the disorder, and a pharmaceutically acceptable carrier, excipient, or vehicle.

2. A pharmaceutical composition according to claim 1 wherein the pyridazine compound comprises a pyridazinyl radical pendant with an aryl or substituted aryl, a heteroaryl or substituted heteroaryl.

3. A pharmaceutical composition according to claim 2 wherein the heteroaryl is piperazinyl substituted with pyrimidinyl or pyridinyl.

4. A pharmaceutical composition according to claim 1 wherein the pyridazine compound has the Formula Ia or Ib 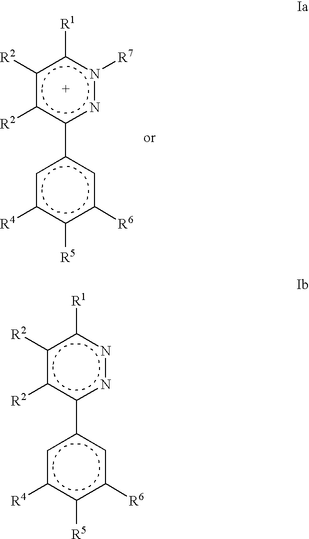
wherein R¹, R², and R³ are independently hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide; R⁷ is substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide or R⁷ may be absent and there is a double bond between N at position 1 and C at position 6; R⁴, R⁵, and R⁶ are independently hydrogen, alkyl, alkoxy, halo, or nitro; or R¹ and R², R¹ and R⁷, or R² and R³ may form a heteroaryl or heterocyclic ring; or an isomer or a pharmaceutically acceptable salt thereof.

5. A pharmaceutical composition according to claim 1 wherein the pyridazine compound has the Formula II: 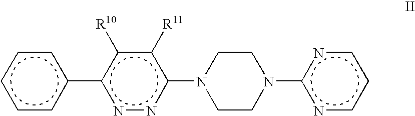
wherein R¹⁰ and R¹¹ are independently hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, phosphonate, cyano, halo, silyl, silyloxy, silylalkyl, silylthio, ═O, ═S, carboxyl, carbonyl, carbamoyl, or carboxamide; or an isomer or a pharmaceutically acceptable salt thereof.

6. A pharmaceutical composition according to claim 1 wherein the pyridazine compound has the Formula III: 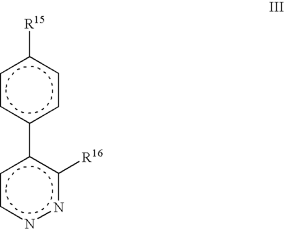
wherein R¹⁵ and R¹⁶ are independently substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide; or an isomer or a pharmaceutically acceptable salt thereof.

7. A pharmaceutical composition according to claim 1 wherein the pyridazine compound has the Formula IV: 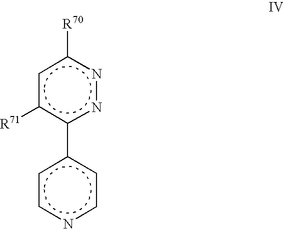
wherein R⁷⁰ is substituted or unsubstituted hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate, amino, imino, azido, thiol, thioalkyl, thioalkoxy, thioaryl, nitro, ureido, cyano, halo, silyl, silyloxy, silylalkyl, silylthio, ═O, ═S, carboxyl, phosphonate, carbonyl, carbamoyl, or carboxamide, especially heterocyclic, heteroaryl, amino, and substituted amino and R⁷¹ is aryl or substituted aryl; or an isomer or a pharmaceutically acceptable salt thereof.

8. A pharmaceutical composition according to claim 1 wherein the pyridazine compound has the Formula V: 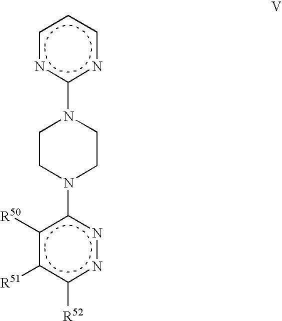
wherein R⁵⁰, R⁵¹, and R⁵² are independently hydrogen, hydroxyl, alkyl, alkenyl, alkynyl, alkylene, alkenylene, alkoxy, alkenyloxy, cycloalkyl, cycloalkenyl, cycloalkynyl, cycloalkoxy, aryl, aryloxy, arylalkoxy, aroyl, heteroaryl, heterocyclic, acyl, acyloxy, sulfonyl, sulfinyl, sulfenyl, sulfoxide, sulfate, sulfonate amino, imino, azido, thiol, thioalkyl, thioalkoxy, thioaryl, nitro, ureido, cyano, halo, silyl, silyloxy, silylalkyl, silylthio, ═O, ═S, phosphonate, carboxyl, carbonyl, carbamoyl, or carboxamide; or an isomer or a pharmaceutically acceptable salt thereof.

9. A pharmaceutical composition according to claim 1 wherein the pyridazine compound does not include the compounds depicted in Table 1.

10. A pharmaceutical composition according to claim 1 wherein the pyridazine compound is 2-(4-(4-methyl-6-phenylpyridazin-3-yl)piperazin-1-yl)pyrimidine dihydrochloride salt.

11. A method for treating and/or preventing a disorder characterized by conduction disturbances, electroconvulsions and/or seizures in a subject comprising administering a therapeutically effective amount of a composition according to claim 1.

12. A method according to claim 11 wherein the disorder is epilepsy.

13. A method according to claim 11 wherein subject is a pediatric patient.

14. A method of using a composition according to claim 1 comprising treating and/or preventing a disorder characterized by conduction disturbances, electroconvulsions and/or seizures.

15. A kit comprising a pyridazine compound or a composition as claimed in claim 1 for preventing and/or treating a disorder characterized by conduction disturbances, electroconvulsions and/or seizures, a container, and instructions for its use.
